SEQUENCE LISTING (126 sequences)

SEQ ID NO: 1 - protein, 10 aa, Artificial
  CDR sequence of BCMA VHH
GRLFRYTDMA

SEQ ID NO: 2 - protein, 16 aa, Artificial
  CDR sequence of BCMA VHH
FSYVSGRTNYADSVKG

SEQ ID NO: 3 - protein, 18 aa, Artificial
  CDR sequence of BCMA VHH
AQGRSPYRWGSYSSMFGS

SEQ ID NO: 4 - protein, 10 aa, Artificial
  CDR sequence of BCMA VHH
GRTFSSAFMG

SEQ ID NO: 5 - protein, 17 aa, Artificial
  CDR sequence of BCMA VHH
GIRWGAGNTYYADSVKG

SEQ ID NO: 6 - protein, 12 aa, Artificial
  CDR sequence of BCMA VHH
ARWRWGPKYYDY

SEQ ID NO: 7 - protein, 10 aa, Artificial
  CDR sequence of BCMA VHH
GRTFGWYTMG

SEQ ID NO: 8 - protein, 17 aa, Artificial
  CDR sequence of BCMA VHH
AISPSGGATYYADSVKG

SEQ ID NO: 9 - protein, 12 aa, Artificial
  CDR sequence of BCMA VHH
NARLWGLGIRNY

SEQ ID NO: 10 - protein, 10 aa, Artificial
  CDR sequence of BCMA VHH
GRSFSRYAMG

SEQ ID NO: 11 - protein, 16 aa, Artificial
  CDR sequence of BCMA VHH
SITRFGSTNYADSMKG

SEQ ID NO: 12 - protein, 20 aa, Artificial
  CDR sequence of BCMA VHH
AKERWYGSRWHVANYLQYDS

SEQ ID NO: 13 - protein, 9 aa, Artificial
  CDR sequence of CD38 VHH
GTVGPTVMG

SEQ ID NO: 14 - protein, 17 aa, Artificial
  CDR sequence of CD38 VHH
LIGWTGGVTAYADSVKD

SEQ ID NO: 15 - protein, 17 aa, Artificial
  CDR sequence of CD38 VHH
AADSNRRRYYGPYEYDS

SEQ ID NO: 16 - protein, 13 aa, Artificial
  CDR sequence of CD3 VHH
KASGYTFTSYTMH

SEQ ID NO: 17 - protein, 17 aa, Artificial
  CDR sequence of CD3 VHH
YINPSSGYTKYNQKFKD

SEQ ID NO: 18 - protein, 12 aa, Artificial
  CDR sequence of CD3 VHH
ARWQDYDVYFDY

SEQ ID NO: 19 - protein, 13 aa, Artificial
  CDR sequence of CD3 VHH
KASGYTFTRYTMH

SEQ ID NO: 20 - protein, 17 aa, Artificial
  CDR sequence of CD3 VHH
YINPSRGYTNYNQKFKD

SEQ ID NO: 21 - protein, 12 aa, Artificial
  CDR sequence of CD3 VHH
ARYYDDHYCLDY

SEQ ID NO: 22 - protein, 13 aa, Artificial
  CDR sequence of CD3 VHH
KASGYTFTRYTMH

SEQ ID NO: 23 - protein, 17 aa, Artificial
  CDR sequence of CD3 VHH
YINPSRGYTNYNQKFKD

SEQ ID NO: 24 - protein, 12 aa, Artificial
  CDR sequence of CD3 VHH
ARYYDDHYCLDY

SEQ ID NO: 25 - protein, 10 aa, Artificial
  CDR sequence of CD3 VH
GYTFTSYTMH

SEQ ID NO: 26 - protein, 17 aa, Artificial
  CDR sequence of CD3 VH
YINPSSGYTKYNQKFKD

SEQ ID NO: 27 - protein, 12 aa, Artificial
  CDR sequence of CD3 VH
ARWQDYDVYFDY

SEQ ID NO: 28 - protein, 10 aa, Artificial
  CDR sequence of CD3 VL
RASSSVSYMH

SEQ ID NO: 29 - protein, 7 aa, Artificial
  CDR sequence of CD3 VL
DTSNLAS

SEQ ID NO: 30 - protein, 9 aa, Artificial
  CDR sequence of CD3 VL
QQWSSNPPT

SEQ ID NO: 31 - protein, 10 aa, Artificial
  CDR sequence of CD3 VL
RASSSVSYMH

SEQ ID NO: 32 - protein, 7 aa, Artificial
  CDR sequence of CD3 VL
AASNRAS

SEQ ID NO: 33 - protein, 9 aa, Artificial
  CDR sequence of CD3 VL
QQWSSNPPT

SEQ ID NO: 34 - protein, 10 aa, Artificial
  CDR sequence of CD3 VL
RASSSVSYMH

SEQ ID NO: 35 - protein, 7 aa, Artificial
  CDR sequence of CD3 VL
ATSNRAS

SEQ ID NO: 36 - protein, 9 aa, Artificial
  CDR sequence of CD3 VL
QQWSSNPPT

SEQ ID NO: 37 - protein, 10 aa, Artificial
  CDR sequence of CD3 VL
RASQSVSYMH

SEQ ID NO: 38 - protein, 7 aa, Artificial
  CDR sequence of CD3 VL
ATSNRAS

SEQ ID NO: 39 - protein, 9 aa, Artificial
  CDR sequence of CD3 VL
QQRSSNPPT

SEQ ID NO: 40 - protein, 10 aa, Artificial
  CDR sequence of CD3 VL
RASSSVSYMH

SEQ ID NO: 41 - protein, 7 aa, Artificial
  CDR sequence of CD3 VL
DTSNLAS

SEQ ID NO: 42 - protein, 9 aa, Artificial
  CDR sequence of CD3 VL
QQRSSNPLT

SEQ ID NO: 43 - protein, 10 aa, Artificial
  CDR sequence of CD3 VL
RASQSVSYMH

SEQ ID NO: 44 - protein, 7 aa, Artificial
  CDR sequence of CD3 VL
DASNRAS

SEQ ID NO: 45 - protein, 9 aa, Artificial
  CDR sequence of CD3 VL
QQWSSWPPT

SEQ ID NO: 46 - protein, 124 aa, Artificial
  BCMA VHH sequence
QVQLVESGGGLVQAGGSLRLSCASSGRLFRYTDMAWYRQAPGKQRELVAFSYVSGRTNYADSVKGRFTISRDNVKKVVYLQMSSLTPEDTAVYYCAQGRSPYRWGSYSSMFGSWGQGTLVTVSS

SEQ ID NO: 47 - protein, 119 aa, Artificial
  BCMA VHH sequence
QVQLVESGGGLVQAGGSLRLSCAASGRTFSSAFMGWFRQAPGKERDFVAGIRWGAGNTYYADSVKGRFTISRDNAKNMVYLQMNSLEPEDTAVYYCARWRWGPKYYDYWGQGTLVTVSS

SEQ ID NO: 48 - protein, 119 aa, Artificial
  BCMA VHH sequence
QVQLVESGGGLVQAGGSLRLSCTASGRTFGWYTMGWFRQGPGKEREFVAAISPSGGATYYADSVKGRFTISRDYAKNAVYLQMNSLKPEDTAVYYCNARLWGLGIRNYWGQGTLVTVSS

SEQ ID NO: 49 - protein, 126 aa, Artificial
  BCMA VHH sequence
QVQLVESGGGLVQVGGSLRLSCAASGRSFSRYAMGWFRQAPGKERELVASITRFGSTNYADSMKGRFTVSRDNSKNTLYLHMNNLKPDDTAVYYCAKERWYGSRWHVANYLQYDSWGQGTLVTVSS

SEQ ID NO: 50 - protein, 123 aa, Artificial
  CD38 VHH sequence
EVQLVESGGGLVQAGGSLRLSCAVSGTVGPTVMGWFRQAPGKKREFVALIGWTGGVTAYADSVKDRFTISRDCAENRVDLQMDSLKPEDTAVYYCAADSNRRRYYGPYEYDSWGQGTLVTVSS

SEQ ID NO: 51 - protein, 119 aa, Artificial
  CD3 VHH sequence
QVQLVQSGAEVKKPGASVKMSCKASGYTFTSYTMHWVKQRPGQGLEWISYINPSSGYTKYNQKFKDRATLTADKSTSTAYMELSSLRSEDTAVYYCARWQDYDVYFDYWGQGTLVTVSS

SEQ ID NO: 52 - protein, 119 aa, Artificial
  CD3 VHH sequence
QVQLVQSGAEVKKPGASVKMSCKASGYTFTRYTMHWVKQAPGKGLEWISYINPSRGYTNYNQKFKDRATLTTDKSTSTAYMELSSLRSEDTAVYYCARYYDDHYCLDYWGQGTLVTVSS

SEQ ID NO: 53 - protein, 119 aa, Artificial
  CD3 VHH sequence
QVQLVQSGAEVKKPGASVKMSCKASGYTFTRYTMHWVKQRPGQGLEWIGYINPSRGYTNYNQKFKDRATLTTDKSTSTAYMELSSLRSEDTAVYYCARYYDDHYCLDYWGQGTLVTVSS

SEQ ID NO: 54 - protein, 119 aa, Artificial
  CD3 VH sequence
QVQLQQSGAELARPGASVKMSCKASGYTFTSYTMHWVKQRPGQGLEWIGYINPSSGYTKYNQKFKDKATLTADKSSSTAYMQLSSLTSEDSAVYYCARWQDYDVYFDYWGQGTTLTVSS

SEQ ID NO: 55 - protein, 106 aa, Artificial
  CD3 VL sequence
QIVLSQSPAILSASPGEKVTMTCRASSSVSYMHWYQQKPGSSPKPWIYDTSNLASGVPARFSGSGSGTSYSLTISRVEAEDAATYYCQQWSSNPPTFGGGTKLETK

SEQ ID NO: 56 - protein, 106 aa, Artificial
  CD3 VL sequence
QIVLSQSPAILSASPGEKVTMTCRASSSVSYMHWYQQKPGSSPKPWIYAASNRASGVPARFSGSGSGTSYSLTISRVEAEDAATYYCQQWSSNPPTFGGGTKLETK

SEQ ID NO: 57 - protein, 106 aa, Artificial
  CD3 VL sequence
QIVLSQSPAILSASPGEKVTMTCRASSSVSYMHWYQQKPGSSPKPWIYATSNRASGVPARFSGSGSGTSYSLTISRVEAEDAATYYCQQWSSNPPTFGGGTKLETK

SEQ ID NO: 58 - protein, 106 aa, Artificial
  CD3 VL sequence
QIVLSQSPAILSASPGEKVTMTCRASQSVSYMHWYQQKPGSSPKPWIYATSNRASGVPARFSGSGSGTSYSLTISRVEAEDAATYYCQQRSSNPPTFGGGTKLETK

SEQ ID NO: 59 - protein, 106 aa, Artificial
  CD3 VL sequence
QIVLSQSPAILSASPGEKVTMTCRASSSVSYMHWYQQKPGSSPKPWIYDTSNLASGVPARFSGSGSGTSYSLTISRVEAEDAATYYCQQRSSNPLTFGGGTKLETK

SEQ ID NO: 60 - protein, 106 aa, Artificial
  CD3 VL sequence
QIVLSQSPAILSASPGEKVTMTCRASQSVSYMHWYQQKPGSSPKPWIYDASNRASGVPARFSGSGSGTSYSLTISRVEAEDAATYYCQQWSSWPPTFGGGTKLETK

SEQ ID NO: 61 - protein, 15 aa, Artificial
  Fc hinge sequence
EPKSCDKTHTCPPCP

SEQ ID NO: 62 - protein, 15 aa, Artificial
  Fc hinge sequence
GGGGSDKTHTCPPCP

SEQ ID NO: 63 - protein, 15 aa, Artificial
  Fc hinge sequence
EPKSSDKTHTCPPCP

SEQ ID NO: 64 - protein, 10 aa, Artificial
  Fc hinge sequence
DKTHTCPPCP

SEQ ID NO: 65 - protein, 16 aa, Artificial
  Fc hinge sequence
STMVRSDKTHTCPPCP

SEQ ID NO: 66 - protein, 217 aa, Artificial
  Fc region sequence with knob mutations
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPYREEMTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 67 - protein, 217 aa, Artificial
  Fc region sequence hole mutations
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPEVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 68 - protein, 217 aa, Artificial
  Wildtype IgG1 Fc region
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 69 - protein, 98 aa, Artificial
  Heavy chain CH1 domain
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 70 - protein, 107 aa, Artificial [sequence omitted]

SEQ ID NO: 71 - protein, 5 aa, Artificial
  Linker peptide
GGGGS

SEQ ID NO: 72 - protein, 10 aa, Artificial
  Linker peptide
GGGGSGGGGS

SEQ ID NO: 73 - protein, 15 aa, Artificial
  Linker peptide
GGGGSGGGGSGGGGS

SEQ ID NO: 74 - protein, 356 aa, Artificial
  Exemplary chain 1 sequence of BCMA/CD3 BsAb in BiSpecific-V0 format
QVQLVESGGGLVQAGGSLRLSCASSGRLFRYTDMAWYRQAPGKQRELVAFSYVSGRTNYADSVKGRFTISRDNVKKVVYLQMSSLTPEDTAVYYCAQGRSPYRWGSYSSMFGSWGQGTLVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPYREEMTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 75 - protein, 356 aa, Artificial
  Exemplary chain 1 sequence of BCMA/CD3 BsAb in BiSpecific-V0 format
QVQLVESGGGLVQAGGSLRLSCASSGRLFRYTDMAWYRQAPGKQRELVAFSYVSGRTNYADSVKGRFTISRDNVKKVVYLQMSSLTPEDTAVYYCAQGRSPYRWGSYSSMFGSWGQGTLVTVSSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPYREEMTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 76 - protein, 351 aa, Artificial
  Exemplary chain 2 sequence of BCMA/CD3 BsAb in BiSpecific-V0 format
QVQLVQSGAEVKKPGASVKMSCKASGYTFTSYTMHWVKQRPGQGLEWISYINPSSGYTKYNQKFKDRATLTADKSTSTAYMELSSLRSEDTAVYYCARWQDYDVYFDYWGQGTLVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPEVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 77 - protein, 351 aa, Artificial
  Exemplary chain 2 sequence of BCMA/CD3 BsAb in BiSpecific-V0 format
QVQLVQSGAEVKKPGASVKMSCKASGYTFTSYTMHWVKQRPGQGLEWISYINPSSGYTKYNQKFKDRATLTADKSTSTAYMELSSLRSEDTAVYYCARWQDYDVYFDYWGQGTLVTVSSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPEVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 78 - protein, 485 aa, Artificial
  Exemplary chain 1 sequence of BCMA/CD3 TsAb in TriSpecific-V0 format
QVQLVESGGGLVQAGGSLRLSCTASGRTFGWYTMGWFRQGPGKEREFVAAISPSGGATYYADSVKGRFTISRDYAKNAVYLQMNSLKPEDTAVYYCNARLWGLGIRNYWGQGTLVTVSSGGGGSGGGGSQVQLVESGGGLVQAGGSLRLSCASSGRLFRYTDMAWYRQAPGKQRELVAFSYVSGRTNYADSVKGRFTISRDNVKKVVYLQMSSLTPEDTAVYYCAQGRSPYRWGSYSSMFGSWGQGTLVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPYREEMTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 79 - protein, 490 aa, Artificial
  Exemplary chain 1 sequence of BCMA/CD3 TsAb in TriSpecific-V0 format
QVQLVESGGGLVQAGGSLRLSCTASGRTFGWYTMGWFRQGPGKEREFVAAISPSGGATYYADSVKGRFTISRDYAKNAVYLQMNSLKPEDTAVYYCNARLWGLGIRNYWGQGTLVTVSSGGGGSGGGGSGGGGSQVQLVESGGGLVQAGGSLRLSCASSGRLFRYTDMAWYRQAPGKQRELVAFSYVSGRTNYADSVKGRFTISRDNVKKVVYLQMSSLTPEDTAVYYCAQGRSPYRWGSYSSMFGSWGQGTLVTVSSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPYREEMTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 80 - protein, 351 aa, Artificial; identical to SEQ ID NO: 76
  Exemplary chain 2 sequence of BCMA/CD3 TsAb in TriSpecific-V0 format

SEQ ID NO: 81 - protein, 351 aa, Artificial; identical to SEQ ID NO: 77
  Exemplary chain 2 sequence of BCMA/CD3 TsAb in TriSpecific-V0 format

SEQ ID NO: 82 - protein, 356 aa, Artificial; identical to SEQ ID NO: 74
  Exemplary chain 1 sequence of BCMA/CD3 BsAb in BiSpecific-V1 format

SEQ ID NO: 83 - protein, 356 aa, Artificial; identical to SEQ ID NO: 75
  Exemplary chain 1 sequence of BCMA/CD3 BsAb in BiSpecific-V1 format

SEQ ID NO: 84 - protein, 449 aa, Artificial
  Exemplary chain 2 sequence of BCMA/CD3 BsAb in BiSpecific-V1 format
QVQLQQSGAELARPGASVKMSCKASGYTFTSYTMHWVKQRPGQGLEWIGYINPSSGYTKYNQKFKDKATLTADKSSSTAYMQLSSLTSEDSAVYYCARWQDYDVYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPEVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 85 - protein, 449 aa, Artificial
  Exemplary chain 2 sequence of BCMA/CD3 BsAb in BiSpecific-V1 format
QVQLQQSGAELARPGASVKMSCKASGYTFTSYTMHWVKQRPGQGLEWIGYINPSSGYTKYNQKFKDKATLTADKSSSTAYMQLSSLTSEDSAVYYCARWQDYDVYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPEVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 86 - protein, 213 aa, Artificial
  Exemplary chain 3 sequence of BCMA/CD3 BsAb in BiSpecific-V1 format
QIVLSQSPAILSASPGEKVTMTCRASSSVSYMHWYQQKPGSSPKPWIYAASNRASGVPARFSGSGSGTSYSLTISRVEAEDAATYYCQQWSSNPPTFGGGTKLETKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 87 - protein, 485 aa, Artificial; identical to SEQ ID NO: 78
  Exemplary chain 1 sequence of BCMA/CD3 TsAb in TriSpecific-V1 format

SEQ ID NO: 88 - protein, 490 aa, Artificial; identical to SEQ ID NO: 79
  Exemplary chain 1 sequence of BCMA/CD3 TsAb in TriSpecific-V1 format

SEQ ID NO: 89 - protein, 449 aa, Artificial; identical to SEQ ID NO: 84
  Exemplary chain 2 sequence of BCMA/CD3 TsAb in TriSpecific-V1 format

SEQ ID NO: 90 - protein, 449 aa, Artificial; identical to SEQ ID NO: 85
  Exemplary chain 2 sequence of BCMA/CD3 TsAb in TriSpecific-V1 format

SEQ ID NO: 91 - protein, 213 aa, Artificial; identical to SEQ ID NO: 86
  Exemplary chain 3 sequence of BCMA/CD3 TsAb in TriSpecific-V1 format

SEQ ID NO: 92 - protein, 356 aa, Artificial; identical to SEQ ID NO: 74
  Exemplary chain 1 sequence of BCMA/CD3 BsAb in BiSpecific-V2 format

SEQ ID NO: 93 - protein, 356 aa, Artificial; identical to SEQ ID NO: 75
  Exemplary chain 1 sequence of BCMA/CD3 BsAb in BiSpecific-V2 format

SEQ ID NO: 94 - protein, 472 aa, Artificial
  Exemplary chain 2 sequence of BCMA/CD3 BsAb in BiSpecific-V2 format
QVQLQQSGAELARPGASVKMSCKASGYTFTSYTMHWVKQRPGQGLEWIGYINPSSGYTKYNQKFKDKATLTADKSSSTAYMQLSSLTSEDSAVYYCARWQDYDVYFDYWGQGTTLTVSSGGGGSGGGGSGGGGSQIVLSQSPAILSASPGEKVTMTCRASSSVSYMHWYQQKPGSSPKPWIYAASNRASGVPARFSGSGSGTSYSLTISRVEAEDAATYYCQQWSSNPPTFGGGTKLETKGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPEVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 95 - protein, 472 aa, Artificial
  Exemplary chain 2 sequence of BCMA/CD3 BsAb in BiSpecific-V2 format
QVQLQQSGAELARPGASVKMSCKASGYTFTSYTMHWVKQRPGQGLEWIGYINPSSGYTKYNQKFKDKATLTADKSSSTAYMQLSSLTSEDSAVYYCARWQDYDVYFDYWGQGTTLTVSSGGGGSGGGGSGGGGSQIVLSQSPAILSASPGEKVTMTCRASSSVSYMHWYQQKPGSSPKPWIYAASNRASGVPARFSGSGSGTSYSLTISRVEAEDAATYYCQQWSSNPPTFGGGTKLETKEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPEVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 96 - protein, 485 aa, Artificial; identical to SEQ ID NO: 78
  Exemplary chain 1 sequence of BCMA/CD3 TsAb in TriSpecific-V2 format

SEQ ID NO: 97 - protein, 490 aa, Artificial; identical to SEQ ID NO: 79
  Exemplary chain 1 sequence of BCMA/CD3 TsAb in TriSpecific-V2 format

SEQ ID NO: 98 - protein, 472 aa, Artificial; identical to SEQ ID NO: 94
  Exemplary chain 2 sequence of BCMA/CD3 TsAb in TriSpecific-V2 format

SEQ ID NO: 99 - protein, 472 aa, Artificial; identical to SEQ ID NO: 95
  Exemplary chain 2 sequence of BCMA/CD3 TsAb in TriSpecific-V2 format

SEQ ID NO: 100 - protein, 588 aa, Artificial
  Exemplary chain 1 sequence of BCMA/CD3 BsAb in BiSpecific-V3 format
QVQLQQSGAELARPGASVKMSCKASGYTFTSYTMHWVKQRPGQGLEWIGYINPSSGYTKYNQKFKDKATLTADKSSSTAYMQLSSLTSEDSAVYYCARWQDYDVYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSQVQLVESGGGLVQAGGSLRLSCASSGRLFRYTDMAWYRQAPGKQRELVAFSYVSGRTNYADSVKGRFTISRDNVKKVVYLQMSSLTPEDTAVYYCAQGRSPYRWGSYSSMFGSWGQGTLVTVSS

SEQ ID NO: 101 - protein, 583 aa, Artificial
  Exemplary chain 1 sequence of BCMA/CD3 BsAb in BiSpecific-V3 format
QVQLQQSGAELARPGASVKMSCKASGYTFTSYTMHWVKQRPGQGLEWIGYINPSSGYTKYNQKFKDKATLTADKSSSTAYMQLSSLTSEDSAVYYCARWQDYDVYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSQVQLVESGGGLVQAGGSLRLSCASSGRLFRYTDMAWYRQAPGKQRELVAFSYVSGRTNYADSVKGRFTISRDNVKKVVYLQMSSLTPEDTAVYYCAQGRSPYRWGSYSSMFGSWGQGTLVTVSS

SEQ ID NO: 102 - protein, 213 aa, Artificial; identical to SEQ ID NO: 86
  Exemplary chain 2 sequence of BCMA/CD3 BsAb in BiSpecific-V3 format

SEQ ID NO: 103 - protein, 588 aa, Artificial
  Exemplary chain 1 sequence of BCMA/CD3 TsAb in TriSpecific-V3 format
QVQLQQSGAELARPGASVKMSCKASGYTFTSYTMHWVKQRPGQGLEWIGYINPSSGYTKYNQKFKDKATLTADKSSSTAYMQLSSLTSEDSAVYYCARWQDYDVYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPYREEMTKNQVSLYCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSQVQLVESGGGLVQAGGSLRLSCASSGRLFRYTDMAWYRQAPGKQRELVAFSYVSGRTNYADSVKGRFTISRDNVKKVVYLQMSSLTPEDTAVYYCAQGRSPYRWGSYSSMFGSWGQGTLVTVSS

SEQ ID NO: 104 - protein, 583 aa, Artificial
  Exemplary chain 1 sequence of BCMA/CD3 TsAb in TriSpecific-V3 format
QVQLQQSGAELARPGASVKMSCKASGYTFTSYTMHWVKQRPGQGLEWIGYINPSSGYTKYNQKFKDKATLTADKSSSTAYMQLSSLTSEDSAVYYCARWQDYDVYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPYREEMTKNQVSLYCLVKGFYPSDIAVEWESNGQPENNYKTTPPVLDSDGSFFLYSKLTVDKSRWQQGNVFSCSVMHEALHNHYTQKSLSLSPGKGGGGSGGGGSQVQLVESGGGLVQAGGSLRLSCASSGRLFRYTDMAWYRQAPGKQRELVAFSYVSGRTNYADSVKGRFTISRDNVKKVVYLQMSSLTPEDTAVYYCAQGRSPYRWGSYSSMFGSWGQGTLVTVSS

SEQ ID NO: 105 - protein, 583 aa, Artificial
  Exemplary chain 2 sequence of BCMA/CD3 TsAb in TriSpecific-V3 format
QVQLQQSGAELARPGASVKMSCKASGYTFTSYTMHWVKQRPGQGLEWIGYINPSSGYTKYNQKFKDKATLTADKSSSTAYMQLSSLTSEDSAVYYCARWQDYDVYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPEVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGKGGGGSGGGGSGGGGSQVQLVESGGGLVQAGGSLRLSCTASGRTFGWYTMGWFRQGPGKEREFVAAISPSGGATYYADSVKGRFTISRDYAKNAVYLQMNSLKPEDTAVYYCNARLWGLGIRNYWGQGTLVTVSS

SEQ ID NO: 106 - protein, 578 aa, Artificial
  Exemplary chain 2 sequence of BCMA/CD3 TsAb in TriSpecific-V3 format
QVQLQQSGAELARPGASVKMSCKASGYTFTSYTMHWVKQRPGQGLEWIGYINPSSGYTKYNQKFKDKATLTADKSSSTAYMQLSSLTSEDSAVYYCARWQDYDVYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPEVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGKGGGGSGGGGSQVQLVESGGGLVQAGGSLRLSCTASGRTFGWYTMGWFRQGPGKEREFVAAISPSGGATYYADSVKGRFTISRDYAKNAVYLQMNSLKPEDTAVYYCNARLWGLGIRNYWGQGTLVTVSS

SEQ ID NO: 107 - protein, 213 aa, Artificial; identical to SEQ ID NO: 86
  Exemplary chain 3 sequence of BCMA/CD3 TsAb in TriSpecific-V3 format

SEQ ID NO: 108 - protein, 588 aa, Artificial
  Exemplary chain 1 sequence of BCMA/CD3 BsAb in BiSpecific-V4 format
QVQLVESGGGLVQAGGSLRLSCASSGRLFRYTDMAWYRQAPGKQRELVAFSYVSGRTNYADSVKGRFTISRDNVKKVVYLQMSSLTPEDTAVYYCAQGRSPYRWGSYSSMFGSWGQGTLVTVSSGGGGSGGGGSGGGGSQVQLQQSGAELARPGASVKMSCKASGYTFTSYTMHWVKQRPGQGLEWIGYINPSSGYTKYNQKFKDKATLTADKSSSTAYMQLSSLTSEDSAVYYCARWQDYDVYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 109 - protein, 583 aa, Artificial
  Exemplary chain 1 sequence of BCMA/CD3 BsAb in BiSpecific-V4 format
QVQLVESGGGLVQAGGSLRLSCASSGRLFRYTDMAWYRQAPGKQRELVAFSYVSGRTNYADSVKGRFTISRDNVKKVVYLQMSSLTPEDTAVYYCAQGRSPYRWGSYSSMFGSWGQGTLVTVSSGGGGSGGGGSQVQLQQSGAELARPGASVKMSCKASGYTFTSYTMHWVKQRPGQGLEWIGYINPSSGYTKYNQKFKDKATLTADKSSSTAYMQLSSLTSEDSAVYYCARWQDYDVYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 110 - protein, 213 aa, Artificial; identical to SEQ ID NO: 86
  Exemplary chain 2 sequence of BCMA/CD3 BsAb in BiSpecific-V4 format

SEQ ID NO: 111 - protein, 588 aa, Artificial
  Exemplary chain 1 sequence of BCMA/CD3 TsAb in TriSpecific-V4 format
QVQLVESGGGLVQAGGSLRLSCASSGRLFRYTDMAWYRQAPGKQRELVAFSYVSGRTNYADSVKGRFTISRDNVKKVVYLQMSSLTPEDTAVYYCAQGRSPYRWGSYSSMFGSWGQGTLVTVSSGGGGSGGGGSGGGGSQVQLQQSGAELARPGASVKMSCKASGYTFTSYTMHWVKQRPGQGLEWIGYINPSSGYTKYNQKFKDKATLTADKSSSTAYMQLSSLTSEDSAVYYCARWQDYDVYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPYREEMTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 112 - protein, 583 aa, Artificial
  Exemplary chain 1 sequence of BCMA/CD3 TsAb in TriSpecific-V4 format
QVQLVESGGGLVQAGGSLRLSCASSGRLFRYTDMAWYRQAPGKQRELVAFSYVSGRTNYADSVKGRFTISRDNVKKVVYLQMSSLTPEDTAVYYCAQGRSPYRWGSYSSMFGSWGQGTLVTVSSGGGGSGGGGSQVQLQQSGAELARPGASVKMSCKASGYTFTSYTMHWVKQRPGQGLEWIGYINPSSGYTKYNQKFKDKATLTADKSSSTAYMQLSSLTSEDSAVYYCARWQDYDVYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPYREEMTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 113 - protein, 583 aa, Artificial
  Exemplary chain 2 sequence of BCMA/CD3 TsAb in TriSpecific-V4 format
QVQLVESGGGLVQAGGSLRLSCTASGRTFGWYTMGWFRQGPGKEREFVAAISPSGGATYYADSVKGRFTISRDYAKNAVYLQMNSLKPEDTAVYYCNARLWGLGIRNYWGQGTLVTVSSGGGGSGGGGSGGGGSQVQLQQSGAELARPGASVKMSCKASGYTFTSYTMHWVKQRPGQGLEWIGYINPSSGYTKYNQKFKDKATLTADKSSSTAYMQLSSLTSEDSAVYYCARWQDYDVYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPEVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 114 - protein, 578 aa, Artificial
  Exemplary chain 2 sequence of BCMA/CD3 TsAb in TriSpecific-V4 format
QVQLVESGGGLVQAGGSLRLSCTASGRTFGWYTMGWFRQGPGKEREFVAAISPSGGATYYADSVKGRFTISRDYAKNAVYLQMNSLKPEDTAVYYCNARLWGLGIRNYWGQGTLVTVSSGGGGSGGGGSQVQLQQSGAELARPGASVKMSCKASGYTFTSYTMHWVKQRPGQGLEWIGYINPSSGYTKYNQKFKDKATLTADKSSSTAYMQLSSLTSEDSAVYYCARWQDYDVYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPEVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 115 - protein, 213 aa, Artificial; identical to SEQ ID NO: 86
  Exemplary chain 3 sequence of BCMA/CD3 TsAb in TriSpecific-V4 format

SEQ ID NO: 116 - protein, 355 aa, Artificial
  Exemplary chain 1 sequence of CD38/CD3 BsAb in BiSpecific-V0 format
EVQLVESGGGLVQAGGSLRLSCAVSGTVGPTVMGWFRQAPGKKREFVALIGWTGGVTAYADSVKDRFTISRDCAENRVDLQMDSLKPEDTAVYYCAADSNRRRYYGPYEYDSWGQGTLVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPYREEMTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 117 - protein, 355 aa, Artificial
  Exemplary chain 1 sequence of CD38/CD3 BsAb in BiSpecific-V0 format
EVQLVESGGGLVQAGGSLRLSCAVSGTVGPTVMGWFRQAPGKKREFVALIGWTGGVTAYADSVKDRFTISRDCAENRVDLQMDSLKPEDTAVYYCAADSNRRRYYGPYEYDSWGQGTLVTVSSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPYREEMTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 118 - protein, 351 aa, Artificial; identical to SEQ ID NO: 76
  Exemplary chain 2 sequence of CD38/CD3 BsAb in BiSpecific-V0 format

SEQ ID NO: 119 - protein, 351 aa, Artificial; identical to SEQ ID NO: 77
  Exemplary chain 2 sequence of CD38/CD3 BsAb in BiSpecific-V0 format

SEQ ID NO: 120 - protein, 355 aa, Artificial; identical to SEQ ID NO: 116
  Exemplary chain 1 sequence of CD38/CD3 BsAb in BiSpecific-V2 format

SEQ ID NO: 121 - protein, 355 aa, Artificial; identical to SEQ ID NO: 117
  Exemplary chain 1 sequence of CD38/CD3 BsAb in BiSpecific-V2 format

SEQ ID NO: 122 - protein, 472 aa, Artificial; identical to SEQ ID NO: 94
  Exemplary chain 2 sequence of CD38/CD3 BsAb in BiSpecific-V2 format

SEQ ID NO: 123 - protein, 472 aa, Artificial; identical to SEQ ID NO: 95
  Exemplary chain 2 sequence of CD38/CD3 BsAb in BiSpecific-V2 format

SEQ ID NO: 124 - protein, 20 aa, Artificial
  Linker peptide
GGGGSGGGGSGGGGSGGGGS

SEQ ID NO: 125 - protein, 24 aa, Artificial
  Linker peptide
GGGGSGGGGSGGGGSGGGGSGGGG

SEQ ID NO: 126 - protein, 30 aa, Artificial
  Linker peptide
GGGGSGGGGSGGGGSGGGGSGGGGSGGGGS

(19) United States
(12) Patent Application Publication
(10) Pub. No.: US 2024/0002539 A1
(43) Pub. Date: Jan. 4, 2024

(54) MULTISPECIFIC ANTIBODIES AND USES THEREOF

(71) Applicant: Ab Studio Inc., Hayward, CA (US)

(72) Inventors: Yanan Wang, Hayward, CA (US); Yue Liu, Foster City, CA (US); Jianbo Dong, Santa Clara, CA (US); Chao Bai Huang, San Leandro, CA (US); Allison Titong, Hayward, CA (US); Bo Wang, San Carlos, CA (US); Sachith Gallolu Kankanamalage, Hayward, CA (US)

(21) Appl. No.: 18/035,027

(22) PCT Filed: Nov. 3, 2021
(86) PCT No.: PCT/US2021/057916

§ 371 Date: May 2, 2023

Related U.S. Application Data
(60) Provisional application No. 63/109,317, filed on Nov. 3, 2020.

(51) Int. Cl. C07K 16/46; C12N 15/63; A61P 35/00

(52) U.S. Cl. CPC C07K 16/468; C12N 15/63; A61P 35/00; C07K 2317/31; C07K 2317/565; C07K 2317/52; C07K 2317/24; C07K 2317/622; C07K 2317/14

(57) ABSTRACT

This disclosure relates to multispecific antibodies (e.g., bispecific antibodies) or antigen-binding fragments thereof. In one aspect, the multispecific antibodies or antigen-binding fragments thereof binds to CD3, BCMA, and/or CD38, or a combination thereof.

### TECHNICAL FIELD

This disclosure relates to multispecific antibodies or antigen-binding fragments thereof.

### BACKGROUND

A multispecific antibody is an artificial protein that can simultaneously bind to two or more different epitopes. This opens up a wide range of applications, including redirecting T cells to tumor cells, blocking two different signaling pathways simultaneously, dual targeting of different disease mediators, and delivering payloads to targeted sites. The approval of catumaxomab (anti-EpCAM and anti-CD3) and blinatumomab (anti-CD19 and anti-CD3) has become a major milestone in the development of multispecific antibodies.

As multispecific antibodies have various applications, there is a need to continue to develop various therapeutics based on multispecific antibodies.

### SUMMARY

This disclosure relates to antibodies or antigen-binding fragments, wherein the antibodies or antigen-binding fragments specifically bind to CD3, BCMA, and/or CD38, or a combination thereof.

In one aspect, the disclosure is related to an antibody or antigen-binding fragment thereof that binds to BCMA (B-cell maturation antigen), comprising: a heavy-chain antibody variable domain (VHH) comprising complementarity determining regions (CDRs) 1, 2, and 3. In some embodiments, the VHH CDR1 region comprises an amino acid sequence that is at least 80% identical to a selected VHH CDR1 amino acid sequence, the VHH CDR2 region comprises an amino acid sequence that is at least 80% identical to a selected VHH CDR2 amino acid sequence, and the VHH CDR3 region comprises an amino acid sequence that is at least 80% identical to a selected VHH CDR3 amino acid sequence. In some embodiments, the selected VHH CDRs 1, 2, and 3 amino acid sequences are one of the following:
(1) the selected VHH CDRs 1, 2, 3 amino acid sequences are set forth in SEQ ID NOs: 1, 2, and 3, respectively;
(2) the selected VHH CDRs 1, 2, 3 amino acid sequences are set forth in SEQ ID NOs: 4, 5, and 6, respectively;
(3) the selected VHH CDRs 1, 2, 3 amino acid sequences are set forth in SEQ ID NOs: 7, 8, and 9, respectively; and
(4) the selected VHH CDRs 1, 2, 3 amino acid sequences are set forth in SEQ ID NOs: 10, 11, and 12, respectively.

In some embodiments, the VHH comprises CDRs 1, 2, 3 with the amino acid sequences set forth in SEQ ID NOs: 1, 2, and 3, respectively. In some embodiments, the VHH comprises CDRs 1, 2, 3 with the amino acid sequences set forth in SEQ ID NOs: 4, 5, and 6, respectively. In some embodiments, the VHH comprises CDRs 1, 2, 3 with the amino acid sequences set forth in SEQ ID NOs: 7, 8, and 9, respectively. In some embodiments, the VHH comprises CDRs 1, 2, 3 with the amino acid sequences set forth in SEQ ID NOs: 10, 11, and 12, respectively.

In one aspect, the disclosure is related to an antibody or antigen-binding fragment thereof that binds to BCMA comprising a heavy-chain antibody variable domain (VHH) comprising an amino acid sequence that is at least 80% identical to a selected VHH sequence. In some embodiments, the selected VHH sequence is selected from the group consisting of SEQ ID NOs: 46-49.

In some embodiments, the VHH comprises the sequence of SEQ ID NO: 46. In some embodiments, the VHH comprises the sequence of SEQ ID NO: 47. In some embodiments, the VHH comprises the sequence of SEQ ID NO: 48. In some embodiments, the VHH comprises the sequence of SEQ ID NO: 49.

In some embodiments, the antibody or antigen-binding fragment specifically binds to BCMA. In some embodiments, the antibody or antigen-binding fragment is a humanized antibody or antigen-binding fragment thereof.

In one aspect, the disclosure is related to an antibody or antigen-binding fragment thereof comprising the VHH CDRs 1, 2, 3, of the antibody or antigen-binding fragment thereof as described herein.

In some embodiments, the antibody or antigen-binding fragment comprises a human IgG Fc.

In some embodiments, the antibody or antigen-binding fragment comprises two or more heavy-chain antibody variable domains.

In one aspect, the disclosure is related to an antibody or antigen-binding fragment thereof that cross-competes with the antibody or antigen-binding fragment thereof as described herein.

In one aspect, the disclosure is related to an antibody or antigen-binding fragment thereof that binds to CD38 (cluster of differentiation 38), comprising: a heavy-chain antibody variable domain (VHH) comprising complementarity determining regions (CDRs) 1, 2, and 3. In some embodiments, the VHH CDR1 region comprises an amino acid sequence that is at least 80% identical to SEQ ID NO: 13, the VHH CDR2 region comprises an amino acid sequence that is at least 80% identical to SEQ ID NO: 14, and the VHH CDR3 region comprises an amino acid sequence that is at least 80% identical to SEQ ID NO: 15.

In one aspect, the disclosure is related to an antibody or antigen-binding fragment thereof that binds to CD38 comprising a heavy-chain antibody variable domain (VHH) comprising an amino acid sequence that is at least 80% identical to SEQ ID NO: 50.

In some embodiments, the antibody or antigen-binding fragment specifically binds to CD38. In some embodiments, the antibody or antigen-binding fragment is a humanized antibody or antigen-binding fragment thereof.

In one aspect, the disclosure is related to an antibody or antigen-binding fragment thereof comprising the VHH CDRs 1, 2, 3, of the antibody or antigen-binding fragment thereof as described herein.

In some embodiments, the antibody or antigen-binding fragment comprises a human IgG Fc.

In some embodiments, the antibody or antigen-binding fragment comprises two or more heavy-chain antibody variable domains.

In one aspect, the disclosure is related to an antibody or antigen-binding fragment thereof that cross-competes with the antibody or antigen-binding fragment thereof as described herein.

In one aspect, the disclosure is related to an antibody or antigen-binding fragment thereof that binds to CD3 (cluster of differentiation 3), comprising a heavy-chain antibody variable domain (VHH) comprising complementarity determining regions (CDRs) 1, 2, and 3. In some embodiments, the VHH CDR1 region comprises an amino acid sequence that is at least 80% identical to a selected VHH CDR1 amino acid sequence, the VHH CDR2 region comprises an amino acid sequence that is at least 80% identical to a selected VHH CDR2 amino acid sequence, and the VHH CDR3 region comprises an amino acid sequence that is at least 80% identical to a selected VHH CDR3 amino acid sequence. In some embodiments, the selected VHH CDRs 1, 2, and 3 amino acid sequences are one of the following:
(1) the selected VHH CDRs 1, 2, 3 amino acid sequences are set forth in SEQ ID NOs: 16, 17, and 18, respectively;
(2) the selected VHH CDRs 1, 2, 3 amino acid sequences are set forth in SEQ ID NOs: 19, 20, and 21, respectively; and
(3) the selected VHH CDRs 1, 2, 3 amino acid sequences are set forth in SEQ ID NOs: 22, 23, and 24, respectively.

In some embodiments, the VHH comprises CDRs 1, 2, 3 with the amino acid sequences set forth in SEQ ID NOs: 16, 17, and 18, respectively. In some embodiments, the VHH comprises CDRs 1, 2, 3 with the amino acid sequences set forth in SEQ ID NOs: 19, 20, and 21, respectively. In some embodiments, the VHH comprises CDRs 1, 2, 3 with the amino acid sequences set forth in SEQ ID NOs: 22, 23, and 24, respectively.

In one aspect, the disclosure is related to an antibody or antigen-binding fragment thereof that binds to CD3 comprising a heavy-chain antibody variable domain (VHH) comprising an amino acid sequence that is at least 80% identical to a selected VHH sequence. In some embodiments, the selected VHH sequence is selected from the group consisting of SEQ ID NOs: 51-53.

In some embodiments, the VHH comprises the sequence of SEQ ID NO: 51. In some embodiments, the VHH comprises the sequence of SEQ ID NO: 52. In some embodiments, the VHH comprises the sequence of SEQ ID NO: 53.

In some embodiments, the antibody or antigen-binding fragment specifically binds to CD3. In some embodiments, the antibody or antigen-binding fragment is a humanized antibody or antigen-binding fragment thereof.

In one aspect, the disclosure is related to an antibody or antigen-binding fragment thereof comprising the VHH CDRs 1, 2, 3, of the antibody or antigen-binding fragment thereof as described herein.

In some embodiments, the antibody or antigen-binding fragment comprises a human IgG Fc.

In some embodiments, the antibody or antigen-binding fragment comprises two or more heavy-chain antibody variable domains.

In one aspect, the disclosure is related to an antibody or antigen-binding fragment thereof that cross-competes with the antibody or antigen-binding fragment thereof as described herein.

In one aspect, the disclosure is related to an antibody or antigen-binding fragment thereof that binds to CD3 (cluster of differentiation 3) comprising a heavy chain variable region (VH) comprising complementarity determining regions (CDRs) 1, 2, and 3, and a light chain variable region (VL) comprising CDRs 1, 2, and 3. In some embodiments, the VH CDR1 region comprises an amino acid sequence that is at least 80% identical to a selected VH CDR1 amino acid sequence, the VH CDR2 region comprises an amino acid sequence that is at least 80% identical to a selected VH CDR2 amino acid sequence, and the VH CDR3 region comprises an amino acid sequence that is at least 80% identical to a selected VH CDR3 amino acid sequence. In some embodiments, the VL CDR1 region comprises an amino acid sequence that is at least 80% identical to a selected VL CDR1 amino acid sequence, the VL CDR2 region comprises an amino acid sequence that is at least 80% identical to a selected VL CDR2 amino acid sequence, and the VL CDR3 region comprises an amino acid sequence that is at least 80% identical to a selected VL CDR3 amino acid sequence. In some embodiments, the selected VH CDRs 1, 2, and 3 amino acid sequences and the selected VL CDRs, 1, 2, and 3 amino acid sequences are one of the following:
(1) the selected VH CDRs 1, 2, 3 amino acid sequences are set forth in SEQ ID NOs: 25, 26, and 27, respectively, and the selected VL CDRs 1, 2, 3 amino acid sequences are set forth in SEQ ID NOs: 28, 29, and 30, respectively;
(2) the selected VH CDRs 1, 2, 3 amino acid sequences are set forth in SEQ ID NOs: 25, 26, and 27, respectively, and the selected VL CDRs 1, 2, 3 amino acid sequences are set forth in SEQ ID NOs: 31, 32, and 33, respectively;
(3) the selected VH CDRs 1, 2, 3 amino acid sequences are set forth in SEQ ID NOs: 25, 26, and 27, respectively, and the selected VL CDRs 1, 2, 3 amino acid sequences are set forth in SEQ ID NOs: 34, 35, and 36, respectively;
(4) the selected VH CDRs 1, 2, 3 amino acid sequences are set forth in SEQ ID NOs: 25, 26, and 27, respectively, and the selected VL CDRs 1, 2, 3 amino acid sequences are set forth in SEQ ID NOs: 37, 38, and 39, respectively;
(5) the selected VH CDRs 1, 2, 3 amino acid sequences are set forth in SEQ ID NOs: 25, 26, and 27, respectively, and the selected VL CDRs 1, 2, 3 amino acid sequences are set forth in SEQ ID NOs: 40, 41, and 42, respectively; and
(6) the selected VH CDRs 1, 2, 3 amino acid sequences are set forth in SEQ ID NOs: 25, 26, and 27, respectively, and the selected VL CDRs 1, 2, 3 amino acid sequences are set forth in SEQ ID NOs: 43, 44, and 45, respectively.

In some embodiments, the VH comprises CDRs 1, 2, 3 with the amino acid sequences set forth in SEQ ID NOs: 25, 26, and 27, respectively, and the VL comprises CDRs 1, 2, 3 with the amino acid sequences set forth in SEQ ID NOs: 28, 29, and 30, respectively.

In some embodiments, the VH comprises CDRs 1, 2, 3 with the amino acid sequences set forth in SEQ ID NOs: 25, 26, and 27, respectively, and the VL comprises CDRs 1, 2, 3 with the amino acid sequences set forth in SEQ ID NOs: 31, 32, and 33, respectively.

In some embodiments, the VH comprises CDRs 1, 2, 3 with the amino acid sequences set forth in SEQ ID NOs: 25, 26, and 27, respectively, and the VL comprises CDRs 1, 2, 3 with the amino acid sequences set forth in SEQ ID NOs: 34, 35, and 36, respectively.

In some embodiments, the VH comprises CDRs 1, 2, 3 with the amino acid sequences set forth in SEQ ID NOs: 25, 26, and 27, respectively, and the VL comprises CDRs 1, 2, 3 with the amino acid sequences set forth in SEQ ID NOs: 37, 38, and 39, respectively.

In some embodiments, the VH comprises CDRs 1, 2, 3 with the amino acid sequences set forth in SEQ ID NOs: 25, 26, and 27, respectively, and the VL comprises CDRs 1, 2, 3 with the amino acid sequences set forth in SEQ ID NOs: 40, 41, and 42, respectively.

In some embodiments, the VH comprises CDRs 1, 2, 3 with the amino acid sequences set forth in SEQ ID NOs: 25, 26, and 27, respectively, and the VL comprises CDRs 1, 2, 3 with the amino acid sequences set forth in SEQ ID NOs: 43, 44, and 45, respectively.

In one aspect, the disclosure is related to an antibody or antigen-binding fragment thereof that binds to CD3 comprising a heavy chain variable region (VH) comprising an amino acid sequence that is at least 90% identical to SEQ ID NO: 54, and a light chain variable region (VL) comprising an amino acid sequence that is at least 90% identical to SEQ ID NO: 55, 56, 57, 58, 59, or 60.

In some embodiments, the VH comprises the sequence of SEQ ID NO: 54 and the VL comprises the sequence of SEQ ID NO: 55.

In some embodiments, the VH comprises the sequence of SEQ ID NO: 54 and the VL comprises the sequence of SEQ ID NO: 56.

In some embodiments, the VH comprises the sequence of SEQ ID NO: 54 and the VL comprises the sequence of SEQ ID NO: 57.

In some embodiments, the VH comprises the sequence of SEQ ID NO: 54 and the VL comprises the sequence of SEQ ID NO: 58.

In some embodiments, the VH comprises the sequence of SEQ ID NO: 54 and the VL comprises the sequence of SEQ ID NO: 59.

In some embodiments, the VH comprises the sequence of SEQ ID NO: 54 and the VL comprises the sequence of SEQ ID NO: 60.

In some embodiments, the antibody or antigen-binding fragment specifically binds to human CD3. In some embodiments, the antibody or antigen-binding fragment is a single-chain variable fragment (scFV).

In one aspect, the disclosure is related to an antibody or antigen-binding fragment thereof comprising the VH CDRs 1, 2, 3, and VL CDRs 1, 2, 3, of the antibody or antigen-binding fragment thereof as described herein.

In one aspect, the disclosure is related to an antibody or antigen-binding fragment thereof that cross-competes with the antibody or antigen-binding fragment thereof as described herein.

In some embodiments, the antibody is a bispecific antibody or a multispecific antibody.

In one aspect, the disclosure is related to a multi-specific antibody or antigen-binding fragment thereof, comprising a first antigen-binding site that specifically binds to CD3, and a second antigen-binding site that specifically binds to BCMA or CD38. In some embodiments, the multi-specific antibody or antigen-binding fragment thereof described herein further comprises a third antigen-binding site that specifically binds to BCMA or CD38. In some embodiments,
a) the second antigen-binding site that specifically binds to BCMA and the third antigen-binding site that specifically binds to BCMA;
b) the second antigen-binding site that specifically binds to BCMA and the third antigen-binding site that specifically binds to CD38; or
c) the second antigen-binding site that specifically binds to CD38 and the third antigen-binding site that specifically binds to CD38.

In some embodiments, the first antigen-binding site comprises a first heavy-chain antibody variable domain (VHH1) comprising complementarity determining regions (CDRs) 1, 2, and 3. In some embodiments, the VHH1 CDR1 region comprises an amino acid sequence that is at least 80% identical to a selected VHH1 CDR1 amino acid sequence, the VHH1 CDR2 region comprises an amino acid sequence that is at least 80% identical to a selected VHH1 CDR2 amino acid sequence, and the VHH1 CDR3 region comprises an amino acid sequence that is at least 80% identical to a selected VHH1 CDR3 amino acid sequence. In some embodiments, the selected VHH1 CDRs 1, 2, and 3 amino acid sequences are one of the following:
(1) the selected VHH1 CDRs 1, 2, 3 amino acid sequences are set forth in SEQ ID NOs: 16, 17, and 18, respectively;
(2) the selected VHH1 CDRs 1, 2, 3 amino acid sequences are set forth in SEQ ID NOs: 19, 20, and 21, respectively; and
(3) the selected VHH1 CDRs 1, 2, 3 amino acid sequences are set forth in SEQ ID NOs: 22, 23, and 24, respectively.

In some embodiments, the first heavy-chain antibody variable domain (VHH1) comprises an amino acid sequence that is at least 80% identical to a selected VHH1 sequence. In some embodiments, the selected VHH1 sequence is selected from the group consisting of SEQ ID NOs: 51-53.

In some embodiments, the second antigen-binding site specifically binds to BCMA, and the second antigen-binding site comprises a second heavy-chain antibody variable domain (VHH2) comprising complementarity determining regions (CDRs) 1, 2, and 3. In some embodiments, the VHH2 CDR1 region comprises an amino acid sequence that is at least 80% identical to a selected VHH2 CDR1 amino acid sequence, the VHH2 CDR2 region comprises an amino acid sequence that is at least 80% identical to a selected VHH2 CDR2 amino acid sequence, and the VHH2 CDR3 region comprises an amino acid sequence that is at least 80% identical to a selected VHH2 CDR3 amino acid sequence. In some embodiments, the selected VHH2 CDRs 1, 2, and 3 amino acid sequences are one of the following:
(1) the selected VHH2 CDRs 1, 2, 3 amino acid sequences are set forth in SEQ ID NOs: 1, 2, and 3, respectively;
(2) the selected VHH2 CDRs 1, 2, 3 amino acid sequences are set forth in SEQ ID NOs: 4, 5, and 6, respectively;
(3) the selected VHH2 CDRs 1, 2, 3 amino acid sequences are set forth in SEQ ID NOs: 7, 8, and 9, respectively; and
(4) the selected VHH2 CDRs 1, 2, 3 amino acid sequences are set forth in SEQ ID NOs: 10, 11, and 12, respectively.

In some embodiments, the second heavy-chain antibody variable domain (VHH2) comprises an amino acid sequence that is at least 80% identical to a selected VHH2 sequence. In some embodiments, the selected VHH2 sequence is selected from the group consisting of SEQ ID NOs: 46-49.

In some embodiments, the second antigen-binding site specifically binds to CD38. In some embodiments, the second antigen-binding site comprises a second heavy-chain antibody variable domain (VHH2) comprising complementarity determining regions (CDRs) 1, 2, and 3. In some embodiments, the VHH2 CDR1 region comprises an amino acid sequence that is at least 80% identical to SEQ ID NO: 13, the VHH2 CDR2 region comprises an amino acid sequence that is at least 80% identical to SEQ ID NO: 14, and the VHH2 CDR3 region comprises an amino acid sequence that is at least 80% identical to SEQ ID NO: 15.

In some embodiments, the second heavy-chain antibody variable domain (VHH2) comprises an amino acid sequence that is at least 80% identical to SEQ ID NO: 50.

In some embodiments, the first antigen-binding site comprises a heavy chain variable region (VH) and a light chain variable region (VL). In some embodiments, the VH and the VL associate with each other and specifically bind to CD3. In some embodiments, the heavy chain variable region (VH) comprising complementarity determining regions (CDRs) 1, 2, and 3, and the light chain variable region (VL) comprising CDRs 1, 2, and 3. In some embodiments, the VH CDR1 region comprises an amino acid sequence that is at least 80% identical to a selected VH CDR1 amino acid sequence, the VH CDR2 region comprises an amino acid sequence that is at least 80% identical to a selected VH CDR2 amino acid sequence, and the VH CDR3 region comprises an amino acid sequence that is at least 80% identical to a selected VH CDR3 amino acid sequence. In some embodiments, the VL CDR1 region comprises an amino acid sequence that is at least 80% identical to a selected VL CDR1 amino acid sequence, the VL CDR2 region comprises an amino acid sequence that is at least 80% identical to a selected VL CDR2 amino acid sequence, and the VL CDR3 region comprises an amino acid sequence that is at least 80% identical to a selected VL CDR3 amino acid sequence. In some embodiments, the selected VH CDRs 1, 2, and 3 amino acid sequences and the selected VL CDRs, 1, 2, and 3 amino acid sequences are one of the following:
(1) the selected VH CDRs 1, 2, 3 amino acid sequences are set forth in SEQ ID NOs: 25, 26, and 27, respectively, and the selected VL CDRs 1, 2, 3 amino acid sequences are set forth in SEQ ID NOs: 28, 29, and 30, respectively;
(2) the selected VH CDRs 1, 2, 3 amino acid sequences are set forth in SEQ ID NOs: 25, 26, and 27, respectively, and the selected VL CDRs 1, 2, 3 amino acid sequences are set forth in SEQ ID NOs: 31, 32, and 33, respectively;
(3) the selected VH CDRs 1, 2, 3 amino acid sequences are set forth in SEQ ID NOs: 25, 26, and 27, respectively, and the selected VL CDRs 1, 2, 3 amino acid sequences are set forth in SEQ ID NOs: 34, 35, and 36, respectively;
(4) the selected VH CDRs 1, 2, 3 amino acid sequences are set forth in SEQ ID NOs: 25, 26, and 27, respectively, and the selected VL CDRs 1, 2, 3 amino acid sequences are set forth in SEQ ID NOs: 37, 38, and 39, respectively;
(5) the selected VH CDRs 1, 2, 3 amino acid sequences are set forth in SEQ ID NOs: 25, 26, and 27, respectively, and the selected VL CDRs 1, 2, 3 amino acid sequences are set forth in SEQ ID NOs: 40, 41, and 42, respectively; and
(6) the selected VH CDRs 1, 2, 3 amino acid sequences are set forth in SEQ ID NOs: 25, 26, and 27, respectively, and the selected VL CDRs 1, 2, 3 amino acid sequences are set forth in SEQ ID NOs: 43, 44, and 45, respectively.

In some embodiments, the heavy chain variable region (VH) comprises an amino acid sequence that is at least 90% identical to SEQ ID NO: 54, and a light chain variable region (VL) comprising an amino acid sequence that is at least 90% identical to SEQ ID NO: 55, 56, 57, 58, 59, or 60.

In some embodiments, the second antigen-binding site specifically binds to BCMA, and the second antigen-binding site comprises a heavy-chain antibody variable domain (VHH) comprising complementarity determining regions (CDRs) 1, 2, and 3. In some embodiments, the VHH CDR1 region comprises an amino acid sequence that is at least 80% identical to a selected VHH CDR1 amino acid sequence, the VHH CDR2 region comprises an amino acid sequence that is at least 80% identical to a selected VHH CDR2 amino acid sequence, and the VHH CDR3 region comprises an amino acid sequence that is at least 80% identical to a selected VHH CDR3 amino acid sequence. In some embodiments, the selected VHH CDRs 1, 2, and 3 amino acid sequences are one of the following:
(1) the selected VHH CDRs 1, 2, 3 amino acid sequences are set forth in SEQ ID NOs: 1, 2, and 3, respectively;
(2) the selected VHH CDRs 1, 2, 3 amino acid sequences are set forth in SEQ ID NOs: 4, 5, and 6, respectively;
(3) the selected VHH CDRs 1, 2, 3 amino acid sequences are set forth in SEQ ID NOs: 7, 8, and 9, respectively; and
(4) the selected VHH CDRs 1, 2, 3 amino acid sequences are set forth in SEQ ID NOs: 10, 11, and 12, respectively.

In some embodiments, the heavy-chain antibody variable domain (VHH) comprises an amino acid sequence that is at least 80% identical to a selected VHH sequence. In some embodiments, the selected VHH sequence is selected from the group consisting of SEQ ID NOs: 46-49.

In some embodiments, the second antigen-binding site specifically binds to CD38. In some embodiments, the second antigen-binding site comprises a heavy-chain antibody variable domain (VHH) comprising complementarity determining regions (CDRs) 1, 2, and 3. In some embodiments, the VHH CDR1 region comprises an amino acid sequence that is at least 80% identical to SEQ ID NO: 13, the VHH CDR2 region comprises an amino acid sequence that is at least 80% identical to SEQ ID NO: 14, and the VHH CDR3 region comprises an amino acid sequence that is at least 80% identical to SEQ ID NO: 15.

In some embodiments, the heavy-chain antibody variable domain (VHH) comprises an amino acid sequence that is at least 80% identical to SEQ ID NO: 50.

In some embodiments, the VH and the VL are linked by a linker peptide sequence to form an scFv.

In some embodiments, the linker peptide sequence comprises a sequence that is at least 80% identical to any one of SEQ ID NOs: 71-73 and 124-126.

In one aspect, the disclosure is related to a polypeptide complex, comprising (a) a first polypeptide comprising from N-terminus to C-terminus: a first heavy-chain antibody variable domain (VHH), a first hinge region, a first Fc region; and (b) a second polypeptide comprising from N-terminus to C-terminus: a second VHH, a second hinge region, and a second Fc region. In some embodiments, the first VHH specifically binds to BCMA or CD38. In some embodiments, the second VHH specifically binds to CD3.

In some embodiments, the first hinge region and/or the second hinge region comprise a sequence that is at least 80% identical to any one of SEQ ID NOs: 61-65.

In some embodiments, the first Fc region and/or the second Fc region comprise a sequence that is at least 80% identical to SEQ ID NO: 66 or 67.

In some embodiments, the first polypeptide comprises a sequence that is at least 80% identical to SEQ ID NO: 74, 75, 116, or 117. In some embodiments, the second polypeptide comprises a sequence that is at least 80% identical to SEQ ID NOs: 76, 77, 118, or 119.

In some embodiments, the first polypeptide further comprises a third VHH that specifically binds to BCMA or CD38. In some embodiments, the third VHH is linked to the N-terminus of the first VHH via a linker peptide sequence.

In some embodiments, the linker peptide sequence comprises a sequence that is at least 80% identical to any one of SEQ ID NOs: 71-73 and 124-126.

In some embodiments, the first polypeptide comprises a sequence that is at least 80% identical to SEQ ID NO: 78 or 79. In some embodiments, the second polypeptide comprises a sequence that is at least 80% identical to SEQ ID NO: 80 or 81.

In one aspect, the disclosure is related to a polypeptide complex, comprising (a) a first polypeptide comprising from N-terminus to C-terminus: a first heavy-chain antibody variable domain (VHH), a first hinge region, a first Fc region; (b) a second polypeptide comprising from N-terminus to C-terminus: a heavy chain variable region (VH), a second hinge region, and a second Fc region; and (c) a third polypeptide comprising a light chain variable region (VL). In some embodiments, the first VHH specifically binds to BCMA or CD38. In some embodiments, the VH and the VL associate with each other and specifically bind to CD3.

In some embodiments, the first hinge region and/or the second hinge region comprise a sequence that is at least 80% identical to any one of SEQ ID NOs: 61-65.

In some embodiments, the first Fc region and/or the second Fc region comprise a sequence that is at least 80% identical to SEQ ID NO: 66 or 67.

In some embodiments, the first polypeptide comprises a sequence that is at least 80% identical to SEQ ID NO: 82 or 83. In some embodiments, the second polypeptide comprises a sequence that is at least 80% identical to SEQ ID NO: 84 or 85. In some embodiments, the third polypeptide comprises a sequence that is at least 80% identical to SEQ ID NO: 86.

In some embodiments, the first polypeptide further comprises a second VHH that specifically binds to BCMA or CD38. In some embodiments, the second VHH is linked to the N-terminus of the first VHH via a linker peptide sequence.

In some embodiments, the linker peptide sequence comprises a sequence that is at least 80% identical to any one of SEQ ID NOs: 71-73 and 124-126.

In some embodiments, the first polypeptide comprises a sequence that is at least 80% identical to SEQ ID NO: 87 or 88. In some embodiments, the second polypeptide comprises a sequence that is at least 80% identical to SEQ ID NO: 89 or 90. In some embodiments, the third polypeptide comprises a sequence that is at least 80% identical to SEQ ID NO: 91.

In one aspect, the disclosure is related to a polypeptide complex, comprising (a) a first polypeptide comprising from N-terminus to C-terminus: a first heavy-chain antibody variable domain (VHH), a first hinge region, a first Fc region; and (b) a second polypeptide comprising from N-terminus to C-terminus: a single-chain variable fragment (scFv), a second hinge region, and a second Fc region. In some embodiments, the first VHH specifically binds to BCMA or CD38. In some embodiments, the scFv comprises a heavy chain variable region (VH), a first linker peptide sequence, and a light chain variable region (VL). In some embodiments, the VH and the VL associate with each other and specifically bind to CD3.

In some embodiments, the first linker peptide sequence comprises a sequence that is at least 80% identical to any one of SEQ ID NOs: 71-73 and 124-126.

In some embodiments, the first hinge region and/or the second hinge region comprise a sequence that is at least 80% identical to any one of SEQ ID NOs: 61-65.

In some embodiments, the first Fc region and/or the second Fc region comprise a sequence that is at least 80% identical to SEQ ID NO: 66 or 67.

In some embodiments, the first polypeptide comprises a sequence that is at least 80% identical to SEQ ID NO: 92, 93, 120, or 121. In some embodiments, the second polypeptide comprises a sequence that is at least 80% identical to SEQ ID NO: 94, 95, 122, or 123.

In some embodiments, the first polypeptide further comprises a second VHH that specifically binds to BCMA or CD38. In some embodiments, the second VHH is linked to the N-terminus of the first VHH via a second linker peptide sequence.

In some embodiments, the second linker peptide sequence comprises a sequence that is at least 80% identical to any one of SEQ ID NOs: 71-73 and 124-126.

In some embodiments, the first polypeptide comprises a sequence that is at least 80% identical to SEQ ID NO: 96 or 97. In some embodiments, the second polypeptide comprises a sequence that is at least 80% identical to SEQ ID NO: 98 or 99.

In one aspect, the disclosure is related to a polypeptide complex, comprising (a) a first polypeptide comprising from N-terminus to C-terminus: a first heavy chain variable region (VH1), a first hinge region, a first Fc region; a first linker peptide sequence, and a first heavy-chain antibody variable domain (VHH1); (b) a second polypeptide comprising a first light chain variable region (VL1); (c) a third polypeptide comprising from N-terminus to C-terminus: a second heavy chain variable region (VH2), a second hinge region, a second Fc region, a second linker peptide sequence, and a second heavy-chain antibody variable domain (VHH2); and (d) a fourth polypeptide comprising a second light chain variable region (VL2). In some embodiments, the VHH1 and/or the VHH2 specifically bind to BCMA or CD38. In some embodiments, the VH1 and the VL1 associate with each other and specifically bind to CD3. In some embodiments, the VH2 and the VL2 associate with each other and specifically bind to CD3.

In some embodiments, the first linker peptide sequence and/or the second linker peptide sequence comprises a sequence that is at least 80% identical to any one of SEQ ID NOs: 71-73 and 124-126.

In some embodiments, the first hinge region and/or the second hinge regions comprise a sequence that is at least 80% identical to any one of SEQ ID NOs: 61-65.

In some embodiments, sequences of the VHH1 and the VHH2 are identical. In some embodiments, the first Fc region and/or the second Fc region comprise a sequence that is at least 80% identical to SEQ ID NO: 68.

In some embodiments, the first polypeptide comprises a sequence that is at least 80% identical to SEQ ID NO: 100 or 101. In some embodiments, the second polypeptide comprises a sequence that is at least 80% identical to SEQ ID NO: 102. In some embodiments, the third polypeptide comprises a sequence that is at least 80% identical to SEQ ID NO: 100 or 101. In some embodiments, the fourth polypeptide comprises a sequence that is at least 80% identical to SEQ ID NO: 102.

In some embodiments, sequences of the VHH1 and the VHH2 are different. In some embodiments, the first Fc region and/or the second Fc region comprise a sequence that is at least 80% identical to SEQ ID NO: 66 or 67.

In some embodiments, the first polypeptide comprises a sequence that is at least 80% identical to SEQ ID NO: 103 or 104. In some embodiments, the second polypeptide comprises a sequence that is at least 80% identical to SEQ ID NO: 107. In some embodiments, the third polypeptide comprises a sequence that is at least 80% identical to SEQ ID NO: 105 or 106. In some embodiments, the fourth polypeptide comprises a sequence that is at least 80% identical to SEQ ID NO: 107.

In one aspect, the disclosure is related to a polypeptide complex, comprising (a) a first polypeptide comprising from N-terminus to C-terminus: a first heavy-chain antibody variable domain (VHH1), a first linker peptide sequence, a first heavy chain variable region (VH1), a first hinge region, and a first Fc region; (b) a second polypeptide comprising a first light chain variable region (VL1); (c) a third polypeptide comprising from N-terminus to C-terminus: a second heavy-chain antibody variable domain (VHH2), a second linker peptide sequence, a second heavy chain variable region (VH2), a second hinge region, and a second Fc region; and (d) a fourth polypeptide comprising a second light chain variable region (VL2). In some embodiments, the VHH1 and the VHH2 specifically bind to BCMA or CD38. In some embodiments, the VH1 and the VL1 associate with each other and specifically bind to CD3. In some embodiments, the VH2 and the VL2 associate with each other and specifically bind to CD3.

In some embodiments, the first linker peptide sequence and/or the second linker peptide sequence comprises a sequence that is at least 80% identical to any one of SEQ ID NOs: 71-73 and 124-126.

In some embodiments, the first hinge region and/or the second hinge regions comprise a sequence that is at least 80% identical to any one of SEQ ID NOs: 61-65.

In some embodiments, sequences of the VHH1 and the VHH2 are identical. In some embodiments, the first Fc region and/or the second Fc region comprise a sequence that is at least 80% identical to SEQ ID NO: 68.

In some embodiments, the first polypeptide comprises a sequence that is at least 80% identical to SEQ ID NO: 108 or 109. In some embodiments, the second polypeptide comprises a sequence that is at least 80% identical to SEQ ID NO: 110. In some embodiments, the third polypeptide comprises a sequence that is at least 80% identical to SEQ ID NO: 108 or 109. In some embodiments, the fourth polypeptide comprises a sequence that is at least 80% identical to SEQ ID NO: 110.

In some embodiments, sequences of the VHH1 and the VHH2 are different. In some embodiments, the first Fc region and/or the second Fc region comprise a sequence that is at least 80% identical to SEQ ID NO: 66 or 67.

In some embodiments, the first polypeptide comprises a sequence that is at least 80% identical to SEQ ID NO: 111 or 112. In some embodiments, the second polypeptide comprises a sequence that is at least 80% identical to SEQ ID NO: 115. In some embodiments, the third polypeptide comprises a sequence that is at least 80% identical to SEQ ID NO: 113 or 114. In some embodiments, the fourth polypeptide comprises a sequence that is at least 80% identical to SEQ ID NO: 115.

In one aspect, the disclosure is related to a nucleic acid comprising a polynucleotide encoding the antibody or antigen-binding fragment thereof as described herein, the multi-specific antibody or antigen-binding fragment thereof as described herein, or the polypeptide complex as describe herein.

In some embodiments, the nucleic acid is a DNA (e.g., cDNA) or RNA (e.g., mRNA).

In one aspect, the disclosure is related to a vector comprising one or more of the nucleic acids as described herein.

In one aspect, the disclosure is related to a cell comprising the vector as described herein. In some embodiments, the cell is a CHO cell.

In one aspect, the disclosure is related to a cell comprising one or more of the nucleic acids as described herein.

In one aspect, the disclosure is related to a method of producing an antibody or an antigen-binding fragment thereof, the method comprising (a) culturing the cell as described herein under conditions sufficient for the cell to produce the antibody or the antigen-binding fragment; and (b) collecting the antibody or the antigen-binding fragment produced by the cell.

In one aspect, the disclosure is related to an antibody-drug conjugate comprising the antibody or antigen-binding fragment thereof as described herein, or the multi-specific antibody or antigen-binding fragment thereof as described herein, covalently bound to a therapeutic agent.

In some embodiments, the therapeutic agent is a cytotoxic or cytostatic agent.

In one aspect, the disclosure is related to a method of treating a subject having cancer, the method comprising administering a therapeutically effective amount of a composition comprising the antibody or antigen-binding fragment thereof as described herein, the multi-specific antibody or antigen-binding fragment thereof as described herein, the polypeptide complex as described herein, or the antibody-drug conjugate as described herein, to the subject.

In some embodiments, the subject has a cancer expressing BCMA or CD38.

In some embodiments, the cancer is multiple myeloma. In some embodiments, the cancer is hematological malignancy.

In one aspect, the disclosure is related to a method of decreasing the rate of tumor growth, the method comprising contacting a tumor cell with an effective amount of a composition comprising the antibody or antigen-binding fragment thereof described herein, the multi-specific antibody or antigen-binding fragment thereof as described herein, the polypeptide complex as described herein, or the antibody-drug conjugate as described herein.

In one aspect, the disclosure is related to a method of killing a tumor cell, the method comprising contacting a tumor cell with an effective amount of a composition comprising the antibody or antigen-binding fragment thereof as described herein, the multi-specific antibody or antigen-binding fragment thereof as described herein, the polypeptide complex as described herein, or the antibody-drug conjugate as described herein.

In one aspect, the disclosure is related to a method of killing a tumor cell, the method comprising contacting a tumor cell with an effective amount of a composition comprising the antibody or antigen-binding fragment thereof as described herein, the multi-specific antibody or antigen-binding fragment thereof as described herein, the polypeptide complex as described herein, or the antibody-drug conjugate as described herein.

In one aspect, the disclosure is related to a pharmaceutical composition comprising the antibody or antigen-binding fragment thereof as described herein, the multi-specific antibody or antigen-binding fragment thereof as described herein, or the polypeptide complex as described herein, and a pharmaceutically acceptable carrier.

In one aspect, the disclosure is related to a pharmaceutical composition comprising the antibody-drug conjugate as described herein, and a pharmaceutically acceptable carrier.

In one aspect, the disclosure provides a multi-specific antibody or antigen-binding fragment thereof, comprising the heavy-chain antibody variable domain (VHH) as described herein, or the heavy chain variable region (VH) and the light chain variable region (VL) as described herein.

In one aspect, the disclosure provides a bispecific antibody or antigen-binding fragment thereof, comprising the heavy-chain antibody variable domain (VHH) as described herein, or the heavy chain variable region (VH) and the light chain variable region (VL) as described herein.

In one aspect, the disclosure provides a multi-specific antibody or antigen-binding fragment thereof that specifically binds to BCMA and CD38.

As used herein, the term “antibody” refers to any antigen-binding molecule that contains at least one (e.g., one, two, three, four, five, or six) complementary determining region (CDR) (e.g., any of the three CDRs from an immunoglobulin light chain or any of the three CDRs from an immunoglobulin heavy chain) and is capable of specifically binding to an epitope in an antigen. Non-limiting examples of antibodies include: monoclonal antibodies, polyclonal antibodies, multi-specific antibodies (e.g., bi-specific antibodies), single-chain antibodies, single variable domain (VHH) antibodies, chimeric antibodies, human antibodies, and humanized antibodies. In some embodiments, an antibody can contain an Fc region of a human antibody. The term antibody also includes derivatives, e.g., multi-specific antibodies, bi-specific antibodies, single-chain antibodies, diabodies, and linear antibodies formed from these antibodies or antibody fragments.

As used herein, the term “antigen-binding fragment” refers to a portion of a full-length antibody, wherein the portion of the antibody is capable of specifically binding to an antigen. In some embodiments, the antigen-binding fragment contains at least one variable domain (e.g., a variable domain of a heavy chain, a variable domain of light chain or a VHH). Non-limiting examples of antibody fragments include, e.g., Fab, Fab′, F(ab′)2, and Fv fragments, ScFv, and VHH.

As used herein, the terms “subject” and “patient” are used interchangeably throughout the specification and describe an animal, human or non-human, to whom treatment according to the methods of the present invention is provided. Veterinary and non-veterinary applications are contemplated in the present disclosure. Human patients can be adult humans or juvenile humans (e.g., humans below the age of 18 years old). In addition to humans, patients include but are not limited to mice, rats, hamsters, guinea-pigs, rabbits, ferrets, cats, dogs, and primates. Included are, for example, non-human primates (e.g., monkey, chimpanzee, gorilla, and the like), rodents (e.g., rats, mice, gerbils, hamsters, ferrets, rabbits), lagomorphs, swine (e.g., pig, miniature pig), equine, canine, feline, bovine, and other domestic, farm, and zoo animals.

As used herein, when referring to an antibody or an antigen-binding fragment, the phrases “specifically binding” and “specifically binds” mean that the antibody or an antigen-binding fragment interacts with its target molecule preferably to other molecules, because the interaction is dependent upon the presence of a particular structure (i.e., the antigenic determinant or epitope) on the target molecule; in other words, the reagent is recognizing and binding to molecules that include a specific structure rather than to all molecules in general. An antibody that specifically binds to the target molecule may be referred to as a target-specific antibody. For example, an antibody that specifically binds to CD3 may be referred to as a CD3-specific antibody or an anti-CD3 antibody.

As used herein, the term “bispecific antibody” refers to an antibody that binds to two different epitopes. The epitopes can be on the same antigen or on different antigens.

As used herein, the term “trispecific antibody” refers to an antibody that binds to three different epitopes. The epitopes can be on the same antigen or on different antigens.

As used herein, the term “multispecific antibody” refers to an antibody that binds to two or more different epitopes. The epitopes can be on the same antigen or on different antigens. A multispecific antibody can be e.g., a bispecific antibody or a trispecific antibody. In some embodiments, the multispecific antibody binds to two, three, four, five, or six different epitopes.

As used herein, a “VHH” refers to the variable domain of a heavy chain antibody. In some embodiments, the VHH is a humanized VHH.

As used herein, the terms “polypeptide,” “peptide,” and “protein” are used interchangeably to refer to polymers of amino acids of any length of at least two amino acids.

As used herein, the terms “polynucleotide,” “nucleic acid molecule,” and “nucleic acid sequence” are used interchangeably herein to refer to polymers of nucleotides of any length of at least two nucleotides, and include, without limitation, DNA, RNA, DNA/RNA hybrids, and modifications thereof.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1 shows specific binding of BCMA/CD3 antibodies to the target cells. Bispecific antibodies 3B2/CD3, 3D7/CD3, 3E1/CD3, 3E5/CD3 with the BiSpecific-V1 structure or trispecific antibody 3E1-3B2/CD3 with the TriSpecific-V1 structure were incubated with Raji and Jurkat cells, respective. “Cell Only” and secondary antibody only (“2° Ab only”) are negative controls.

FIG. 2 shows BCMA/CD3 antibody-mediated Jurkat cell activation. Jurkat and Raji cells were co-incubated in the presence of 10 μg/ml of BCMA/CD3 bispecific antibodies 3B2/CD3, 3D7/CD3, 3E1/CD3, 3E5/CD3 with the BiSpecific-V1 structure or trispecific antibody 3E1-3B2/CD3 with the TriSpecific-V1 structure. The ratio of Raji cells to Jurkat cells was 1:1. After overnight incubation, the cells were stained with PE-conjugated anti-CD69 antibody and APC-conjugated anti-CD2 antibody, and the cell samples were analyzed by FACS. “Isotype Ab” is an isotype control.

FIG. 3 shows CD38/CD3 antibody-mediated Jurkat cell activation. Jurkat and CD38-expressing 293 cells were co-incubated in the presence of 8 μg/ml of CD38-F2/CD3 bispecific antibodies CD38-F2/BsAb-v0 (with the BiSpecific-V0 structure) or CD38-F2-BsAb-v2 (with the BiSpecific-V2 structure). The ratio of CD38-expressing 293 cells to Jurkat cells was 1:1. After overnight incubation, the cells were stained with PE-conjugated anti-CD69 antibody, and the cell samples were analyzed by FACS.

FIG. 4A shows a schematic structure of an exemplary BCMA/CD3 bispecific antibody with the BiSpecific-V0 format.

FIG. 4B shows a schematic structure of an exemplary BCMA/CD3 trispecific antibody with the TriSpecific-V0 format.

FIG. 5A shows a schematic structure of an exemplary BCMA/CD3 bispecific antibody with the BiSpecific-V1 format.

FIG. 5B shows a schematic structure of an exemplary BCMA/CD3 trispecific antibody with the TriSpecific-V1 format.

FIG. 6A shows a schematic structure of an exemplary BCMA/CD3 bispecific antibody with the BiSpecific-V2 format.

FIG. 6B shows a schematic structure of an exemplary BCMA/CD3 trispecific antibody with the TriSpecific-V2 format.

FIG. 7A shows a schematic structure of an exemplary BCMA/CD3 bispecific antibody with the BiSpecific-V3 format.

FIG. 7B shows a schematic structure of an exemplary BCMA/CD3 trispecific antibody with the TriSpecific-V3 format.

FIG. 8A shows a schematic structure of an exemplary BCMA/CD3 bispecific antibody with the BiSpecific-V4 format.

FIG. 8B shows a schematic structure of an exemplary BCMA/CD3 trispecific antibody with the TriSpecific-V4 format.

FIG. 9A shows a schematic structure of an exemplary CD38/CD3 bispecific antibody with the BiSpecific-V0 format.

FIG. 9B shows a schematic structure of an exemplary CD38/CD3 trispecific antibody with the TriSpecific-V0 format.

FIG. 10A shows a schematic structure of an exemplary CD38/CD3 bispecific antibody with the BiSpecific-V1 format.

FIG. 10B shows a schematic structure of an exemplary CD38/CD3 trispecific antibody with the TriSpecific-V1 format.

FIG. 11A shows a schematic structure of an exemplary CD38/CD3 bispecific antibody with the BiSpecific-V2 format.

FIG. 11B shows a schematic structure of an exemplary CD38/CD3 trispecific antibody with the TriSpecific-V2 format.

FIG. 12A shows a schematic structure of an exemplary CD38/CD3 bispecific antibody with the BiSpecific-V3 format.

FIG. 12B shows a schematic structure of an exemplary CD38/CD3 trispecific antibody with the TriSpecific-V3 format.

FIG. 13A shows a schematic structure of an exemplary CD38/CD3 bispecific antibody with the BiSpecific-V4 format.

FIG. 13B shows a schematic structure of an exemplary CD38/CD3 trispecific antibody with the TriSpecific-V4 format.

FIG. 14 shows a schematic structure of an exemplary trispecific antibody with the TriSpecific-V0 format.

FIG. 15 shows a schematic structure of an exemplary trispecific antibody with the TriSpecific-V1 format.

FIG. 16 shows a schematic structure of an exemplary trispecific antibody with the TriSpecific-V2 format.

FIG. 17A shows a schematic structure of an exemplary trispecific antibody with the TriSpecific-V3 format.

FIG. 17B shows a schematic structure of an exemplary trispecific antibody with the TriSpecific-V4 format.

FIG. 18A shows a schematic structure of an exemplary bispecific antibody with the BiSpecific-V0 format.

FIG. 18B shows a schematic structure of an exemplary trispecific antibody with the TriSpecific-V0 format.

FIG. 19 is a schematic diagram showing how a bispecific antibody that binds to CD3 and a cancer antigen (e.g., cancer specific antigen) can recognize and kill a tumor cell.

FIG. 20A shows CD38 and BCMA expression levels in HCl-H929 (H929) and Raji cells.

FIGS. 20B-20E show the antibody binding curves to HCl-H929, Jurkat, Raji, and 293 cells, respectively. Benchmark CD38/CD3 and BCMA/CD3 bispecific antibodies were used as positive controls. “COVID” or “Negative control” is a non-specific antibody control.

FIG. 21A shows the activation curves of Jurkat cells when mixed with Raji cells and treated with CD38/CD3 bispecific or CD38/BCMA/CD3 trispecific antibodies.

FIG. 21B shows the activation curves of Jurkat cells when mixed with CHO cells and treated with CD38/CD3 bispecific or CD38/BCMA/CD3 trispecific antibodies.

FIG. 21C shows the activation curves of Jurkat cells when mixed with HCl-H929 cells and treated with CD38/CD3, BCMA/CD3 bispecific or CD38/BCMA/CD3 trispecific antibodies. “COVID” is a non-specific antibody control.

FIG. 21D shows the activation curves of Jurkat cells when mixed with 293 cells and treated with CD38/CD3, BCMA/CD3 bispecific or CD38/BCMA/CD3 trispecific antibodies. “COVID” is a non-specific antibody control.

FIG. 22A shows the killing curves of HCl-929 cells when mixed with PBMCs and treated with CD38/CD3, BCMA/CD3 bispecific or CD38/BCMA/CD3 trispecific antibodies. Benchmark CD38/CD3 and BCMA/CD3 bispecific antibodies were used as positive controls.

FIG. 22B shows the T cell activation curves when PBMCs were mixed with HCl-929 cells and treated with CD38/CD3, BCMA/CD3 bispecific or CD38/BCMA/CD3 trispecific antibodies. Benchmark CD38/CD3 and BCMA/CD3 bispecific antibodies were used as positive controls.

FIG. 23 lists CDR sequences of the VHHs from the BCMA, CD38, or CD3 antibodies described in the disclosure.

FIG. 24 lists CDR sequences of the heavy chain variable region (VH) of the CD3-12F6 antibody.

FIG. 25 lists CDR sequences of the light chain variable regions (VLs) of the CD3 antibodies described in the disclosure.

FIG. 26 lists amino acid sequences of VHHs that specifically bind to BCMA, CD38, or CD3 as described in the disclosure.

FIG. 27 lists amino acid sequences of VH and VLs that specifically bind to CD3 as described in the disclosure.

FIG. 28 lists sequences discussed in the disclosure.

### DETAILED DESCRIPTION

A multi-specific antibody (e.g., bispecific antibody or a trispecific antibody) or antigen-binding fragment thereof is an artificial protein that can simultaneously bind to two or more different types of epitopes. The epitopes can be in the same antigen or in different antigens. In some embodiments, a multi-specific antibody or antigen-binding fragment thereof can have two, three, four, five, six, or more antigen binding sites. In some embodiments, the antigen binding site has one heavy chain variable region and one light chain variable region. In some embodiments, the antigen binding site has one VHH.

The present disclosure provides multispecific antibodies or antigen-binding fragments thereof that binds to CD3 and one or more cancer antigens (e.g., BCMA, and/or CD38, or a combination thereof).

CD3 (cluster of differentiation 3) is a protein complex and T cell co-receptor that is involved in activating both the cytotoxic T cell (CD8+ naive T cells) and T helper cells (CD4+ naive T cells). It is composed of four distinct chains. In mammals, the complex contains a CD37 chain, a CD3δ chain, and two CD3ϑ chains. These chains associate with the T-cell receptor (TCR) and the ζ-chain (zeta-chain) to generate an activation signal in T lymphocytes. The TCR, ζ-chain, and CD3 molecules together constitute the TCR complex.

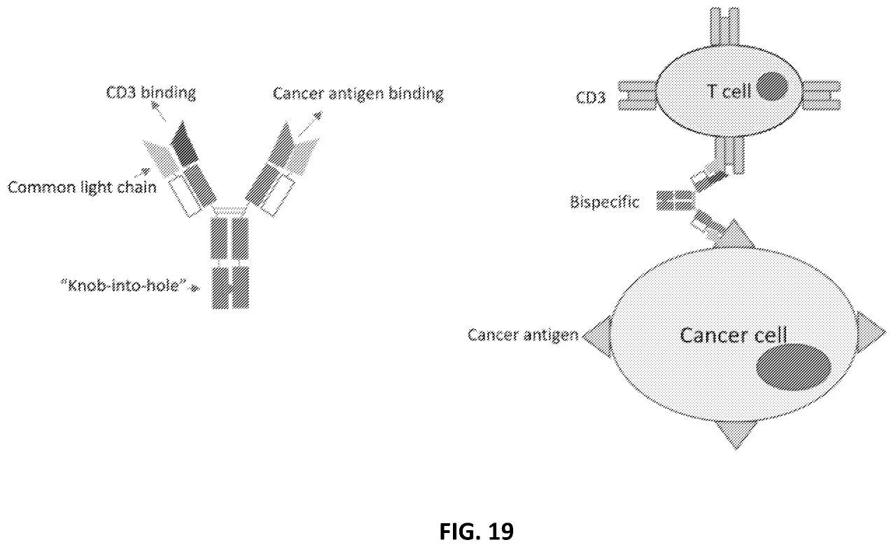

Because CD3 is involved in T cell activation, monoclonal antibodies that target it are being investigated as therapies for cancers. However, the anti-CD3 antibody by itself may activate T cells, causing uncontrolled immune response. Thus, the binding affinity with CD3 should be carefully adjusted. In many cases, the binding affinity to the cancer antigen is greater than the binding affinity to CD3. These multispecific antibodies with imbalanced affinities can have various advantages. For example, multispecific antibodies with imbalanced affinities can be used to target a cancer antigen on cancer cells and CD3 on T cell. In this case, high affinity to the cancer antigen can lead to better capturing of cancer cells by T cells, and low affinity to CD3 can avoid triggering T-cell signaling by CD3 in the absence of the cancer antigen (FIG. 19). Only when the multispecific antibodies is presented to the T cell in a multivalent fashion by a target cancer cell, can the multispecific antibodies be activated and kill the target cancer cell.

The present disclosure provides multispecific antibodies or antigen-binding fragments thereof that binds to CD3 and cancer antigens. The binding affinity to CD3 is carefully adjusted. The present disclosure further shows that the multispecific antibodies or antigen-binding fragments as described herein can only activate T cells in the presence of cells that express cancer antigens. This can greatly improve the safety and the efficacy of the multispecific antibodies. The present disclosure further provides anti-BCMA, anti-CD3, anti-CD38 VHH and antibodies having a VH and a VL or antigen binding fragments thereof. These VHH, VH, VL can be used to make various multispecific antibodies or antigen-binding fragments as described herein.

### Heavy-Chain Antibody Variable Domain (VHH)

Monoclonal and recombinant antibodies are important tools in medicine and biotechnology. Like all mammals, camelids (e.g., llamas) can produce conventional antibodies made of two heavy chains and two light chains bound together with disulfide bonds in a Y shape (e.g., IgG1). However, they also produce two unique subclasses of IgG: IgG2 and IgG3, also known as heavy chain antibody. These antibodies are made of only two heavy chains, which lack the CH1 region but still bear an antigen-binding domain at their N-terminus called VHH (or nanobody). Conventional Ig require the association of variable regions from both heavy and light chains to allow a high diversity of antigen-antibody interactions. Although isolated heavy and light chains still show this capacity, they exhibit very low affinity when compared to paired heavy and light chains. The unique feature of heavy chain antibody is the capacity of their monomeric antigen binding regions to bind antigens with specificity, affinity and especially diversity that are comparable to conventional antibodies without the need of pairing with another region. This feature is mainly due to a couple of major variations within the amino acid sequence of the variable region of the two heavy chains, which induce deep conformational changes when compared to conventional Ig. Major substitutions in the variable regions prevent the light chains from binding to the heavy chains, but also prevent unbound heavy chains from being recycled by the Immunoglobulin Binding Protein.

The single variable domain of these antibodies (designated VHH, sdAb, nanobody, or heavy-chain antibody variable domain) is the smallest antigen-binding domain generated by adaptive immune systems. The third Complementarity Determining Region (CDR3) of the variable region of these antibodies has often been found to be twice as long as the conventional ones. This results in an increased interaction surface with the antigen as well as an increased diversity of antigen-antibody interactions, which compensates the absence of the light chains. With a long complementarity-determining region 3 (CDR3), VHHs can extend into crevices on proteins that are not accessible to conventional antibodies, including functionally interesting sites such as the active site of an enzyme or the receptor-binding canyon on a virus surface. Moreover, an additional cysteine residue allow the structure to be more stable, thus increasing the strength of the interaction.

VHHs offer numerous other advantages compared to conventional antibodies carrying variable domains (VH and VL) of conventional antibodies, including higher stability, solubility, expression yields, and refolding capacity, as well as better in vivo tissue penetration. Moreover, in contrast to the VH domains of conventional antibodies VHH do not display an intrinsic tendency to bind to light chains. This facilitates the induction of heavy chain antibodies in the presence of a functional light chain loci. Further, since VHH do not bind to VL domains, it is much easier to reformat VHHs into multispecific antibody constructs than constructs containing conventional VH-VL pairs or single domains based on VH domains.

The disclosure provides e.g., anti-BCMA antibodies, the modified antibodies thereof, the chimeric antibodies thereof, and the humanized antibodies thereof. The disclosure also provides VHH of these antibodies. These VHHs can be used in various multispecific antibody constructs as described herein.

The CDR sequences for BCMA-3B2 (or 3B2), and BCMA-3B2 derived antibodies (e.g., humanized antibodies) include CDRs of the VHH domain as set forth in SEQ ID NOs: 1, 2, and 3, respectively. The amino acid sequence for the VHH domain of BCMA-3B2 antibody is set forth in SEQ ID NO: 46.

The CDR sequences for BCMA-3D7 (or 3D7), and BCMA-3D7 derived antibodies (e.g., humanized antibodies) include CDRs of the VHH domain as set forth in SEQ ID NOs: 4, 5, and 6, respectively. The amino acid sequence for the VHH domain of BCMA-3D7 antibody is set forth in SEQ ID NO: 47.

The CDR sequences for BCMA-3E1 (or 3E1), and BCMA-3E1 derived antibodies (e.g., humanized antibodies) include CDRs of the VHH domain as set forth in SEQ ID NOs: 7, 8, and 9, respectively. The amino acid sequence for the VHH domain of BCMA-3E1 antibody is set forth in SEQ ID NO: 48.

The CDR sequences for BCMA-3E5 (or 3E5), and BCMA-3E5 derived antibodies (e.g., humanized antibodies) include CDRs of the VHH domain as set forth in SEQ ID NOs: 10, 11, and 12, respectively. The amino acid sequence for the VHH domain of BCMA-3E5 antibody is set forth in SEQ ID NO: 49.

The disclosure provides e.g., anti-CD38 antibodies, the modified antibodies thereof, the chimeric antibodies thereof, and the humanized antibodies thereof. The CDR sequences for CD38-F2, and CD38-F2 derived antibodies (e.g., humanized antibodies) include CDRs of the VHH domain as set forth in SEQ ID NOs: 13, 14, and 15, respectively. The amino acid sequence for the VHH domain of CD38-F2 antibody is set forth in SEQ ID NO: 50.

The disclosure provides e.g., anti-CD3 antibodies, the modified antibodies thereof, the chimeric antibodies thereof, and the humanized antibodies thereof.

The CDR sequences for CD3-12F6, and CD3-12F6 derived antibodies (e.g., humanized antibodies) include CDRs of the VHH domain as set forth in SEQ ID NOs: 16, 17, and 18, respectively. The amino acid sequence for the VHH domain of CD38-F2 antibody is set forth in SEQ ID NO: 51.

The CDR sequences for CD3-OKT3-v1, and CD3-OKT3-v1 derived antibodies (e.g., humanized antibodies) include CDRs of the VHH domain as set forth in SEQ ID NOs: 19, 20, and 21, respectively. The amino acid sequence for the VHH domain of CD38-F2 antibody is set forth in SEQ ID NO: 52.

The CDR sequences for CD3-OKT3-v2, and CD3-OKT3-v2 derived antibodies (e.g., humanized antibodies) include CDRs of the VHH domain as set forth in SEQ ID NOs: 22, 23, and 24, respectively. The amino acid sequence for the VHH domain of CD38-F2 antibody is set forth in SEQ ID NO: 53.

The amino acid sequences for various modified or humanized VHH are also provided. As there are different ways to modify or humanize a llama antibody (e.g., a sequence can be modified with different amino acid substitutions), the heavy chain and the light chain of an antibody can have more than one version of humanized sequences. In some embodiments, the humanized VHH domain is at least 80%, 85%, 90%, 91%, 92%, 93%, 94%, 95%, 96%, 97%, 98%, or 99% identical to any sequence of SEQ ID NOs: 46-53.

Furthermore, in some embodiments, the antibodies or antigen-binding fragments thereof described herein can also contain one, two, or three VHH domain CDRs selected from the group of SEQ ID NOs: 1-3, SEQ ID NOs: 4-6, SEQ ID NOs: 7-9, SEQ ID NOs: 10-12, SEQ ID NOs: 13-15, SEQ ID NOs: 16-18, SEQ ID NOs: 19-21, and SEQ ID NOs: 22-24.

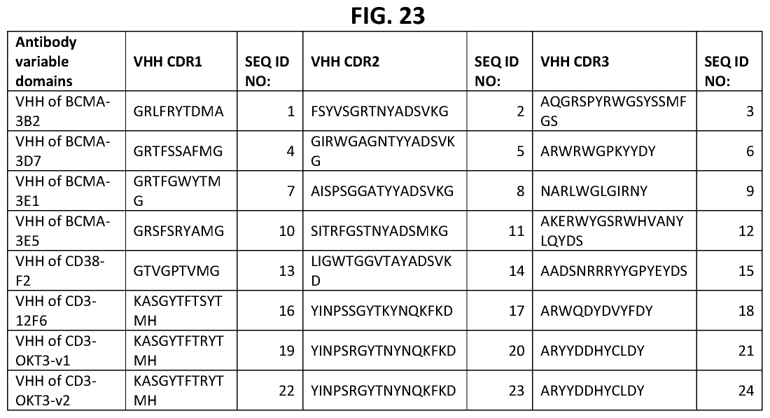

In some embodiments, the antibodies can have a heavy-chain antibody variable domain (VHH) comprising complementarity determining regions (CDRs) 1, 2, 3, wherein the CDR1 region comprises or consists of an amino acid sequence that is at least 80%, 85%, 90%, or 95% identical to a selected VHH CDR1 amino acid sequence, the CDR2 region comprises or consists of an amino acid sequence that is at least 80%, 85%, 90%, or 95% identical to a selected VHH CDR2 amino acid sequence, and the CDR3 region comprises or consists of an amino acid sequence that is at least 80%, 85%, 90%, or 95% identical to a selected VHH CDR3 amino acid sequence. The selected VHH CDRs 1, 2, 3 amino acid sequences is shown in FIG. 23.

In some embodiments, the antibody or an antigen-binding fragment described herein can contain a heavy-chain antibody variable domain (VHH) containing one, two, or three of VHH CDR1 with zero, one or two amino acid insertions, deletions, or substitutions; VHH CDR2 with zero, one or two amino acid insertions, deletions, or substitutions; VHH CDR3 with zero, one or two amino acid insertions, deletions, or substitutions, wherein VHH CDR1, VHH CDR2, and VHH CDR3 are selected from the CDRs in FIG. 23.

In some embodiments, the antibody or an antigen-binding fragment described herein can contain a heavy-chain antibody variable domain (VHH) containing one, two, or three of the CDRs of SEQ ID NO: 1 with zero, one or two amino acid insertions, deletions, or substitutions; SEQ ID NO: 2 with zero, one or two amino acid insertions, deletions, or substitutions; SEQ ID NO: 3 with zero, one or two amino acid insertions, deletions, or substitutions.

In some embodiments, the antibody or an antigen-binding fragment described herein can contain a heavy-chain antibody variable domain (VHH) containing one, two, or three of the CDRs of SEQ ID NO: 4 with zero, one or two amino acid insertions, deletions, or substitutions; SEQ ID NO: 5 with zero, one or two amino acid insertions, deletions, or substitutions; SEQ ID NO: 6 with zero, one or two amino acid insertions, deletions, or substitutions.

In some embodiments, the antibody or an antigen-binding fragment described herein can contain a heavy-chain antibody variable domain (VHH) containing one, two, or three of the CDRs of SEQ ID NO: 7 with zero, one or two amino acid insertions, deletions, or substitutions; SEQ ID NO: 8 with zero, one or two amino acid insertions, deletions, or substitutions; SEQ ID NO: 9 with zero, one or two amino acid insertions, deletions, or substitutions.

In some embodiments, the antibody or an antigen-binding fragment described herein can contain a heavy-chain antibody variable domain (VHH) containing one, two, or three of the CDRs of SEQ ID NO: 10 with zero, one or two amino acid insertions, deletions, or substitutions; SEQ ID NO: 11 with zero, one or two amino acid insertions, deletions, or substitutions; SEQ ID NO: 12 with zero, one or two amino acid insertions, deletions, or substitutions.

In some embodiments, the antibody or an antigen-binding fragment described herein can contain a heavy-chain antibody variable domain (VHH) containing one, two, or three of the CDRs of SEQ ID NO: 13 with zero, one or two amino acid insertions, deletions, or substitutions; SEQ ID NO: 14 with zero, one or two amino acid insertions, deletions, or substitutions; SEQ ID NO: 15 with zero, one or two amino acid insertions, deletions, or substitutions.

In some embodiments, the antibody or an antigen-binding fragment described herein can contain a heavy-chain antibody variable domain (VHH) containing one, two, or three of the CDRs of SEQ ID NO: 16 with zero, one or two amino acid insertions, deletions, or substitutions; SEQ ID NO: 17 with zero, one or two amino acid insertions, deletions, or substitutions; SEQ ID NO: 18 with zero, one or two amino acid insertions, deletions, or substitutions.

In some embodiments, the antibody or an antigen-binding fragment described herein can contain a heavy-chain antibody variable domain (VHH) containing one, two, or three of the CDRs of SEQ ID NO: 19 with zero, one or two amino acid insertions, deletions, or substitutions; SEQ ID NO: 20 with zero, one or two amino acid insertions, deletions, or substitutions; SEQ ID NO: 21 with zero, one or two amino acid insertions, deletions, or substitutions.

In some embodiments, the antibody or an antigen-binding fragment described herein can contain a heavy-chain antibody variable domain (VHH) containing one, two, or three of the CDRs of SEQ ID NO: 22 with zero, one or two amino acid insertions, deletions, or substitutions; SEQ ID NO: 23 with zero, one or two amino acid insertions, deletions, or substitutions; SEQ ID NO: 24 with zero, one or two amino acid insertions, deletions, or substitutions.

The insertions, deletions, and substitutions can be within the CDR sequence, or at one or both terminal ends of the CDR sequence. In some embodiments, the CDR is determined based on Kabat numbering scheme. In some embodiments, the CDR is determined based on Chothia numbering scheme. In some embodiments, the CDR is determined based on a combination numbering scheme.

The disclosure also provides antibodies or antigen-binding fragments thereof that bind to BCMA. The antibodies or antigen-binding fragments thereof contain a heavy-chain antibody variable domain (VHH) comprising or consisting of an amino acid sequence that is at least 80%, 85%, 90%, or 95% identical to a selected VHH sequence. In some embodiments, the selected VHH sequence is SEQ ID NO: 46. In some embodiments, the selected VHH sequence is SEQ ID NO: 47. In some embodiments, the selected VHH sequence is SEQ ID NO: 48. In some embodiments, the selected VHH sequence is SEQ ID NO: 49.

The disclosure also provides antibodies or antigen-binding fragments thereof that bind to CD38. The antibodies or antigen-binding fragments thereof contain a heavy-chain antibody variable domain (VHH) comprising or consisting of an amino acid sequence that is at least 80%, 85%, 90%, or 95% identical to SEQ ID NO: 50.

The disclosure also provides antibodies or antigen-binding fragments thereof that bind to CD3. The antibodies or antigen-binding fragments thereof contain a heavy-chain antibody variable domain (VHH) comprising or consisting of an amino acid sequence that is at least 80%, 85%, 90%, or 95% identical to a selected VHH sequence. In some embodiments, the selected VHH sequence is SEQ ID NO: 51. In some embodiments, the selected VHH sequence is SEQ ID NO: 52. In some embodiments, the selected VHH sequence is SEQ ID NO: 53.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences. For purposes of illustration, the comparison of sequences and determination of percent identity between two sequences can be accomplished, e.g., using a Blossum 62 scoring matrix with a gap penalty of 12, a gap extend penalty of 4, and a frameshift gap penalty of 5.

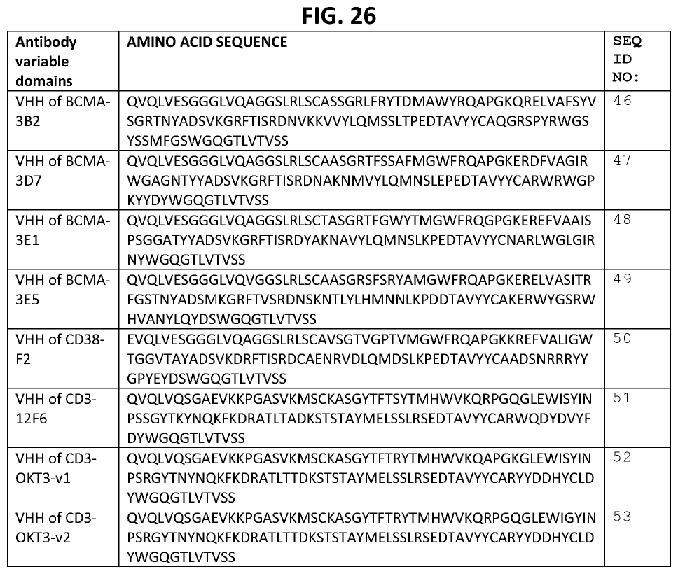

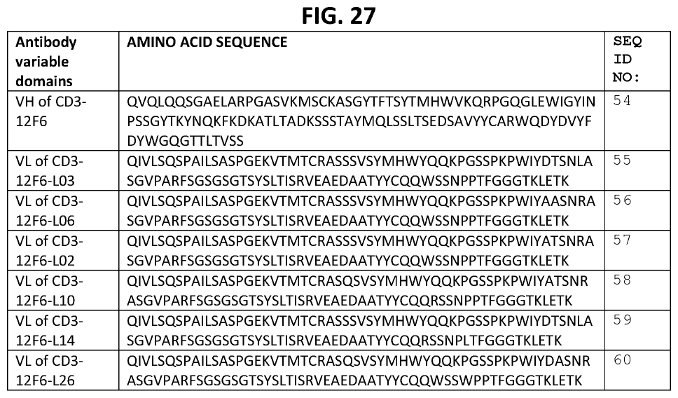

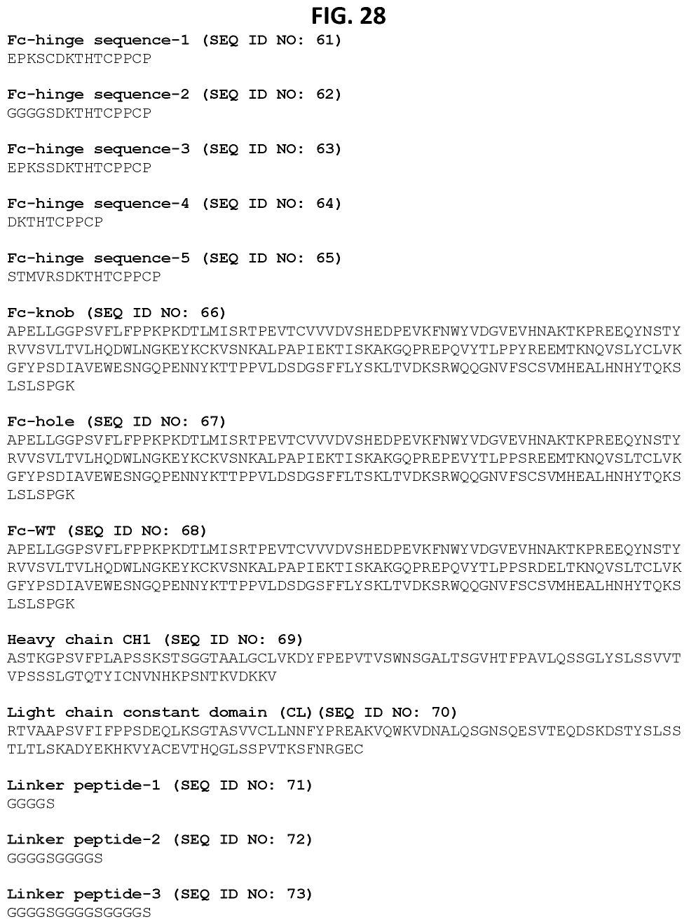

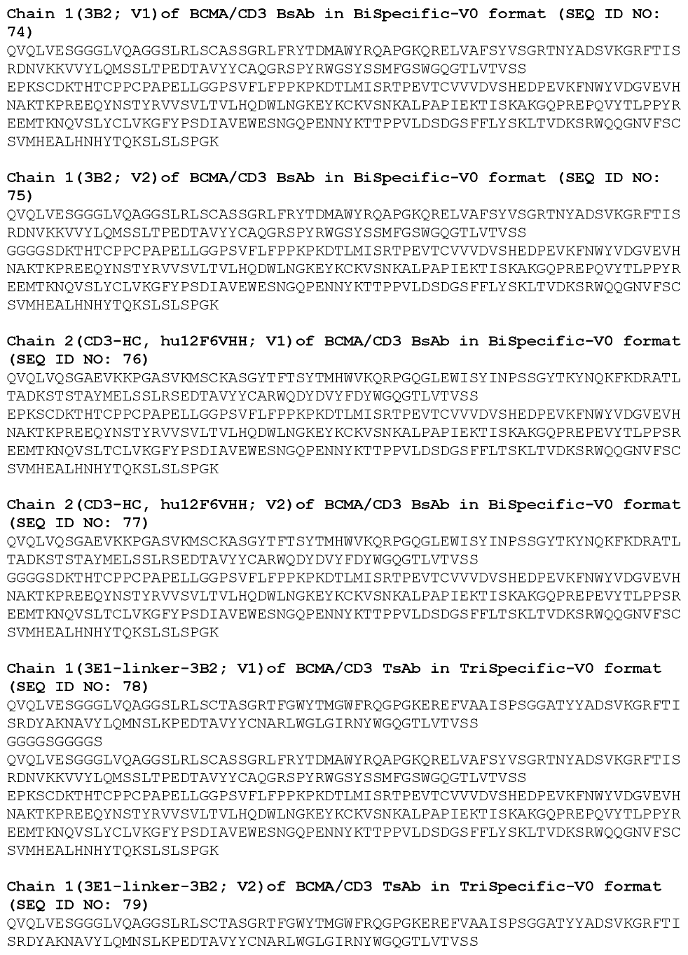

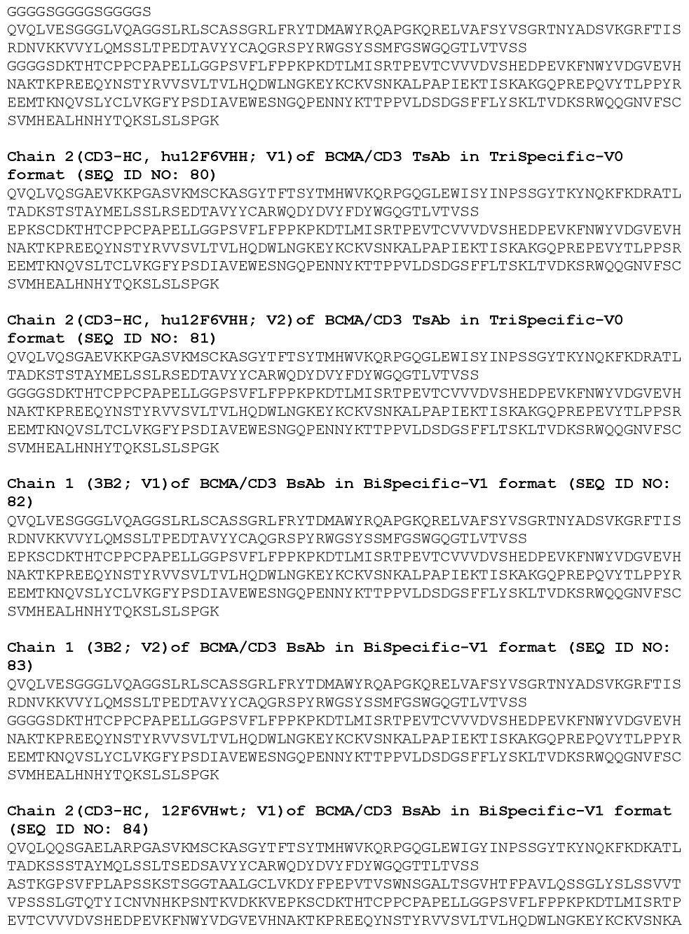

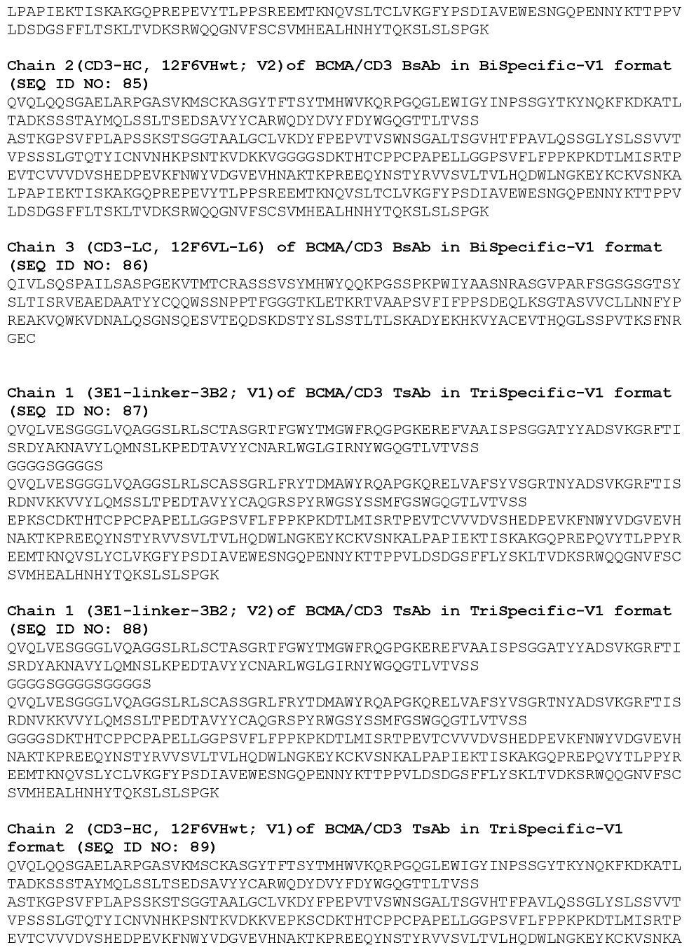

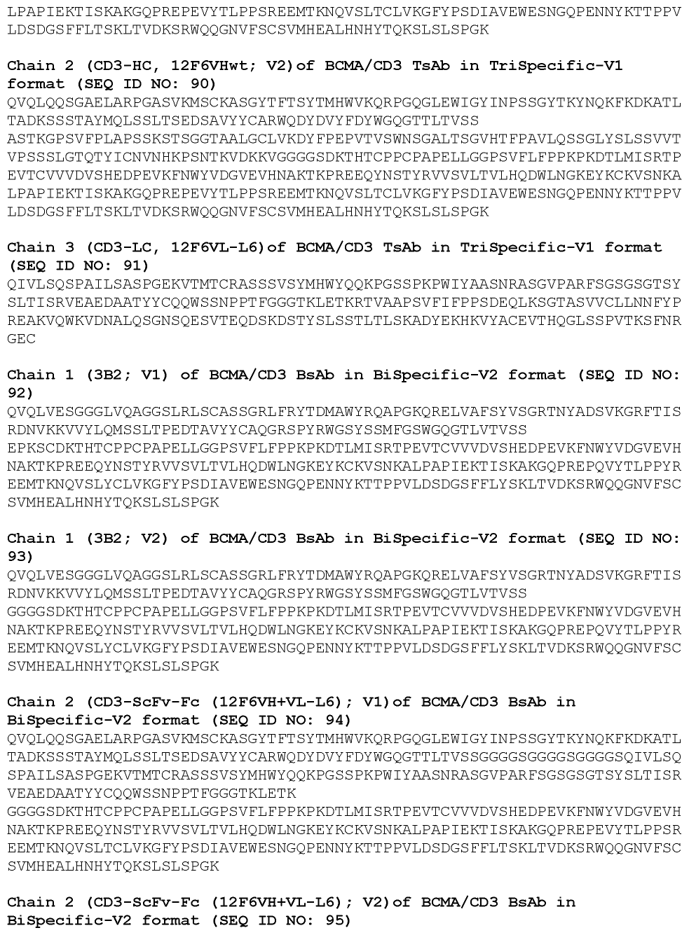

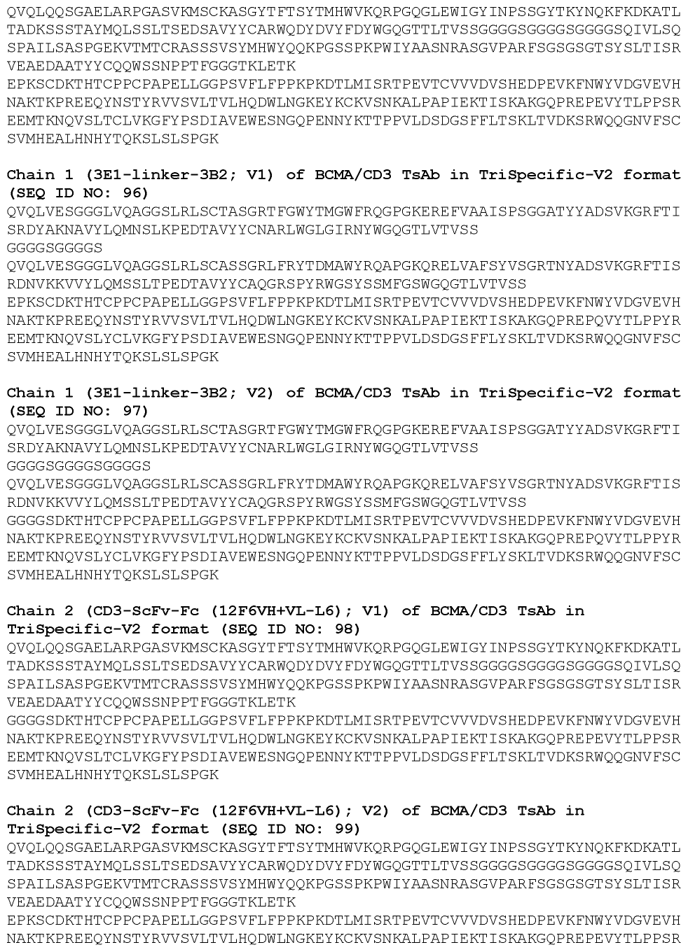

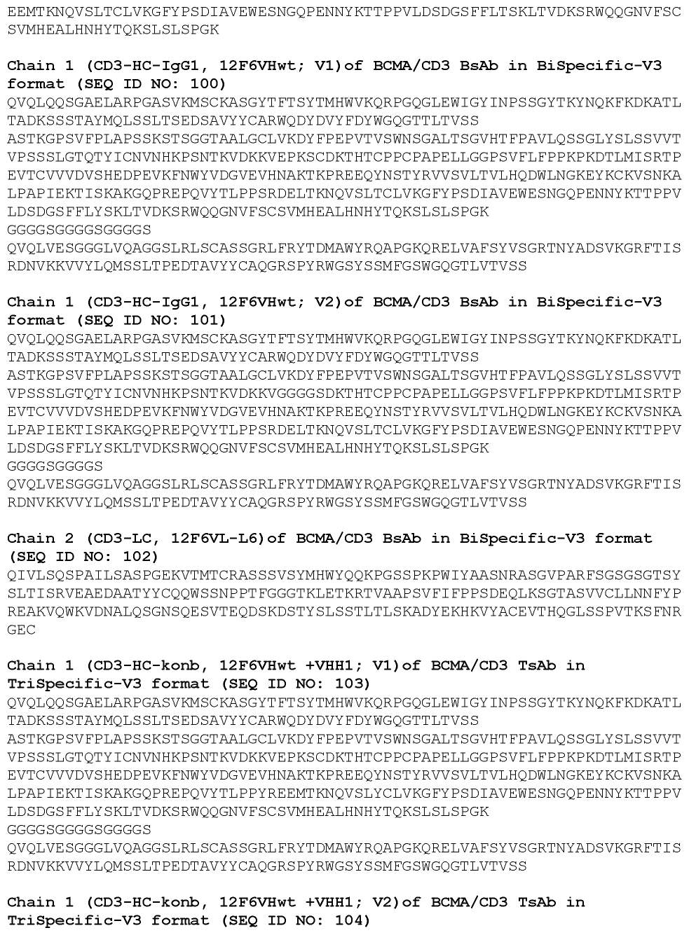

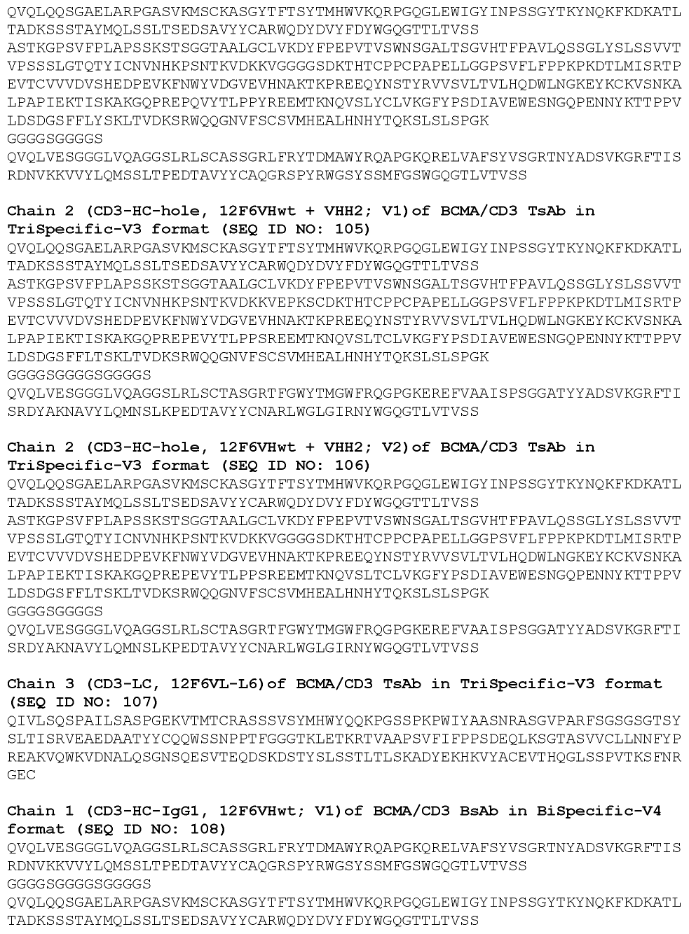

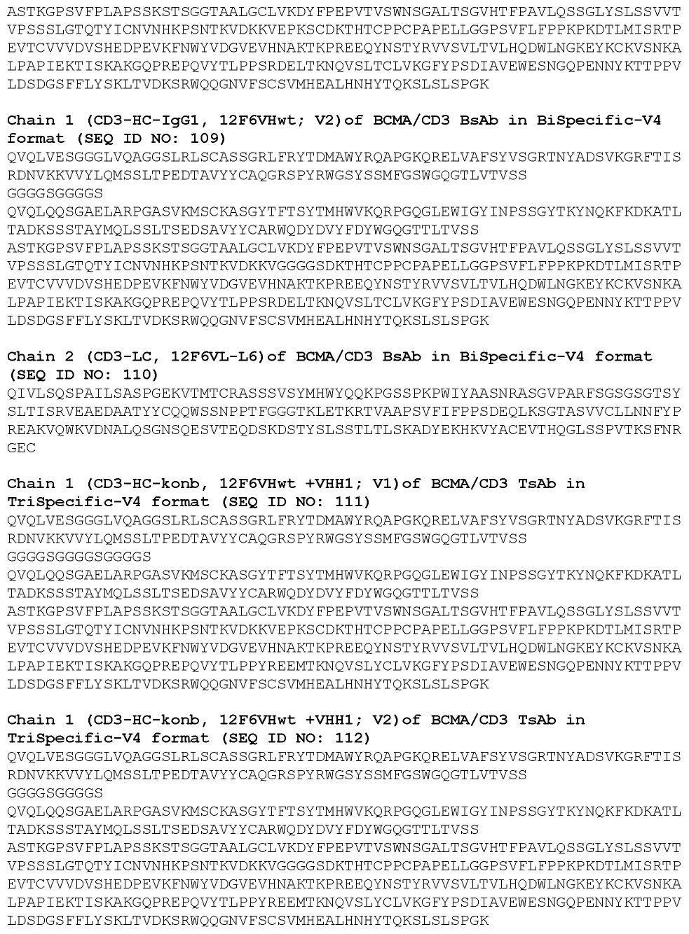

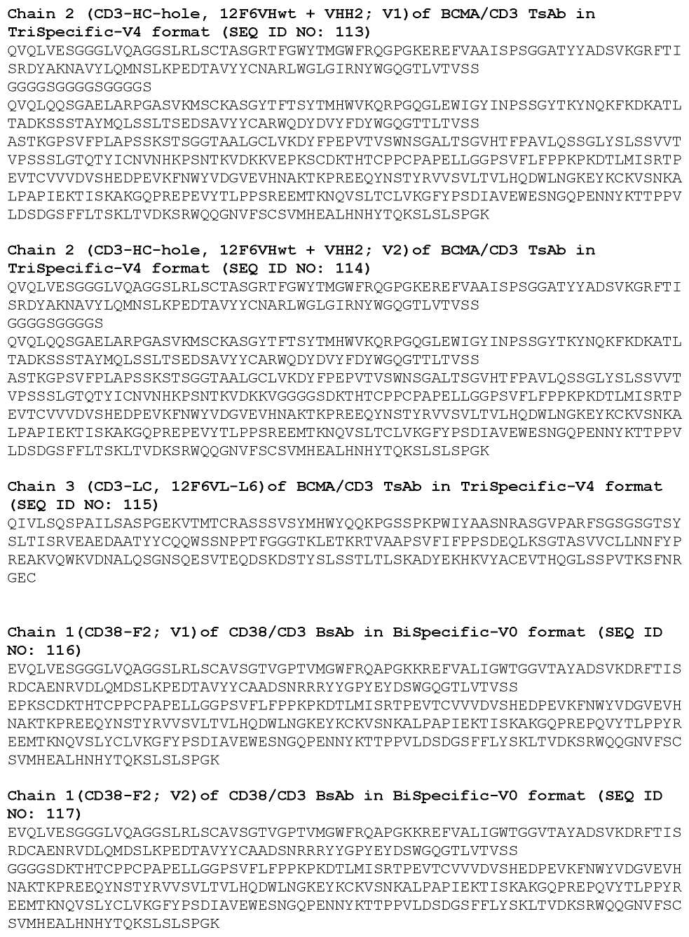

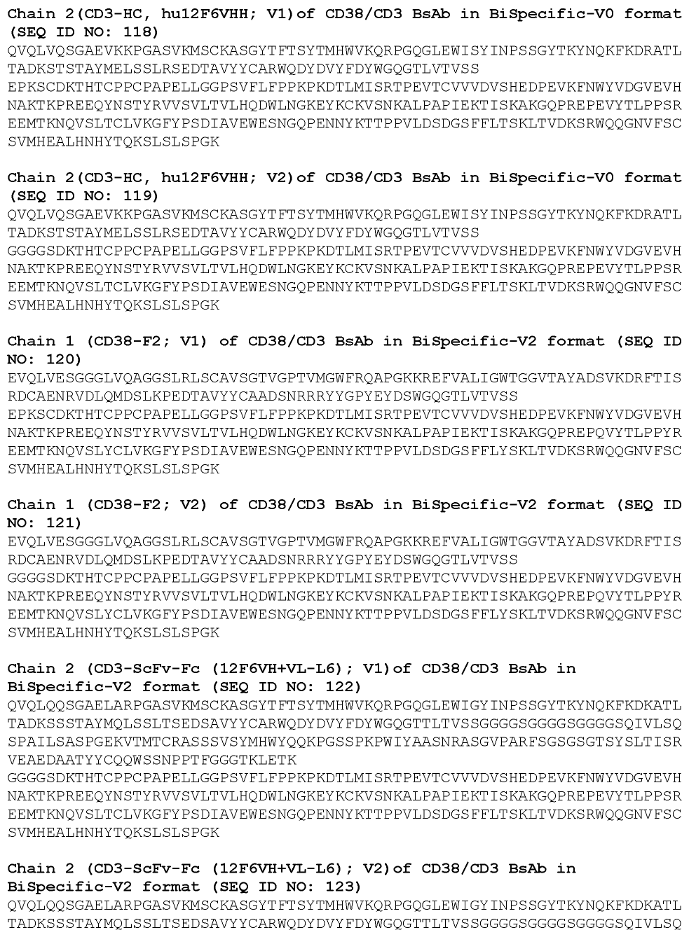

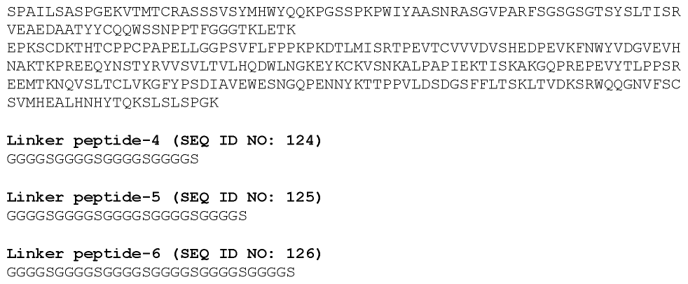

The disclosure also provides nucleic acid comprising a polynucleotide encoding a polypeptide comprising an immunoglobulin heavy-chain antibody variable domain (VHH). The VHH comprises CDRs as shown in FIG. 23, or has sequences as shown in FIG. 26.

The antibodies and antigen-binding fragments can also be antibody variants (including derivatives and conjugates) of antibodies or antibody fragments and multi-specific (e.g., bi-specific) antibodies or antibody fragments. Additional antibodies provided herein are polyclonal, monoclonal, multi-specific (multimeric, e.g., bi-specific), human antibodies, chimeric antibodies (e.g., human-mouse chimera), single-chain antibodies, intracellularly-made antibodies (i.e., intrabodies), and antigen-binding fragments thereof.

In some embodiments, the antibodies or antigen-binding fragments thereof comprises an Fc domain that can be originated from various types (e.g., IgG, IgE, IgM, IgD, IgA, and IgY), class (e.g., IgG1, IgG2, IgG3, IgG4, IgA1, and IgA2), or subclass. In some embodiments, the Fc domain is originated from an IgG antibody or antigen-binding fragment thereof. In some embodiments, the Fc domain comprises one, two, three, four, or more heavy chain constant regions.

The disclosure also provides antibodies or antigen-binding fragments thereof that bind to BCMA. In some embodiments, the antibody or antigen-binding fragment thereof comprises a heavy-chain antibody variable domain (VHH) CDR1 selected from SEQ ID NO: 1, 4, 7, or 10. In some embodiments, the antibody or antigen-binding fragment thereof comprises a heavy-chain antibody variable domain (VHH) CDR2 selected from SEQ ID NO: 2, 5, 8, or 11. In some embodiments, the antibody or antigen-binding fragment thereof comprises a heavy-chain antibody variable domain (VHH) CDR3 selected from SEQ ID NO: 3, 6, 9, or 12.

The disclosure also provides antibodies or antigen-binding fragments thereof that bind to CD38. In some embodiments, the antibody or antigen-binding fragment thereof comprises a heavy-chain antibody variable domain (VHH) CDR1 of SEQ ID NO: 13. In some embodiments, the antibody or antigen-binding fragment thereof comprises a heavy-chain antibody variable domain (VHH) CDR2 of SEQ ID NO: 14. In some embodiments, the antibody or antigen-binding fragment thereof comprises a heavy-chain antibody variable domain (VHH) CDR3 of SEQ ID NO: 15.

The disclosure also provides antibodies or antigen-binding fragments thereof that bind to CD3. In some embodiments, the antibody or antigen-binding fragment thereof comprises a heavy-chain antibody variable domain (VHH) CDR1 selected from SEQ ID NO: 16, 19, or 22. In some embodiments, the antibody or antigen-binding fragment thereof comprises a heavy-chain antibody variable domain (VHH) CDR2 selected from SEQ ID NO: 17, 20, or 23. In some embodiments, the antibody or antigen-binding fragment thereof comprises a heavy-chain antibody variable domain (VHH) CDR3 selected from SEQ ID NO: 18, 21, or 24.

### Anti-CD3 Antibodies and Antigen-Binding Fragments

The disclosure provides antibodies and antigen-binding fragments thereof that specifically bind to CD3. The antibodies and antigen-binding fragments described herein are capable of binding to CD3. These antibodies can be agonists or antagonists. In some embodiments, these antibodies can promote CD3-associated signaling pathway (e.g., antigen presenting to T cells) thus increase immune response. In some embodiments, these antibodies can initiate CMC or ADCC.

The disclosure provides e.g., anti-CD3 antibody 12F6, the chimeric antibodies thereof, and the humanized antibodies thereof. In some embodiments, the CDR sequences for 12F6, and 12F6 derived antibodies (e.g., humanized antibodies) include CDRs of the heavy chain variable domain, SEQ ID NOs: 25, 26, and 27. The VH with these VH CDRs can be paired with VLs with various different VL CDRs. In some embodiments, the CDR sequences for 12F6, and 12F6 derived antibodies (e.g., humanized antibodies) include CDRs of the light chain variable domain, SEQ ID NOs: 28, 29, and 30. In some embodiments, the CDR sequences for 12F6, and 12F6 derived antibodies (e.g., humanized antibodies) include CDRs of the light chain variable domain, SEQ ID NOs: 31, 32, and 33. In some embodiments, the CDR sequences for 12F6, and 12F6 derived antibodies (e.g., humanized antibodies) include CDRs of the light chain variable domain, SEQ ID NOs: 34, 35, and 36. In some embodiments, the CDR sequences for 12F6, and 12F6 derived antibodies (e.g., humanized antibodies) include CDRs of the light chain variable domain, SEQ ID NOs: 37, 38, and 39. In some embodiments, the CDR sequences for 12F6, and 12F6 derived antibodies (e.g., humanized antibodies) include CDRs of the light chain variable domain, SEQ ID NOs: 40, 41, and 42. In some embodiments, the CDR sequences for 12F6, and 12F6 derived antibodies (e.g., humanized antibodies) include CDRs of the light chain variable domain, SEQ ID NOs: 43, 44, and 45.

The amino acid sequences for heavy chain variable regions and light variable regions of the humanized antibodies are also provided. As there are different ways to humanize a mouse antibody (e.g., a sequence can be modified with different amino acid substitutions), the heavy chain and the light chain of an antibody can have more than one version of humanized sequences. In some embodiments, the humanized heavy chain variable region (VH) is at least 80%, 85%, 90%, 91%, 92%, 93%, 94%, 95%, 96%, 97%, 98%, or 99% identical to SEQ ID NO: 54. In some embodiments, the humanized light chain variable region (VL) is at least 80%, 85%, 90%, 91%, 92%, 93%, 94%, 95%, 96%, 97%, 98%, or 99% identical to any one of SEQ ID NOs: 55-60.

The amino acid sequences for the heavy chain variable region of 12F6 antibody are set forth in SEQ ID NO: 54. The amino acid sequences for the light chain variable regions of 12F6 antibody are set forth in SEQ ID NOs: 55-60. The heavy chain variable region sequence (SEQ ID NO: 54) can be paired with any of these light chain variable region sequences (SEQ ID NOs: 55-60).

Furthermore, in some embodiments, the antibodies or antigen-binding fragments thereof described herein can also contain one, two, or three heavy chain variable region CDRs selected from the group of SEQ ID NOs: 25-27; and/or one, two, or three light chain variable region CDRs selected from the group of SEQ ID NOs: 28-30, SEQ ID NOs: 31-33, SEQ ID NOs: 34-36, SEQ ID NOs: 37-39, SEQ ID NOs: 40-42, and SEQ ID NOs: 43-45.

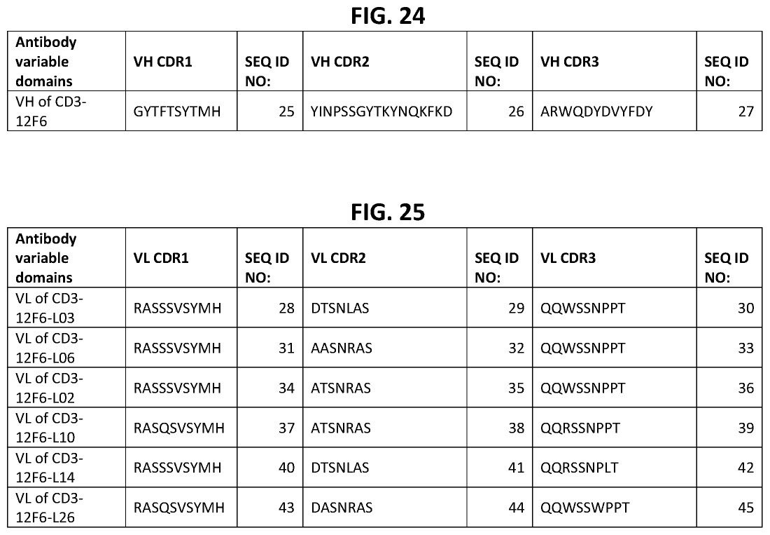

In some embodiments, the antibodies can have a heavy chain variable region (VH) comprising complementarity determining regions (CDRs) 1, 2, 3, wherein the CDR1 region comprises or consists of an amino acid sequence that is at least 80%, 85%, 90%, or 95% identical to a selected VH CDR1 amino acid sequence, the CDR2 region comprises or consists of an amino acid sequence that is at least 80%, 85%, 90%, or 95% identical to a selected VH CDR2 amino acid sequence, and the CDR3 region comprises or consists of an amino acid sequence that is at least 80%, 85%, 90%, or 95% identical to a selected VH CDR3 amino acid sequence, and a light chain variable region (VL) comprising CDRs 1, 2, 3, wherein the CDR1 region comprises or consists of an amino acid sequence that is at least 80%, 85%, 90%, or 95% identical to a selected VL CDR1 amino acid sequence, the CDR2 region comprises or consists of an amino acid sequence that is at least 80%, 85%, 90%, or 95% identical to a selected VL CDR2 amino acid sequence, and the CDR3 region comprises or consists of an amino acid sequence that is at least 80%, 85%, 90%, or 95% identical to a selected VL CDR3 amino acid sequence. The selected VH CDRs 1, 2, 3 amino acid sequences and the selected VL CDRs, 1, 2, 3 amino acid sequences are shown in FIG. 24 (VH CDR) and FIG. 25 (VL CDR).

In some embodiments, the antibody or an antigen-binding fragment described herein can contain a heavy chain variable domain containing one, two, or three of the CDRs of SEQ ID NO: 25 with zero, one or two amino acid insertions, deletions, or substitutions; SEQ ID NO: 26 with zero, one or two amino acid insertions, deletions, or substitutions; SEQ ID NO: 27 with zero, one or two amino acid insertions, deletions, or substitutions.

In some embodiments, the antibody or an antigen-binding fragment described herein can contain a light chain variable domain containing one, two, or three of the CDRs of SEQ ID NO: 28 with zero, one or two amino acid insertions, deletions, or substitutions; SEQ ID NO: 29 with zero, one or two amino acid insertions, deletions, or substitutions; SEQ ID NO: 30 with zero, one or two amino acid insertions, deletions, or substitutions.

In some embodiments, the antibody or an antigen-binding fragment described herein can contain a light chain variable domain containing one, two, or three of the CDRs of SEQ ID NO: 31 with zero, one or two amino acid insertions, deletions, or substitutions; SEQ ID NO: 32 with zero, one or two amino acid insertions, deletions, or substitutions; SEQ ID NO: 33 with zero, one or two amino acid insertions, deletions, or substitutions.

In some embodiments, the antibody or an antigen-binding fragment described herein can contain a light chain variable domain containing one, two, or three of the CDRs of SEQ ID NO: 34 with zero, one or two amino acid insertions, deletions, or substitutions; SEQ ID NO: 35 with zero, one or two amino acid insertions, deletions, or substitutions; SEQ ID NO: 36 with zero, one or two amino acid insertions, deletions, or substitutions.

In some embodiments, the antibody or an antigen-binding fragment described herein can contain a light chain variable domain containing one, two, or three of the CDRs of SEQ ID NO: 37 with zero, one or two amino acid insertions, deletions, or substitutions; SEQ ID NO: 38 with zero, one or two amino acid insertions, deletions, or substitutions; SEQ ID NO: 39 with zero, one or two amino acid insertions, deletions, or substitutions.

In some embodiments, the antibody or an antigen-binding fragment described herein can contain a light chain variable domain containing one, two, or three of the CDRs of SEQ ID NO: 40 with zero, one or two amino acid insertions, deletions, or substitutions; SEQ ID NO: 41 with zero, one or two amino acid insertions, deletions, or substitutions; SEQ ID NO: 42 with zero, one or two amino acid insertions, deletions, or substitutions.

In some embodiments, the antibody or an antigen-binding fragment described herein can contain a light chain variable domain containing one, two, or three of the CDRs of SEQ ID NO: 43 with zero, one or two amino acid insertions, deletions, or substitutions; SEQ ID NO: 44 with zero, one or two amino acid insertions, deletions, or substitutions; SEQ ID NO: 45 with zero, one or two amino acid insertions, deletions, or substitutions.

The insertions, deletions, and substitutions can be within the CDR sequence, or at one or both terminal ends of the CDR sequence. In some embodiments, the CDR is determined based on Kabat numbering scheme. In some embodiments, the CDR is determined based on Chothia numbering scheme. In some embodiments, the CDR is determined based on a combination numbering scheme.

The disclosure also provides antibodies or antigen-binding fragments thereof that bind to CD3. The antibodies or antigen-binding fragments thereof contain a heavy chain variable region (VH) comprising or consisting of an amino acid sequence that is at least 80%, 85%, 90%, or 95% identical to a selected VH sequence, and a light chain variable region (VL) comprising or consisting of an amino acid sequence that is at least 80%, 85%, 90%, or 95% identical to a selected VL sequence. In some embodiments, the selected VH sequence is SEQ ID NO: 54, and the selected VL sequence is SEQ ID NO: 55, 56, 57, 58, 59, or 60.

The disclosure also provides nucleic acid comprising a polynucleotide encoding a polypeptide comprising an immunoglobulin heavy chain or an immunoglobulin light chain. The immunoglobulin heavy chain or immunoglobulin light chain comprises CDRs as shown in FIG. 24 and FIG. 25, respectively, or have sequences as shown in FIG. 24. When the polypeptides are paired with corresponding polypeptide (e.g., a corresponding heavy chain variable region or a corresponding light chain variable region), the paired polypeptides bind to CD3 (e.g., human CD3).

The anti-CD3 antibodies and antigen-binding fragments can also be antibody variants (including derivatives and conjugates) of antibodies or antibody fragments and multi-specific (e.g., bi-specific) antibodies or antibody fragments. Additional antibodies provided herein are polyclonal, monoclonal, multi-specific (multimeric, e.g., bi-specific), human antibodies, chimeric antibodies (e.g., human-mouse chimera), single-chain antibodies, intracellularly-made antibodies (i.e., intrabodies), and antigen-binding fragments thereof. The antibodies or antigen-binding fragments thereof can be of any type (e.g., IgG, IgE, IgM, IgD, IgA, and IgY), class (e.g., IgG1, IgG2, IgG3, IgG4, IgA1, and IgA2), or subclass. In some embodiments, the antibody or antigen-binding fragment thereof is an IgG antibody or antigen-binding fragment thereof.

Fragments of antibodies are suitable for use in the methods provided so long as they retain the desired affinity and specificity of the full-length antibody. Thus, a fragment of an antibody that binds to CD3 will retain an ability to bind to CD3.

In one aspect, the disclosure is related to a nucleic acid comprising a polynucleotide encoding a polypeptide comprising:
(1) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 25, 26, and 27, respectively, and wherein the VH, when paired with a light chain variable region (VL) comprising the amino acid sequence set forth in SEQ ID NO: 55, 56, 57, 58, 59, or 60 binds to CD3;
(2) an immunoglobulin light chain or a fragment thereof comprising a VL comprising CDRs 1, 2, and 3 comprising the amino acid sequences set forth in SEQ ID NOs: 28, 29, and 30, respectively, and wherein the VL, when paired with a VH comprising the amino acid sequence set forth in SEQ ID NO: 54 binds to CD3;
(3) an immunoglobulin light chain or a fragment thereof comprising a VL comprising CDRs 1, 2, and 3 comprising the amino acid sequences set forth in SEQ ID NOs: 31, 32, and 33, respectively, and wherein the VL, when paired with a VH comprising the amino acid sequence set forth in SEQ ID NO: 54 binds to CD3;
(4) an immunoglobulin light chain or a fragment thereof comprising a VL comprising CDRs 1, 2, and 3 comprising the amino acid sequences set forth in SEQ ID NOs: 34, 35, and 36, respectively, and wherein the VL, when paired with a VH comprising the amino acid sequence set forth in SEQ ID NO: 54 binds to CD3;
(5) an immunoglobulin light chain or a fragment thereof comprising a VL comprising CDRs 1, 2, and 3 comprising the amino acid sequences set forth in SEQ ID NOs: 37, 38, and 39, respectively, and wherein the VL, when paired with a VH comprising the amino acid sequence set forth in SEQ ID NO: 54 binds to CD3;
(6) an immunoglobulin light chain or a fragment thereof comprising a VL comprising CDRs 1, 2, and 3 comprising the amino acid sequences set forth in SEQ ID NOs: 40, 41, and 42, respectively, and wherein the VL, when paired with a VH comprising the amino acid sequence set forth in SEQ ID NO: 54 binds to CD3; or
(7) an immunoglobulin light chain or a fragment thereof comprising a VL comprising CDRs 1, 2, and 3 comprising the amino acid sequences set forth in SEQ ID NOs: 43, 44, and 45, respectively, and wherein the VL, when paired with a VH comprising the amino acid sequence set forth in SEQ ID NO: 54 binds to CD3.

In some embodiments, the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 25, 26, and 27, respectively.

In some embodiments, the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 28, 29, and 30, respectively.

In some embodiments, the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 31, 32, and 33, respectively.

In some embodiments, the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 34, 35, and 36, respectively.

In some embodiments, the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 37, 38, and 39, respectively.

In some embodiments, the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 40, 41, and 42, respectively.

In some embodiments, the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 43, 44, and 45, respectively.

In some embodiments, the VH when paired with a VL specifically binds to human CD3, or the VL when paired with a VH specifically binds to human CD3.

In some embodiments, the immunoglobulin heavy chain or the fragment thereof is a humanized immunoglobulin heavy chain or a fragment thereof, and the immunoglobulin light chain or the fragment thereof is a humanized immunoglobulin light chain or a fragment thereof.

In some embodiments, the nucleic acid encodes a single-chain variable fragment (scFv). In some embodiments, the nucleic acid is cDNA.

In one aspect, the disclosure is related to a vector comprising one or more of the nucleic acids described herein. In one aspect, the disclosure is related to a vector comprising two of the nucleic acids described herein, wherein the vector encodes the VL region and the VH region that together bind to CD3. In one aspect, the disclosure is related to a pair of vectors, wherein each vector comprises one of the nucleic acids described herein, wherein together the pair of vectors encodes the VL region and the VH region that together bind to CD3.

In one aspect, the disclosure is related to a cell comprising the vector or the pair of vectors described herein. In one aspect, the disclosure is related to two of the nucleic acids described herein.

In some embodiments, the two nucleic acids together encode the VL region and the VH region that together bind to CD3.

Imbalanced Multi-Specific Antibodies that Bind to T Cell Specific Antigen and Cancer Antigens

Multispecific antibodies (e.g., bispecific antibody) with a T cell specific antigen (e.g., CD3, CD4, or CD8) binding site that can recruit and activate T cells. Because an antibody's effector function such as ADCC and CDC have been shown to play a critical role in cancer cell killing, “safely” maintaining an antibody's effector function would expand the mechanisms of action of an therapeutic antibody as well as improve the antibody's cancer killing function. To “safely” maintain the effector functions and expand the applications of these bispecific antibodies, various multispecific antibodies have been developed.

In an exemplary design shown in FIG. 19, the first antigen binding region targets a cancer antigen (e.g., BCMA or CD38), and the second antigen binding region targets a T cell specific antigen, i.e., CD3, to recruit T cell to attack cancer with the cancer antigen. The term “cancer antigen” refers to antigens that are primarily expressed on cancer cell surfaces. These antigens can be used to identify cancer or tumor cells. Normal cells rarely express cancer antigens.

An antibody with high affinity to CD3 can trigger T-cell signaling, and cause undesirable immune response. Thus, a low affinity (e.g., KD can be greater than 10⁻⁵ M, 10⁻⁶ M, 10⁻⁷ M, 10⁻⁸ M, or 10⁻⁹ M) to CD3 is required to reduce the risk of triggering T-cell signaling by CD3 while “safely” maintaining the antibody's effector function. As used herein, the term “safely maintaining the antibody's effector function” means that the antibody does not induce ADCC or CDC on normal cells (e.g., non-cancer cells). When multiple bispecific antibodies are presented on a target cancer cells (e.g., in a cluster) and bridge the interaction between cancer cell and T cell, these bispecific antibodies can trigger T-cell signaling though CD3 in a multivalent fashion, and the activated T cells will then kill the target cancer cells. Because the multispecific antibody applies different mechanism of action to treat cancer compared to therapeutic antibodies that target a cancer specific antigen alone, in some embodiments, it can be used as an alternative therapy for therapeutic monoclonal antibodies that target a cancer specific antigen, especially for those cancers which don not respond well to therapeutic monoclonal antibodies that only targets a cancer specific antigen.

In some embodiments, multi-specific (e.g., trispecific) antibodies are designed that include an additional antigen binding region that targets a cancer antigen (e.g., BCMA or CD38). The multispecific (e.g., bispecific and trispecific) antibodies are described below.

### (1) BCMA CD3 Multispecific Antibody

BCMA (B-cell maturation antigen, BCM, or tumor necrosis factor receptor superfamily member 17 (TNFRSF17)) is a cancer antigen and is expressed on e.g., blood cancer cells. The present disclosure provides multi-specific (e.g., bispecific or trispecific) antibodies that bind to both BCMA and CD3. The bispecific or trispecific antibodies can be used to treat BCMA positive cancers (e.g., multiple myeloma or hematological malignancy) in a subject.

The BCMA/CD3 bispecific and trispecific antibodies with specific structures are described below.

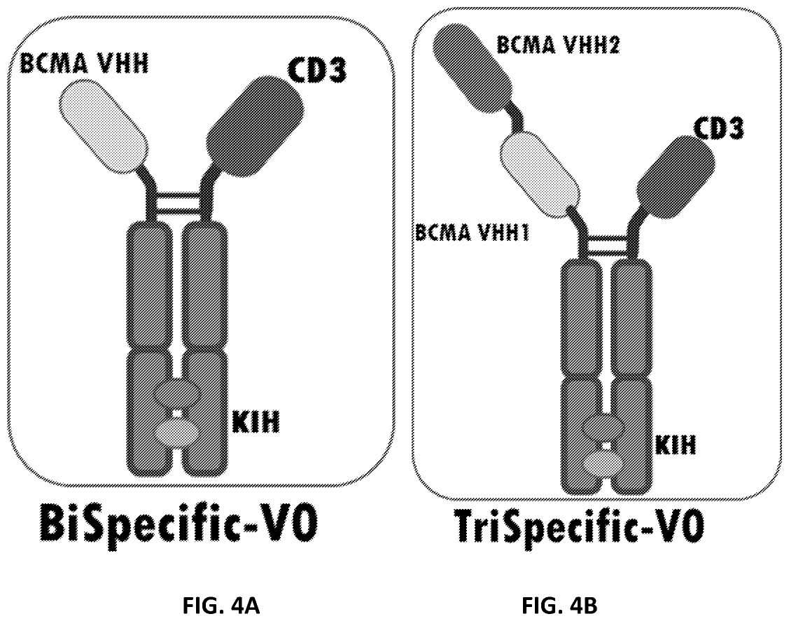

As shown in FIG. 4A, a BCMA/CD3 bispecific antibody can be prepared to have a BiSpecific-V0 structure. Specifically, the BCMA/CD3 bispecific antibody comprises (a) a first polypeptide comprising from N-terminus to C-terminus: a first heavy-chain antibody variable domain (VHH), a first hinge region, a first Fc region; and (b) a second polypeptide comprising from N-terminus to C-terminus: a second VHH, a second hinge region, and a second Fc region. In some embodiments, the first VHH specifically binds to BCMA, and the second VHH specifically binds to CD3.

In some embodiments, the first hinge region and/or the second hinge region comprise a sequence that is at least 80%, 85%, 90%, 95%, or 100% identical to any one of SEQ ID NOs: 61-65. In some embodiments, the first Fc region and/or the second Fc region comprise a sequence that is at least 80%, 85%, 90%, 95%, or 100% identical to SEQ ID NO: 66 or 67.

In some embodiments, the BCMA/CD3 bispecific antibody comprises knob-into-hole mutations. In some embodiments, the Fc region is an IgG1 Fc region. In some embodiments, the first polypeptide comprises a sequence that is at least 80%, 85%, 90%, 95%, or 100% identical to SEQ ID NO: 74 or 75. In some embodiments, the second polypeptide comprises a sequence that is at least 80% identical to SEQ ID NOs: 76 or 77.

As shown in FIG. 4B, a BCMA/CD3 trispecific antibody can be prepared to have a TriSpecific-V0 structure. Specifically, the first polypeptide as described in the BiSpecific-V0 structure can further include a third VHH that specifically binds to BCMA, wherein the third VHH is linked to the N-terminus of the first VHH via a linker peptide sequence. In some embodiments, the linker peptide sequence comprises a sequence that is at least 80%, 85%, 90%, 95%, or 100% identical to any one SEQ ID NOs: 71-73 and 124-126. In some embodiments, the linker peptide sequence comprises a sequence that is at least 80%, 85%, 90%, 95%, or 100% identical to SEQ ID NO: 72 or 73. In some embodiments, the linker peptide sequence comprises a sequence that is at least 80%, 85%, 90%, 95%, or 100% identical to one or more (e.g., 1, 2, 3, 4, 5, 6, 7, or 8) repeats of GGGGS (SEQ ID NO: 71).

In some embodiments, the first polypeptide comprises a sequence that is at least 80%, 85%, 90%, 95%, or 100% identical to SEQ ID NO: 78 or 79. In some embodiments, the second polypeptide comprises a sequence that is at least 80% identical to SEQ ID NO: 80 or 81.

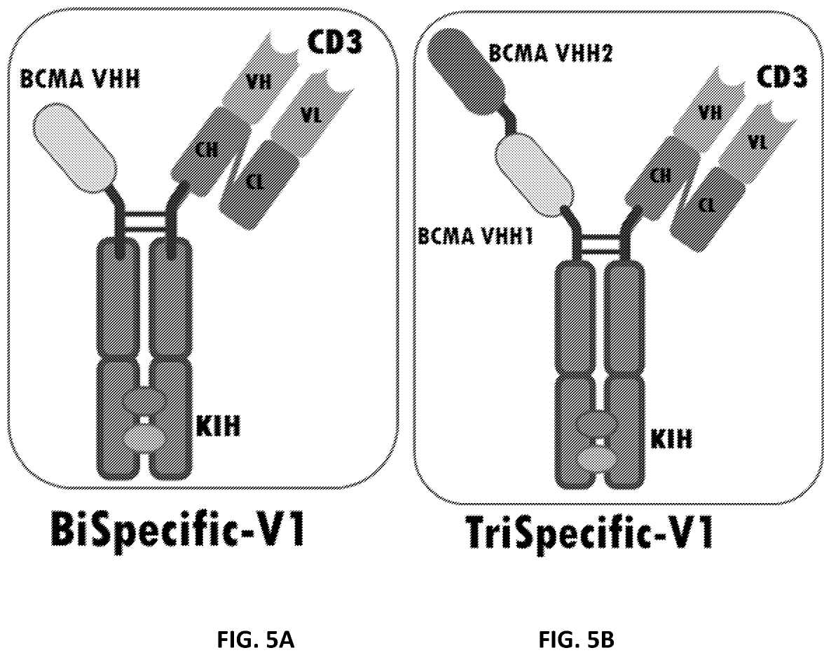

As shown in FIG. 5A, a BCMA/CD3 bispecific antibody can be prepared to have a BiSpecific-V1 structure. Specifically, the BCMA/CD3 bispecific antibody comprises (a) a first polypeptide comprising from N-terminus to C-terminus: a first heavy-chain antibody variable domain (VHH), a first hinge region, a first Fc region; (b) a second polypeptide comprising from N-terminus to C-terminus: a heavy chain variable region (VH), a second hinge region, and a second Fc region; and (c) a third polypeptide comprising a light chain variable region (VL). In some embodiments, the first VHH specifically binds to BCMA, wherein the VH and the VL associate with each other and specifically bind to CD3.

In some embodiments, the first hinge region and/or the second hinge region comprise a sequence that is at least 80%, 85%, 90%, 95%, or 100% identical to any one of SEQ ID NOs: 61-65. In some embodiments, the first Fc region and/or the second Fc region comprise a sequence that is at least 80%, 85%, 90%, 95%, or 100% identical to SEQ ID NO: 66 or 67.

In some embodiments, the BCMA/CD3 bispecific antibody comprises knob-into-hole mutations. In some embodiments, the Fc region is an IgG1 Fc region. In some embodiments, the first polypeptide comprises a sequence that is at least 80%, 85%, 90%, 95%, or 100% identical to SEQ ID NO: 82 or 83. In some embodiments, the second polypeptide comprises a sequence that is at least 80%, 85%, 90%, 95%, or 100% identical to SEQ ID NO: 84 or 85. In some embodiments, the third polypeptide comprises a sequence that is at least 80%, 85%, 90%, 95%, or 100% identical to SEQ ID NO: 86.

As shown in FIG. 5B, a BCMA/CD3 trispecific antibody can be prepared to have a TriSpecific-V1 structure. Specifically, the first polypeptide as described in the BiSpecific-V1 structure can further include a second VHH that specifically binds to BCMA, wherein the second VHH is linked to the N-terminus of the first VHH via a linker peptide sequence. In some embodiments, the linker peptide sequence comprises a sequence that is at least 80%, 85%, 90%, 95%, or 100% identical to any one SEQ ID NOs: 71-73 and 124-126. In some embodiments, the linker peptide sequence comprises a sequence that is at least 80% identical to SEQ ID NO: 72 or 73. In some embodiments, the linker peptide sequence comprises a sequence that is at least 80%, 85%, 90%, 95%, or 100% identical to one or more (e.g., 1, 2, 3, 4, 5, 6, 7, or 8) repeats of GGGGS (SEQ ID NO: 71).

In some embodiments, the first polypeptide comprises a sequence that is at least 80%, 85%, 90%, 95%, or 100% identical to SEQ ID NO: 87 or 88. In some embodiments, the second polypeptide comprises a sequence that is at least 80%, 85%, 90%, 95%, or 100% identical to SEQ ID NO: 89 or 90. In some embodiments, the third polypeptide comprises a sequence that is at least 80%, 85%, 90%, 95%, or 100% identical to SEQ ID NO: 91.

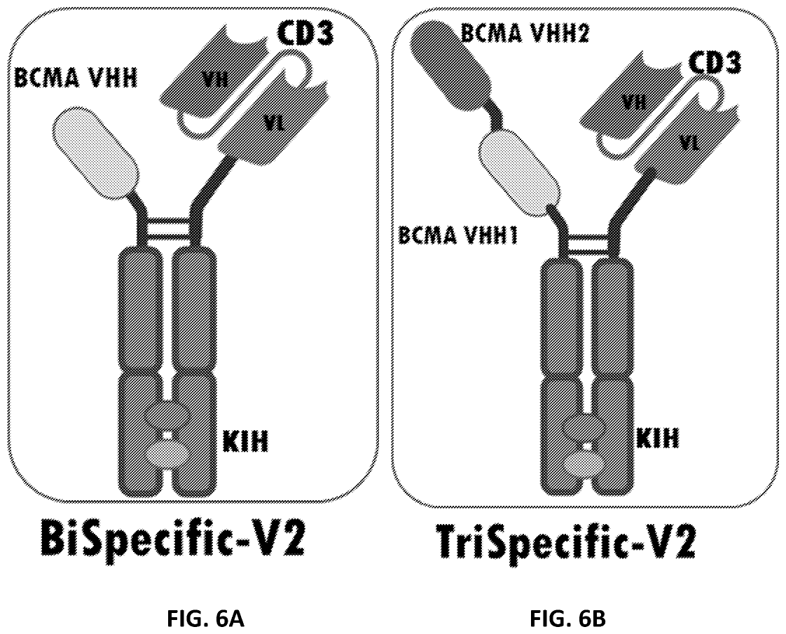

As shown in FIG. 6A, a BCMA/CD3 bispecific antibody can be prepared to have a BiSpecific-V2 structure. Specifically, the BCMA/CD3 bispecific antibody comprises (a) a first polypeptide comprising from N-terminus to C-terminus: a first heavy-chain antibody variable domain (VHH), a first hinge region, a first Fc region; and (b) a second polypeptide comprising from N-terminus to C-terminus: a single-chain variable fragment (scFv), a second hinge region, and a second Fc region.

In some embodiments, the first VHH specifically binds to BCMA. In some embodiments, the scFv comprises a heavy chain variable region (VH), a first linker peptide sequence, and a light chain variable region (VL). In some embodiments, the VH and the VL associate with each other and specifically bind to CD3.

In some embodiments, the first linker peptide sequence comprises a sequence that is at least 80%, 85%, 90%, 95%, or 100% identical to any one SEQ ID NOs: 71-73 and 124-126. In some embodiments, the first linker peptide sequence comprises a sequence that is at least 80%, 85%, 90%, 95%, or 100% identical to SEQ ID NO: 72 or 73. In some embodiments, the first hinge region and/or the second hinge region comprise a sequence that is at least 80%, 85%, 90%, 95%, or 100% identical to any one of SEQ ID NOs: 61-65. In some embodiments, the first Fc region and/or the second Fc region comprise a sequence that is at least 80%, 85%, 90%, 95%, or 100% identical to SEQ ID NO: 66 or 67.

In some embodiments, the BCMA/CD3 bispecific antibody comprises knob-into-hole mutations. In some embodiments, the Fc region is an IgG1 Fc region. In some embodiments, the first polypeptide comprises a sequence that is at least 80%, 85%, 90%, 95%, or 100% identical to SEQ ID NO: 92 or 93. In some embodiments, the second polypeptide comprises a sequence that is at least 80%, 85%, 90%, 95%, or 100% identical to SEQ ID NO: 94 or 95.

As shown in FIG. 6B, a BCMA/CD3 trispecific antibody can be prepared to have a TriSpecific-V2 structure. Specifically, the first polypeptide as described in the BiSpecific-V2 structure can further include a second VHH that specifically binds to BCMA, wherein the second VHH is linked to the N-terminus of the first VHH via a second linker peptide sequence.

In some embodiments, the second linker peptide sequence comprises a sequence that is at least 80%, 85%, 90%, 95%, or 100% identical to any one SEQ ID NOs: 71-73 and 124-126. In some embodiments, the second linker peptide sequence comprises a sequence that is at least 80%, 85%, 90%, 95%, or 100% identical to SEQ ID NO: 72 or 73. In some embodiments, the first and/or the second linker peptide sequence comprise a sequence that is at least 80%, 85%, 90%, 95%, or 100% identical to one or more (e.g., 1, 2, 3, 4, 5, 6, 7, or 8) repeats of GGGGS (SEQ ID NO: 71).

In some embodiments, the first polypeptide comprises a sequence that is at least 80%, 85%, 90%, 95%, or 100% identical to SEQ ID NO: 96 or 97. In some embodiments, the second polypeptide comprises a sequence that is at least 80% identical to SEQ ID NO: 98 or 99.

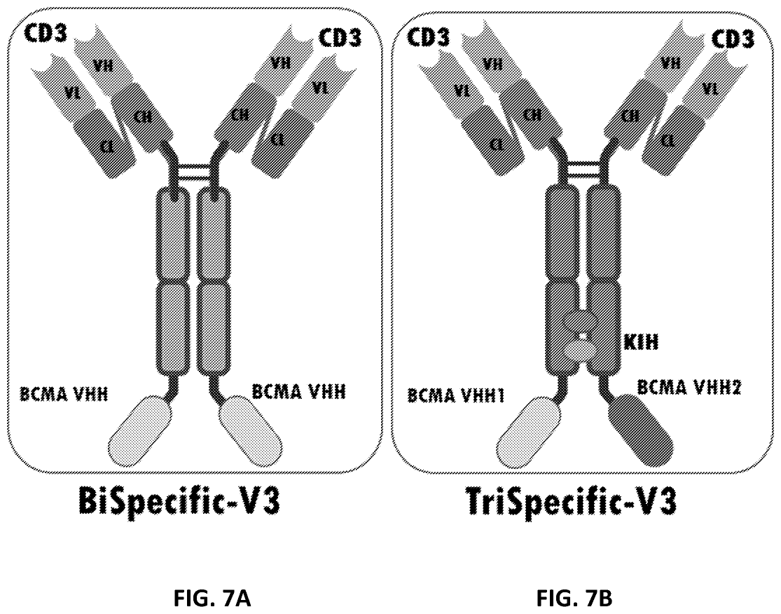

As shown in FIG. 7A, a BCMA/CD3 bispecific antibody can be prepared to have a BiSpecific-V3 structure. Specifically, the BCMA/CD3 bispecific antibody comprises (a) a first polypeptide comprising from N-terminus to C-terminus: a first heavy chain variable region (VH1), a first hinge region, a first Fc region; a first linker peptide sequence, and a first heavy-chain antibody variable domain (VHH1); (b) a second polypeptide comprising a first light chain variable region (VL1); (c) a third polypeptide comprising from N-terminus to C-terminus: a second heavy chain variable region (VH2), a second hinge region, a second Fc region, a second linker peptide sequence, and a second heavy-chain antibody variable domain (VHH2); and (d) a fourth polypeptide comprising a second light chain variable region (VL2). In some embodiments, the VHH1 and/or the VHH2 specifically bind to BCMA. In some embodiments, the VH1 and the VL1 associate with each other and specifically bind to CD3. In some embodiments, the VH2 and the VL2 associate with each other and specifically bind to CD3.

In some embodiments, the first linker peptide sequence and/or the second linker peptide sequence comprises a sequence that is at least 80%, 85%, 90%, 95%, or 100% identical to any one SEQ ID NOs: 71-73 and 124-126. In some embodiments, the first linker peptide sequence and/or the second linker peptide sequence comprises a sequence that is at least 80%, 85%, 90%, 95%, or 100% identical to SEQ ID NO: 72 or 73. In some embodiments, the first and/or the second linker peptide sequence comprise a sequence that is at least 80%, 85%, 90%, 95%, or 100% identical to one or more (e.g., 1, 2, 3, 4, 5, 6, 7, or 8) repeats of GGGGS (SEQ ID NO: 71).

In some embodiments, the first hinge region and/or the second hinge regions comprise a sequence that is at least 80%, 85%, 90%, 95%, or 100% identical to any one of SEQ ID NOs: 61-65.

In some embodiments, sequences of the VHH1 and the VHH2 are identical. In some embodiments, the first Fc region and/or the second Fc region comprise a sequence that is at least 80%, 85%, 90%, 95%, or 100% identical to SEQ ID NO: 68. In some embodiments, the first polypeptide comprises a sequence that is at least 80%, 85%, 90%, 95%, or 100% identical to SEQ ID NO: 100 or 101. In some embodiments, the second polypeptide comprises a sequence that is at least 80%, 85%, 90%, 95%, or 100% identical to SEQ ID NO: 102. In some embodiments, the third polypeptide comprises a sequence that is at least 80%, 85%, 90%, 95%, or 100% identical to SEQ ID NO: 100 or 101. In some embodiments, the fourth polypeptide comprises a sequence that is at least 80%, 85%, 90%, 95%, or 100% identical to SEQ ID NO: 102.

As shown in FIG. 7B, a BCMA/CD3 trispecific antibody can be prepared to have a TriSpecific-V3 structure. In some embodiments, sequences of the VHH1 and the VHH2 as described in the BiSpecific-V3 structure are different. In some embodiments, the first Fc region and/or the second Fc region comprise a sequence that is at least 80%, 85%, 90%, 95%, or 100% identical to SEQ ID NO: 66 or 67. In some embodiments, the first polypeptide comprises a sequence that is at least 80%, 85%, 90%, 95%, or 100% identical to SEQ ID NO: 103 or 104. In some embodiments, the second polypeptide comprises a sequence that is at least 80%, 85%, 90%, 95%, or 100% identical to SEQ ID NO: 107. In some embodiments, the third polypeptide comprises a sequence that is at least 80%, 85%, 90%, 95%, or 100% identical to SEQ ID NO: 105 or 106. In some embodiments, the fourth polypeptide comprises a sequence that is at least 80%, 85%, 90%, 95%, or 100% identical to SEQ ID NO: 107.

In some embodiments, the Fc region is an IgG1 (e.g., human IgG1) Fc region. In some embodiments, the BCMA/CD3 bispecific or trispecific antibody described herein comprises knob-into-hole mutations.

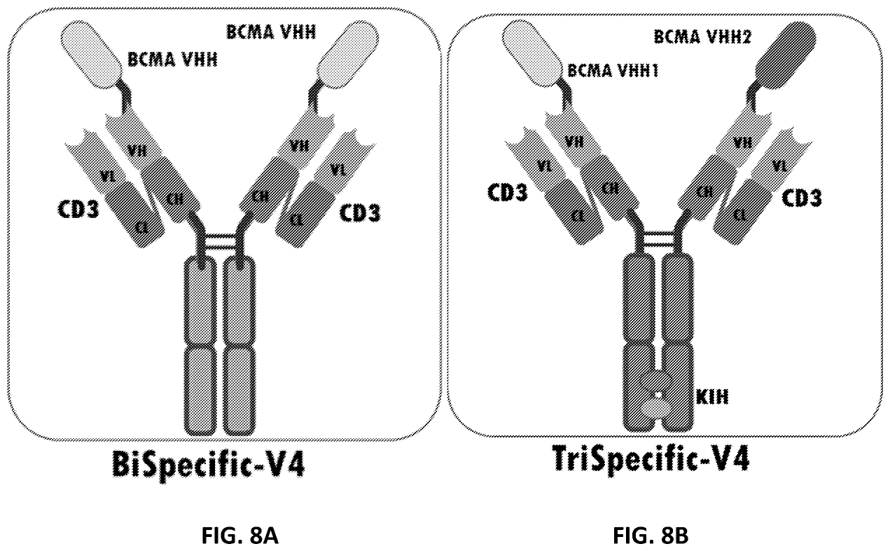

As shown in FIG. 8A, a BCMA/CD3 bispecific antibody can be prepared to have a BiSpecific-V4 structure. Specifically, the BCMA/CD3 bispecific antibody comprises (a) a first polypeptide comprising from N-terminus to C-terminus: a first heavy-chain antibody variable domain (VHH1), a first linker peptide sequence, a first heavy chain variable region (VH1), a first hinge region, and a first Fc region; (b) a second polypeptide comprising a first light chain variable region (VL1); (c) a third polypeptide comprising from N-terminus to C-terminus: a second heavy-chain antibody variable domain (VHH2), a second linker peptide sequence, a second heavy chain variable region (VH2), a second hinge region, and a second Fc region; and (d) a fourth polypeptide comprising a second light chain variable region (VL2). In some embodiments, the VHH1 and the VHH2 specifically bind to BCMA. In some embodiments, the VH1 and the VL1 associate with each other and specifically bind to CD3. In some embodiments, the VH2 and the VL2 associate with each other and specifically bind to CD3.

In some embodiments, the first linker peptide sequence and/or the second linker peptide sequence comprises a sequence that is at least 80%, 85%, 90%, 95%, or 100% identical to any one SEQ ID NOs: 71-73 and 124-126. In some embodiments, the first linker peptide sequence and/or the second linker peptide sequence comprises a sequence that is at least 80% identical to SEQ ID NO: 72 or 73. In some embodiments, the first and/or the second linker peptide sequence comprise a sequence that is at least 80%, 85%, 90%, 95%, or 100% identical to one or more (e.g., 1, 2, 3, 4, 5, 6, 7, or 8) repeats of GGGGS (SEQ ID NO: 71).

In some embodiments, the first hinge region and/or the second hinge regions comprise a sequence that is at least 80%, 85%, 90%, 95%, or 100% identical to any one of SEQ ID NOs: 61-65.

In some embodiments, sequences of the VHH1 and the VHH2 are identical. In some embodiments, the first Fc region and/or the second Fc region comprise a sequence that is at least 80%, 85%, 90%, 95%, or 100% identical to SEQ ID NO: 68. In some embodiments, the first polypeptide comprises a sequence that is at least 80%, 85%, 90%, 95%, or 100% identical to SEQ ID NO: 108 or 109. In some embodiments, the second polypeptide comprises a sequence that is at least 80%, 85%, 90%, 95%, or 100% identical to SEQ ID NO: 110. In some embodiments, the third polypeptide comprises a sequence that is at least 80%, 85%, 90%, 95%, or 100% identical to SEQ ID NO: 108 or 109. In some embodiments, the fourth polypeptide comprises a sequence that is at least 80%, 85%, 90%, 95%, or 100% identical to SEQ ID NO: 110.

As shown in FIG. 8B, a BCMA/CD3 trispecific antibody can be prepared to have a TriSpecific-V4 structure. In some embodiments, sequences of the VHH1 and the VHH2 as described in the BiSpecific-V4 structure are different. In some embodiments, the first Fc region and/or the second Fc region comprise a sequence that is at least 80%, 85%, 90%, 95%, or 100% identical to SEQ ID NO: 66 or 67. In some embodiments, the first polypeptide comprises a sequence that is at least 80%, 85%, 90%, 95%, or 100% identical to SEQ ID NO: 111 or 112. In some embodiments, the second polypeptide comprises a sequence that is at least 80%, 85%, 90%, 95%, or 100% identical to SEQ ID NO: 115. In some embodiments, the third polypeptide comprises a sequence that is at least 80%, 85%, 90%, 95%, or 100% identical to SEQ ID NO: 113 or 114. In some embodiments, the fourth polypeptide comprises a sequence that is at least 80%, 85%, 90%, 95%, or 100% identical to SEQ ID NO: 115.

In some embodiments, the Fc region is an IgG1 (e.g., human IgG1) Fc region. In some embodiments, the BCMA/CD3 bispecific or trispecific antibody described herein comprises knob-into-hole mutations.

In some embodiments, the VHH that specifically binds to BCMA of the BCMA/CD3 bispecific antibody with the BiSpecific-V0, BiSpecific-V1, BiSpecific-V2, BiSpecific-V3, or BiSpecific-V4 structure can be selected from any of the VHHs targeting BCMA described in the disclosure. In some embodiments, the VHH that specifically binds to BCMA of the BCMA/CD3 trispecific antibody with the TriSpecific-V0, TriSpecific-V1, TriSpecific-V2, TriSpecific-V3, or TriSpecific-V4 structure can be selected from any of the VHHs targeting BCMA described in the disclosure.

In some embodiments, the VHH that specifically binds to CD3 of the BCMA/CD3 bispecific antibody with the BiSpecific-V0, BiSpecific-V1, BiSpecific-V2, BiSpecific-V3, or BiSpecific-V4 structure can be selected from any of the VHHs targeting CD3 described in the disclosure. In some embodiments, the VHH that specifically binds to CD3 of the BCMA/CD3 trispecific antibody with the TriSpecific-V0, TriSpecific-V1, TriSpecific-V2, TriSpecific-V3, or TriSpecific-V4 structure can be selected from any of the VHHs targeting CD3 described in the disclosure.

In some embodiments, the VH, when associated with a VL, that specifically binds to CD3 of the BCMA/CD3 bispecific antibody with the BiSpecific-V0, BiSpecific-V1, BiSpecific-V2, BiSpecific-V3, or BiSpecific-V4 structure can be selected from any of the VH targeting CD3 described in the disclosure. In some embodiments, the VH, when associated with a VL, that specifically binds to CD3 of the BCMA/CD3 trispecific antibody with the TriSpecific-V0, TriSpecific-V1, TriSpecific-V2, TriSpecific-V3, or TriSpecific-V4 structure can be selected from any of the VH targeting CD3 described in the disclosure.

In some embodiments, the VL, when associated with a VH, that specifically binds to CD3 of the BCMA/CD3 bispecific antibody with the BiSpecific-V0, BiSpecific-V1, BiSpecific-V2, BiSpecific-V3, or BiSpecific-V4 structure can be selected from any of the VL targeting CD3 described in the disclosure. In some embodiments, the VL, when associated with a VH, that specifically binds to CD3 of the BCMA/CD3 trispecific antibody with the TriSpecific-V0, TriSpecific-V1, TriSpecific-V2, TriSpecific-V3, or TriSpecific-V4 structure can be selected from any of the VL targeting CD3 described in the disclosure.

### (2) CD38 CD3 Multispecific Antibody

CD38 (cluster of differentiation 38, also known as cyclic ADP ribose hydrolase) is a glycoprotein found on the surface of many immune cells (white blood cells), including CD4+, CD8+, B lymphocytes and natural killer cells. CD38 also functions in cell adhesion, signal transduction and calcium signaling. It is a cancer antigen and is often expressed on e.g., blood cancer cells. The present disclosure provides bispecific antibodies that bind to both CD38 and CD3. The multispecific (e.g., bispecific or trispecific) antibodies can be used to treat CD38 positive cancers (e.g., multiple myeloma or hematological malignancy) in a subject.

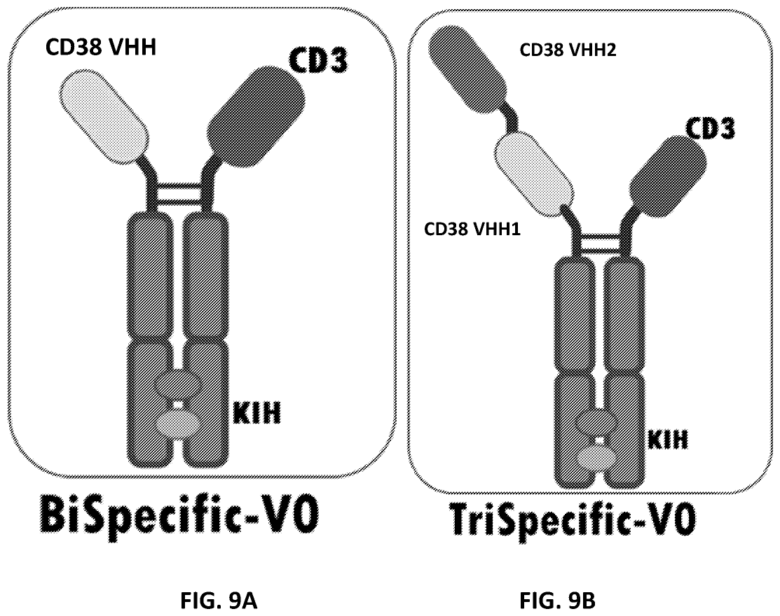

As shown in FIG. 9A, a CD38/CD3 bispecific antibody can be prepared to have a BiSpecific-V0 structure. Specifically, the CD38/CD3 bispecific antibody comprises (a) a first polypeptide comprising from N-terminus to C-terminus: a first heavy-chain antibody variable domain (VHH), a first hinge region, a first Fc region; and (b) a second polypeptide comprising from N-terminus to C-terminus: a second VHH, a second hinge region, and a second Fc region. In some embodiments, the first VHH specifically binds to CD38, and the second VHH specifically binds to CD3.

In some embodiments, the first hinge region and/or the second hinge region comprise a sequence that is at least 80%, 85%, 90%, 95%, or 100% identical to any one of SEQ ID NOs: 61-65. In some embodiments, the first Fc region and/or the second Fc region comprise a sequence that is at least 80%, 85%, 90%, 95%, or 100% identical to SEQ ID NO: 66 or 67.

In some embodiments, the CD38/CD3 bispecific antibody comprises knob-into-hole mutations. In some embodiments, the Fc region is an IgG1 Fc region. In some embodiments, the first polypeptide comprises a sequence that is at least 80%, 85%, 90%, 95%, or 100% identical to SEQ ID NO: 116 or 117. In some embodiments, the second polypeptide comprises a sequence that is at least 80% identical to SEQ ID NOs: 118 or 119.

As shown in FIG. 9B, a CD38/CD3 trispecific antibody can be prepared to have a TriSpecific-V0 structure. Specifically, the first polypeptide as described in the BiSpecific-V0 structure can further include a third VHH that specifically binds to CD38, wherein the third VHH is linked to the N-terminus of the first VHH via a linker peptide sequence. In some embodiments, the linker peptide sequence comprises a sequence that is at least 80%, 85%, 90%, 95%, or 100% identical to any one SEQ ID NOs: 71-73 and 124-126. In some embodiments, the linker peptide sequence comprises a sequence that is at least 80%, 85%, 90%, 95%, or 100% identical to SEQ ID NO: 72 or 73. In some embodiments, the linker peptide sequence comprises a sequence that is at least 80%, 85%, 90%, 95%, or 100% identical to one or more (e.g., 1, 2, 3, 4, 5, 6, 7, or 8) repeats of GGGGS (SEQ ID NO: 71).

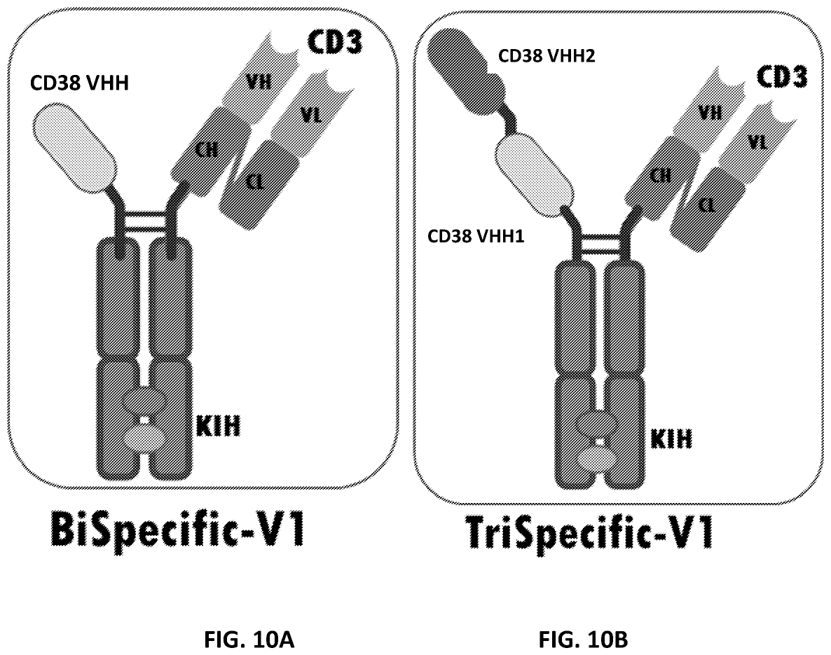

As shown in FIG. 10A, a CD38/CD3 bispecific antibody can be prepared to have a BiSpecific-V1 structure. Specifically, the CD38/CD3 bispecific antibody comprises (a) a first polypeptide comprising from N-terminus to C-terminus: a first heavy-chain antibody variable domain (VHH), a first hinge region, a first Fc region; (b) a second polypeptide comprising from N-terminus to C-terminus: a heavy chain variable region (VH), a second hinge region, and a second Fc region; and (c) a third polypeptide comprising a light chain variable region (VL). In some embodiments, the first VHH specifically binds to CD38, wherein the VH and the VL associate with each other and specifically bind to CD3.

In some embodiments, the first hinge region and/or the second hinge region comprise a sequence that is at least 80%, 85%, 90%, 95%, or 100% identical to any one of SEQ ID NOs: 61-65. In some embodiments, the first Fc region and/or the second Fc region comprise a sequence that is at least 80%, 85%, 90%, 95%, or 100% identical to SEQ ID NO: 66 or 67.

In some embodiments, the CD38/CD3 bispecific antibody comprises knob-into-hole mutations. In some embodiments, the Fc region is an IgG1 Fc region.

As shown in FIG. 10B, a CD38/CD3 trispecific antibody can be prepared to have a TriSpecific-V1 structure. Specifically, the first polypeptide as described in the BiSpecific-V1 structure can further include a second VHH that specifically binds to CD38, wherein the second VHH is linked to the N-terminus of the first VHH via a linker peptide sequence. In some embodiments, the linker peptide sequence comprises a sequence that is at least 80%, 85%, 90%, 95%, or 100% identical to any one SEQ ID NOs: 71-73 and 124-126. In some embodiments, the linker peptide sequence comprises a sequence that is at least 80% identical to SEQ ID NO: 72 or 73. In some embodiments, the linker peptide sequence comprises a sequence that is at least 80%, 85%, 90%, 95%, or 100% identical to one or more (e.g., 1, 2, 3, 4, 5, 6, 7, or 8) repeats of GGGGS (SEQ ID NO: 71).

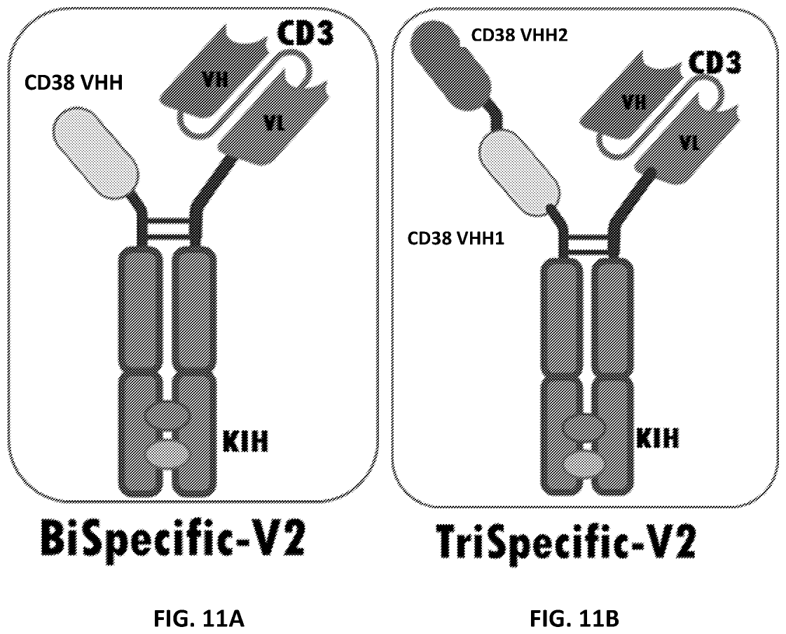

As shown in FIG. 11A, a CD38/CD3 bispecific antibody can be prepared to have a BiSpecific-V2 structure. Specifically, the CD38/CD3 bispecific antibody comprises (a) a first polypeptide comprising from N-terminus to C-terminus: a first heavy-chain antibody variable domain (VHH), a first hinge region, a first Fc region; and (b) a second polypeptide comprising from N-terminus to C-terminus: a single-chain variable fragment (scFv), a second hinge region, and a second Fc region.

In some embodiments, the first VHH specifically binds to CD38. In some embodiments, the scFv comprises a heavy chain variable region (VH), a first linker peptide sequence, and a light chain variable region (VL). In some embodiments, the VH and the VL associate with each other and specifically bind to CD3.

In some embodiments, the first linker peptide sequence comprises a sequence that is at least 80%, 85%, 90%, 95%, or 100% identical to any one SEQ ID NOs: 71-73 and 124-126. In some embodiments, the first linker peptide sequence comprises a sequence that is at least 80%, 85%, 90%, 95%, or 100% identical to SEQ ID NO: 72 or 73. In some embodiments, the first hinge region and/or the second hinge region comprise a sequence that is at least 80%, 85%, 90%, 95%, or 100% identical to any one of SEQ ID NOs: 61-65. In some embodiments, the first Fc region and/or the second Fc region comprise a sequence that is at least 80%, 85%, 90%, 95%, or 100% identical to SEQ ID NO: 66 or 67.

In some embodiments, the CD38/CD3 bispecific antibody comprises knob-into-hole mutations. In some embodiments, the Fc region is an IgG1 Fc region. In some embodiments, the first polypeptide comprises a sequence that is at least 80%, 85%, 90%, 95%, or 100% identical to SEQ ID NO: 120 or 121; wherein the second polypeptide comprises a sequence that is at least 80%, 85%, 90%, 95%, or 100% identical to SEQ ID NO: 122 or 123.

As shown in FIG. 11B, a CD38/CD3 trispecific antibody can be prepared to have a TriSpecific-V2 structure. Specifically, the first polypeptide as described in the BiSpecific-V2 structure can further include a second VHH that specifically binds to CD38, wherein the second VHH is linked to the N-terminus of the first VHH via a second linker peptide sequence.

In some embodiments, the second linker peptide sequence comprises a sequence that is at least 80%, 85%, 90%, 95%, or 100% identical to any one SEQ ID NOs: 71-73 and 124-126. In some embodiments, the second linker peptide sequence comprises a sequence that is at least 80%, 85%, 90%, 95%, or 100% identical to SEQ ID NO: 72 or 73. In some embodiments, the first and/or the second linker peptide sequence comprise a sequence that is at least 80%, 85%, 90%, 95%, or 100% identical to one or more (e.g., 1, 2, 3, 4, 5, 6, 7, or 8) repeats of GGGGS (SEQ ID NO: 71).

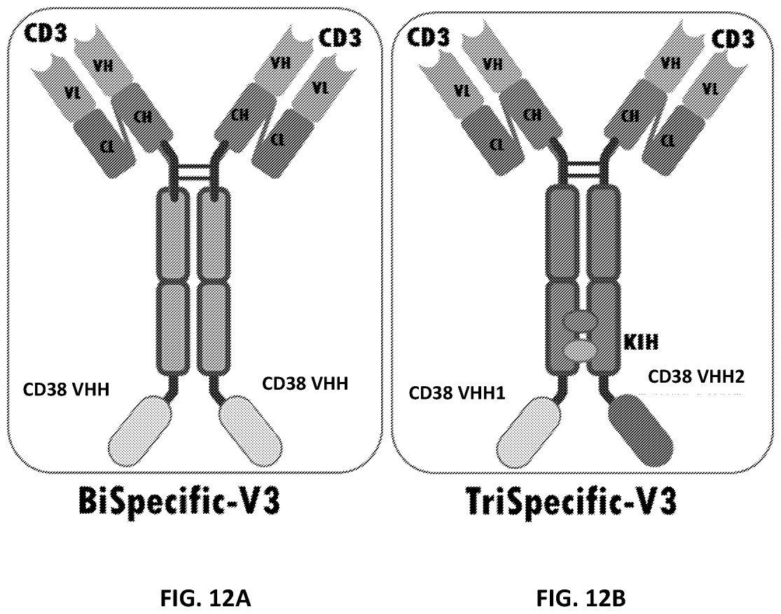

As shown in FIG. 12A, a CD38/CD3 bispecific antibody can be prepared to have a BiSpecific-V3 structure. Specifically, the CD38/CD3 bispecific antibody comprises (a) a first polypeptide comprising from N-terminus to C-terminus: a first heavy chain variable region (VH1), a first hinge region, a first Fc region; a first linker peptide sequence, and a first heavy-chain antibody variable domain (VHH1); (b) a second polypeptide comprising a first light chain variable region (VL1); (c) a third polypeptide comprising from N-terminus to C-terminus: a second heavy chain variable region (VH2), a second hinge region, a second Fc region, a second linker peptide sequence, and a second heavy-chain antibody variable domain (VHH2); and (d) a fourth polypeptide comprising a second light chain variable region (VL2). In some embodiments, the VHH1 and/or the VHH2 specifically bind to CD38. In some embodiments, the VH1 and the VL1 associate with each other and specifically bind to CD3. In some embodiments, the VH2 and the VL2 associate with each other and specifically bind to CD3.

In some embodiments, the first linker peptide sequence and/or the second linker peptide sequence comprises a sequence that is at least 80%, 85%, 90%, 95%, or 100% identical to any one SEQ ID NOs: 71-73 and 124-126. In some embodiments, the first linker peptide sequence and/or the second linker peptide sequence comprises a sequence that is at least 80%, 85%, 90%, 95%, or 100% identical to SEQ ID NO: 72 or 73. In some embodiments, the first and/or the second linker peptide sequence comprise a sequence that is at least 80%, 85%, 90%, 95%, or 100% identical to one or more (e.g., 1, 2, 3, 4, 5, 6, 7, or 8) repeats of GGGGS (SEQ ID NO: 71).

In some embodiments, the first hinge region and/or the second hinge regions comprise a sequence that is at least 80%, 85%, 90%, 95%, or 100% identical to any one of SEQ ID NOs: 61-65.

In some embodiments, sequences of the VHH1 and the VHH2 are identical. In some embodiments, the first Fc region and/or the second Fc region comprise a sequence that is at least 80%, 85%, 90%, 95%, or 100% identical to SEQ ID NO: 68.

As shown in FIG. 12B, a CD38/CD3 trispecific antibody can be prepared to have a TriSpecific-V3 structure. In some embodiments, sequences of the VHH1 and the VHH2 as described in the BiSpecific-V3 structure are different. In some embodiments, the first Fc region and/or the second Fc region comprise a sequence that is at least 80%, 85%, 90%, 95%, or 100% identical to SEQ ID NO: 66 or 67.

In some embodiments, the Fc region is an IgG1 (e.g., human IgG1) Fc region. In some embodiments, the CD38/CD3 bispecific or trispecific antibody described herein comprises knob-into-hole mutations.

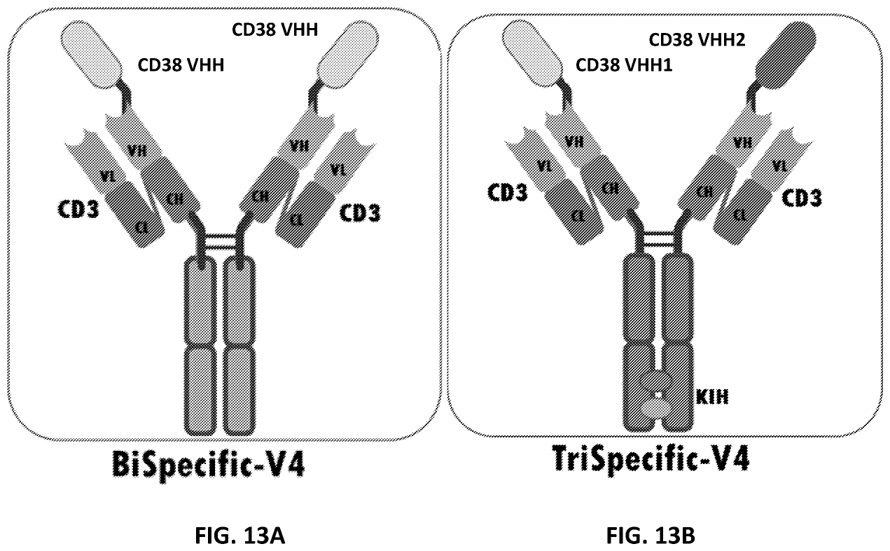

As shown in FIG. 13A, a CD38/CD3 bispecific antibody can be prepared to have a BiSpecific-V4 structure. Specifically, the CD38/CD3 bispecific antibody comprises (a) a first polypeptide comprising from N-terminus to C-terminus: a first heavy-chain antibody variable domain (VHH1), a first linker peptide sequence, a first heavy chain variable region (VH1), a first hinge region, and a first Fc region; (b) a second polypeptide comprising a first light chain variable region (VL1); (c) a third polypeptide comprising from N-terminus to C-terminus: a second heavy-chain antibody variable domain (VHH2), a second linker peptide sequence, a second heavy chain variable region (VH2), a second hinge region, and a second Fc region; and (d) a fourth polypeptide comprising a second light chain variable region (VL2). In some embodiments, the VHH1 and the VHH2 specifically bind to CD38. In some embodiments, the VH1 and the VL1 associate with each other and specifically bind to CD3. In some embodiments, the VH2 and the VL2 associate with each other and specifically bind to CD3.

In some embodiments, the first linker peptide sequence and/or the second linker peptide sequence comprises a sequence that is at least 80%, 85%, 90%, 95%, or 100% identical to any one SEQ ID NOs: 71-73 and 124-126. In some embodiments, the first linker peptide sequence and/or the second linker peptide sequence comprises a sequence that is at least 80% identical to SEQ ID NO: 72 or 73. In some embodiments, the first and/or the second linker peptide sequence comprise a sequence that is at least 80%, 85%, 90%, 95%, or 100% identical to one or more (e.g., 1, 2, 3, 4, 5, 6, 7, or 8) repeats of GGGGS (SEQ ID NO: 71).

In some embodiments, the first hinge region and/or the second hinge regions comprise a sequence that is at least 80%, 85%, 90%, 95%, or 100% identical to any one of SEQ ID NOs: 61-65.

In some embodiments, sequences of the VHH1 and the VHH2 are identical. In some embodiments, the first Fc region and/or the second Fc region comprise a sequence that is at least 80%, 85%, 90%, 95%, or 100% identical to SEQ ID NO: 68.

As shown in FIG. 13B, a CD38/CD3 trispecific antibody can be prepared to have a TriSpecific-V4 structure. In some embodiments, sequences of the VHH1 and the VHH2 as described in the BiSpecific-V4 structure are different. In some embodiments, the first Fc region and/or the second Fc region comprise a sequence that is at least 80%, 85%, 90%, 95%, or 100% identical to SEQ ID NO: 66 or 67.

In some embodiments, the Fc region is an IgG1 (e.g., human IgG1) Fc region. In some embodiments, the CD38/CD3 bispecific or trispecific antibody described herein comprises knob-into-hole mutations.

In some embodiments, the VHH that specifically binds to CD38 of the CD38/CD3 bispecific antibody with the BiSpecific-V0, BiSpecific-V1, BiSpecific-V2, BiSpecific-V3, or BiSpecific-V4 structure can be selected from any of the VHHs targeting CD38 described in the disclosure. In some embodiments, the VHH that specifically binds to CD38 of the CD38/CD3 trispecific antibody with the TriSpecific-V0, TriSpecific-V1, TriSpecific-V2, TriSpecific-V3, or TriSpecific-V4 structure can be selected from any of the VHHs targeting CD38 described in the disclosure.

In some embodiments, the VHH that specifically binds to CD3 of the CD38/CD3 bispecific antibody with the BiSpecific-V0, BiSpecific-V1, BiSpecific-V2, BiSpecific-V3, or BiSpecific-V4 structure can be selected from any of the VHHs targeting CD3 described in the disclosure. In some embodiments, the VHH that specifically binds to CD3 of the CD38/CD3 trispecific antibody with the TriSpecific-V0, TriSpecific-V1, TriSpecific-V2, TriSpecific-V3, or TriSpecific-V4 structure can be selected from any of the VHHs targeting CD3 described in the disclosure.

In some embodiments, the VH, when associated with a VL, that specifically binds to CD3 of the CD38/CD3 bispecific antibody with the BiSpecific-V0, BiSpecific-V1, BiSpecific-V2, BiSpecific-V3, or BiSpecific-V4 structure can be selected from any of the VH targeting CD3 described in the disclosure. In some embodiments, the VH, when associated with a VL, that specifically binds to CD3 of the CD38/CD3 trispecific antibody with the TriSpecific-V0, TriSpecific-V1, TriSpecific-V2, TriSpecific-V3, or TriSpecific-V4 structure can be selected from any of the VH targeting CD3 described in the disclosure.

In some embodiments, the VL, when associated with a VH, that specifically binds to CD3 of the CD38/CD3 bispecific antibody with the BiSpecific-V0, BiSpecific-V1, BiSpecific-V2, BiSpecific-V3, or BiSpecific-V4 structure can be selected from any of the VL targeting CD3 described in the disclosure. In some embodiments, the VL, when associated with a VH, that specifically binds to CD3 of the CD38/CD3 trispecific antibody with the TriSpecific-V0, TriSpecific-V1, TriSpecific-V2, TriSpecific-V3, or TriSpecific-V4 structure can be selected from any of the VL targeting CD3 described in the disclosure.

### (3) BCMA CD38 CD3 Multispecific Antibody, and BCMA CD38 Multispecific Antibody

In some embodiments, the disclosure is related to a multispecific (e.g., tri-specific) antibody or antigen binding fragment thereof that specifically binds to BCMA, CD38 and CD3. The multispecific (e.g., trispecific) antibodies can be used to treat BCMA and/or CD38 positive cancers (e.g., multiple myeloma or hematological malignancy) in a subject. In some embodiments, the multispecific antibodies described herein targeting both BCMA and CD38 can reduce the chance of immune escape of cancer cells. In some embodiments, the cancer cells express BCMA but not CD38, the cancer cells express CD38 but not BCMA, or the cancer cells express both BCMA and CD38.

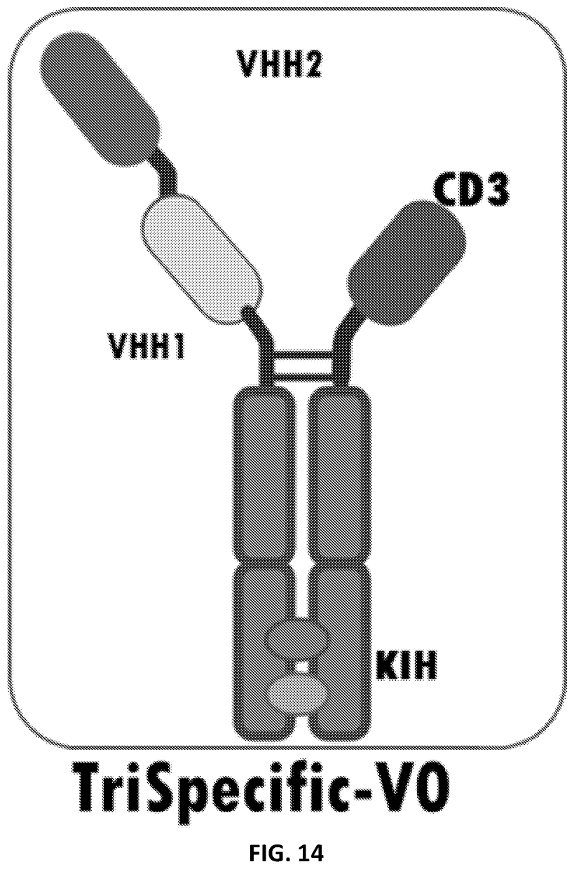

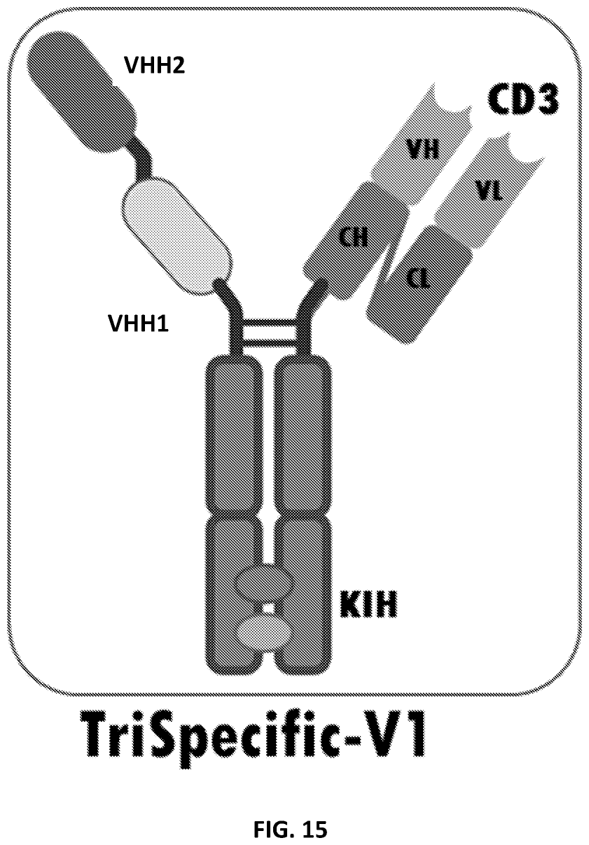

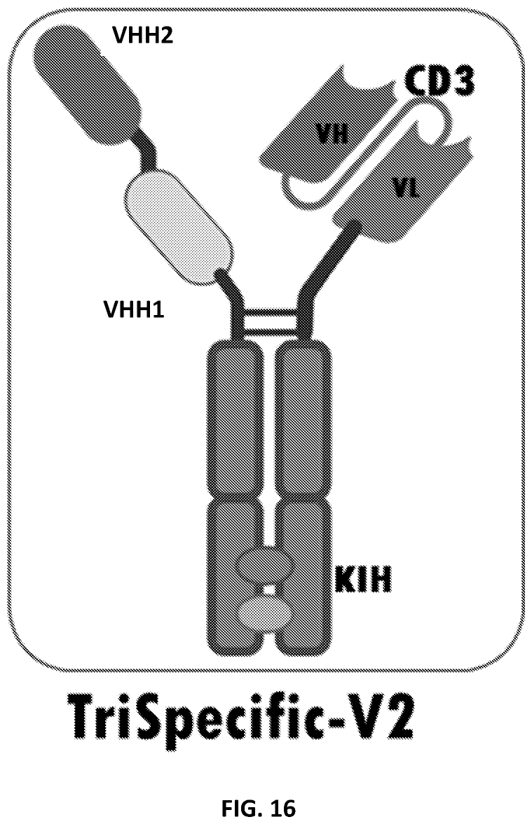

As shown in FIGS. 14-16, and 17A-17B, a BCMA/CD38/CD3 trispecific antibody can be prepared to have a TriSpecific-V0, TriSpecific-V1, TriSpecific-V2, TriSpecific-V3, or TriSpecific-V4 structure. For example, the BCMA/CD38/CD3 trispecific antibody can have a first heavy-chain antibody variable domain (VHH1) that specifically binds to BCMA, and a second heavy-chain antibody variable domain (VHH2) that specifically binds to CD38. Alternatively, the BCMA/CD38/CD3 trispecific antibody can have a VHH1 that specifically binds to CD38, and a VHH2 that specifically binds to BCMA. In some embodiments, the VHH that specifically binds to BCMA of the BCMA/CD38/CD3 trispecific antibody with the TriSpecific-V0, TriSpecific-V1, TriSpecific-V2, TriSpecific-V3, or TriSpecific-V4 structure can be selected from any of the VHHs targeting BCMA described in the disclosure. In some embodiments, the VHH that specifically binds to CD38 of the BCMA/CD38/CD3 trispecific antibody with the TriSpecific-V0, TriSpecific-V1, TriSpecific-V2, TriSpecific-V3, or TriSpecific-V4 structure can be selected from any of the VHHs targeting CD38 described in the disclosure. In some embodiments, the VHH that specifically binds to CD3 of the BCMA/CD38/CD3 trispecific antibody with the TriSpecific-V0, TriSpecific-V1, TriSpecific-V2, TriSpecific-V3, or TriSpecific-V4 structure can be selected from any of the VHHs targeting CD3 described in the disclosure.

In some embodiments, the VH, when associated with a VL, that specifically binds to CD3 of the BCMA/CD38/CD3 trispecific antibody with the TriSpecific-V0, TriSpecific-V1, TriSpecific-V2, TriSpecific-V3, or TriSpecific-V4 structure can be selected from any of the VH targeting CD3 described in the disclosure. In some embodiments, the VL, when associated with a VH, that specifically binds to CD3 of the BCMA/CD38/CD3 trispecific antibody with the BiSpecific-V0, BiSpecific-V1, BiSpecific-V2, BiSpecific-V3, or BiSpecific-V4 structure can be selected from any of the VL targeting CD3 described in the disclosure.

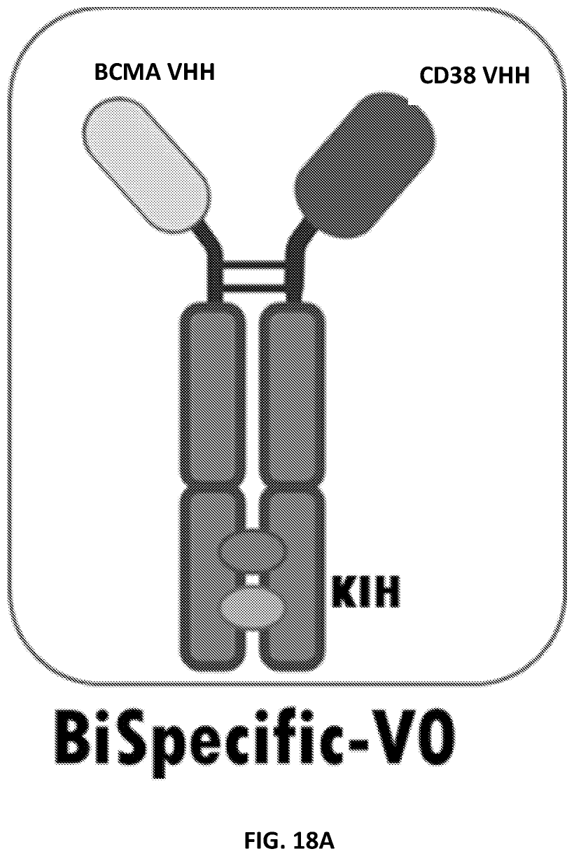

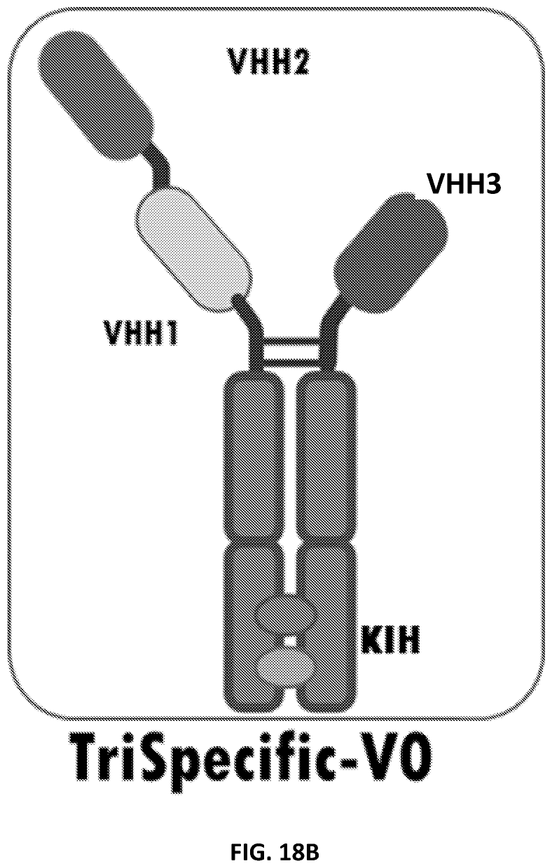

As shown in FIG. 18A, a BCMA/CD38 bispecific antibody can be prepared to have a BiSpecific-V0 structure. In some embodiments, the VHH that specifically binds to BCMA of the BCMA/CD38 bispecific antibody with the BiSpecific-V0 structure can be selected from any of the VHHs targeting BCMA described in the disclosure. In some embodiments, the VHH that specifically binds to CD38 of the BCMA/CD38 bispecific antibody with the BiSpecific-V0 structure can be selected from any of the VHHs targeting CD38 described in the disclosure.

As shown in FIG. 18B, a BCMA/CD38 multispecific (e.g., bispecific or trispecific) antibody can be prepared to have a TriSpecific-V0 structure. In some embodiments, the BCMA/CD38 multispecific antibody can have a first heavy-chain antibody variable domain (VHH1) that specifically binds to BCMA or CD38. In some embodiments, the BCMA/CD38 multispecific antibody can have a second heavy-chain antibody variable domain (VHH2) that specifically binds to BCMA or CD38. In some embodiments, the BCMA/CD38 multispecific antibody can have a third heavy-chain antibody variable domain (VHH3) that specifically binds to BCMA or CD38. In some embodiments, the VHH1, VHH2, or VHH3 that specifically binds to BCMA of the BCMA/CD38 multispecific antibody with the TriSpecific-V0 structure can be selected from any of the VHHs targeting BCMA described in the disclosure. In some embodiments, the VHH1, VHH2, or VHH3 that specifically binds to CD38 of the BCMA/CD38 multispecific antibody with the TriSpecific-V0 structure can be selected from any of the VHHs targeting CD38 described in the disclosure.

### Antibody Characteristics

The anti-BCMA, anti-CD38, anti-CD3, anti-BCMA/CD3, anti-CD38/CD3, or anti-BCMA/CD38/CD3 antigen-binding protein construct (e.g., antibodies, bispecific antibodies, trispecific antibodies, multi-specific antibodies, or antibody fragments thereof) can include an antigen binding site that is derived from any anti-BCMA antibody, anti-CD38 antibody, anti-CD3 antibody, or any antigen-binding fragment thereof as described herein.

In some embodiments, the antibodies, or antigen-binding fragments thereof described herein can bind to BCMA and CD3, thereby bridging the interaction between cancer cell (e.g., BCMA-expressing cancer cell) and T cell. In some embodiments, the antibodies, or antigen-binding fragments thereof described herein can bind to CD38 and CD3, thereby bridging the interaction between cancer cell (e.g., CD38-expressing cancer cell) and T cell.

In some embodiments, the antibodies, or antigen-binding fragments thereof described herein can bind to BCMA-expressing cells (e.g., Raji cells). In some embodiments, at least or about 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30% of the BCMA-expressing cells (e.g., Raji cells) are bound to the antibodies, or antigen-binding fragments thereof described herein after incubation at room temperature for at least or about 10 minutes, 20 minutes, 30 minutes, 40 minutes, 50 minutes, 1 hour, 2 hours, 3 hours, 4 hours, or 5 hours.

In some embodiments, the antibodies, or antigen-binding fragments thereof described herein can bind to CD3-expressing cells (e.g., Jurkat cells). In some embodiments, at least or about 0.2%, 0.4%, 0.6%, 0.8%, 1%, 1.2%, 1.4%, 1.6%, 1.8%, 2%, 2.2%, 2.4%, 2.6%, 2.8%, 3%, 3.2%, 3.4%, 3.6%, 3.8%, 4%, 4.2%, 4.4%, 4.6%, 4.8%, or 5% of the CD3-expressing cells (e.g., Jurkat cells) are bound to the antibodies, or antigen-binding fragments thereof described herein after incubation at room temperature for at least or about 10 minutes, 20 minutes, 30 minutes, 40 minutes, 50 minutes, 1 hour, 2 hours, 3 hours, 4 hours, or 5 hours.

In some embodiments, the antibodies, or antigen-binding fragments thereof described herein can induce T cell activation. In some embodiments, the T cell activation level induced by the antibodies, or antigen-binding fragments thereof described herein is at least or about 1-fold, 2-fold, 3-fold, 4-fold, 5-fold, 6-fold, 7-fold, 8-fold, 9-fold, 10-fold, 20-fold, 50-fold, or 100-fold as compared to that induced by an isotype control antibody.

The antibodies or antigen-binding fragments thereof (e.g., bispecific antibodies) as described herein can increase immune response. In some embodiments, the antibodies or antigen-binding fragments thereof as described herein can increase immune response, activity or number of T cells (e.g., CD3+ cells, CD8+ and/or CD4+ cells) by at least 10%, 20%, 30%, 40%, 50%, 60%, 70%, 80%, 90%, 100%, 2 folds, 3 folds, 5 folds, 10 folds, or 20 folds.

In some embodiments, the antibodies or antigen-binding fragments thereof as described herein does not induce immune response in normal cells (e.g., non-tumor cells) or in the absence of tumor cells.

In some embodiments, the antibodies or antigen-binding fragments thereof as described herein are CD3 antagonist. In some embodiments, the antibodies or antigen-binding fragments thereof are CD3 agonist.

In some embodiments, the antibodies or antigen-binding fragments thereof (e.g., bispecific antibodies) can bind to CD3. Thus, the antibodies or antigen-binding fragments thereof described herein can recruit T cells to a target cell.

In some embodiments, the antibody (or antigen-binding fragments thereof) specifically binds to an antigen (e.g., a cancer antigen) with a dissociation rate (koff) of less than 0.1 s⁻¹, less than 0.01 s⁻¹, less than 0.001 s⁻¹, less than 0.0001 s⁻¹, or less than 0.00001 s⁻¹. In some embodiments, the dissociation rate (koff) is greater than 0.01 s⁻¹, greater than 0.001 s⁻¹, greater than 0.0001 s⁻¹, greater than 0.00001 s⁻¹, or greater than 0.000001 s⁻¹. In some embodiments, kinetic association rates (kon) is greater than 1×10²/Ms, greater than 1×10³/Ms, greater than 1×10⁴/Ms, greater than 1×10⁵/Ms, or greater than 1×10⁶/Ms. In some embodiments, kinetic association rates (kon) is less than 1×10⁵/Ms, less than 1×10⁶/Ms, or less than 1×10⁷/Ms.

Affinities can be deduced from the quotient of the kinetic rate constants (Kd=koff/kon). In some embodiments, Kd is less than 1×10⁻⁴M, less than 1×10⁻⁵ M, less than 1×10⁻⁶ M, less than 1×10⁻⁷M, less than 1×10⁻⁸ M, less than 1×10⁻⁹ M, or less than 1×10⁻¹⁰ M. In some embodiments, the Kd is less than 50 nM, 30 nM, 20 nM, 15 nM, 10 nM, 9 nM, 8 nM, 7 nM, 6 nM, 5 nM, 4 nM, 3 nM, 2 nM, or 1 nM. In some embodiments, Kd is greater than 1×10⁻⁴M, greater than 1×10⁻⁵ M, greater than 1×10⁻⁶ M, greater than 1×10⁻⁷ M, greater than 1×10⁻⁸ M, greater than 1×10⁻⁹ M, greater than 1×10⁻¹⁰ M, greater than 1×10⁻¹¹ M, or greater than 1×10⁻¹² M. Furthermore, Ka can be deduced from Kd by the formula Ka=1/Kd.

In some embodiments, the binding affinity to CD3 is carefully adjusted, e.g., Kd can be between 1000 nM˜10 nM, between 1000 nM˜50 nM, between 1000 nM˜100 nM, between 500 nM˜10 nM, between 500 nM˜50 nM, or between 500 nM˜100 nM.

General techniques for measuring the affinity of an antibody for an antigen include, e.g., ELISA, RIA, and surface plasmon resonance (SPR).

In some embodiments, the antibody has a tumor growth inhibition percentage (TGI %) that is greater than 10%, 20%, 30%, 40%, 50%, 60%, 70%, 80%, 90%, 100%, 110%, 120%, 130%, 140%, 150%, 160%, 170%, 180%, 190%, or 200%. In some embodiments, the antibody has a tumor growth inhibition percentage that is less than 60%, 70%, 80%, 90%, 100%, 110%, 120%, 130%, 140%, 150%, 160%, 170%, 180%, 190%, or 200%. The TGI % can be determined, e.g., at 3, 4, 5, 6, 7, 8, 9, 10, 11, 12, 13, 14, 15, 16, 17, 18, 19, 20, 21, 22, 23, 24, 25, 26, 27, 28, 29, or 30 days after the treatment starts, or 1, 2, 3, 4, 5, 6, 7, 8, 9, 10, 11, or 12 months after the treatment starts. As used herein, the tumor growth inhibition percentage (TGI %) is calculated using the following formula:

TGI (%)=[1−(Ti−T0)/(Vi−V0)]×100

Ti is the average tumor volume in the treatment group on day i. T0 is the average tumor volume in the treatment group on day zero. Vi is the average tumor volume in the control group on day i. V0 is the average tumor volume in the control group on day zero.

In some embodiments, the antibodies or antigen binding fragments can increase complement dependent cytotoxicity (CDC) by at least 10%, 20%, 30%, 40%, 50%, 60%, 70%, 80%, 90%, 100%, 2 folds, 3 folds, 5 folds, 10 folds, or 20 folds.

In some embodiments, the antibodies or antigen binding fragments can increase antibody-dependent cell-mediated cytotoxicity (ADCC) by at least 10%, 20%, 30%, 40%, 50%, 60%, 70%, 80%, 90%, 100%, 2 folds, 3 folds, 5 folds, 10 folds, or 20 folds.

In some embodiments, the antibodies or antigen binding fragments can increase internalization rate by at least 10%, 20%, 30%, 40%, 50%, 60%, 70%, 80%, 90%, 100%, 2 folds, 3 folds, 5 folds, 10 folds, or 20 folds.

In some embodiments, the antibodies or antigen binding fragments can increase phagocytosis rate by at least 10%, 20%, 30%, 40%, 50%, 60%, 70%, 80%, 90%, 100%, 2 folds, 3 folds, 5 folds, 10 folds, or 20 folds.

In some embodiments, the antibodies or antigen binding fragments can enhance T cell function, for example, by increasing effector T cell proliferation and/or increasing gamma interferon production by the effector T cell (e.g., as compared to proliferation and/or cytokine production prior to treatment with the antibodies or antigen binding fragments).

In some embodiments, the antibodies or antigen binding fragments enhance CD4+ effector T cell function, for example, by increasing CD4+ effector T cell proliferation and/or increasing gamma interferon production by the CD4+ effector T cell (e.g., as compared to proliferation and/or cytokine production prior to treatment with the antibodies or antigen binding fragments). In some embodiments, the cytokine is gamma interferon. In some embodiments, the antibodies or antigen binding fragments increase number of intratumoral (infiltrating) CD4+ effector T cells (e.g., total number of CD4+ effector T cells, or e.g., percentage of CD4+ cells in CD45+ cells), e.g., as compared to number of intratumoral (infiltrating) CD4+ T cells prior to treatment with antibodies or antigen binding fragments. In some embodiments, the antibodies or antigen binding fragments increase number of intratumoral (infiltrating) CD4+ effector T cells that express gamma interferon (e.g., total gamma interferon expressing CD4+ cells, or e.g., percentage of gamma interferon expressing CD4+ cells in total CD4+ cells), e.g., as compared to number of intratumoral (infiltrating) CD4+ T cells that express gamma interferon prior to treatment.

In some embodiments, the antibodies or antigen binding fragments increase number of intratumoral (infiltrating) CD8+ effector T cells (e.g., total number of CD8+ effector T cells, or e.g., percentage of CD8+ in CD45+ cells), e.g., as compared to number of intratumoral (infiltrating) CD8+T effector cells prior to treatment. In some embodiments, the antibodies or antigen binding fragments increase number of intratumoral (infiltrating) CD8+ effector T cells that express gamma interferon (e.g., percentage of CD8+ cells that express gamma interferon in total CD8+ cells), e.g., compared to number of intratumoral (infiltrating) CD8+ T cells that express gamma interferon prior to treatment with the antibody.

In some embodiments, the antibodies or antigen binding fragments enhance memory T cell function, for example by increasing memory T cell proliferation and/or increasing cytokine (e.g., gamma interferon) production by the memory cell.

In some embodiments, the antibodies or antigen binding fragments have a functional Fc region. In some embodiments, effector function of a functional Fc region is antibody-dependent cell-mediated cytotoxicity (ADCC). In some embodiments, effector function of a functional Fc region is phagocytosis. In some embodiments, effector function of a functional Fc region is ADCC and phagocytosis. In some embodiments, the Fc region is human IgG1, human IgG2, human IgG3, or human IgG4.

In some embodiments, the antibodies or antigen binding fragments can induce apoptosis.

In some embodiments, the antibodies or antigen binding fragments do not have a functional Fc region. For example, the antibodies or antigen binding fragments are Fab, Fab′, F(ab′)2, and Fv fragments.

In some embodiments, the antibodies or antigen binding fragments are humanized antibodies. Humanization percentage means the percentage identity of the heavy chain or light chain variable region sequence as compared to human antibody sequences in International Immunogenetics Information System (IMGT) database. The top hit means that the heavy chain or light chain variable region sequence is closer to a particular species than to other species. For example, top hit to human means that the sequence is closer to human than to other species. Top hit to human and Macaca fascicularis means that the sequence has the same percentage identity to the human sequence and the Macaca fascicularis sequence, and these percentages identities are highest as compared to the sequences of other species. In some embodiments, humanization percentage is greater than 80%, 81%, 82%, 83%, 84%, 85%, 86%, 87%, 88%, 89%, 90%, 91%, 92%, 93%, 94%, or 95%. A detailed description regarding how to determine humanization percentage and how to determine top hits is known in the art, and is described, e.g., in Jones, et al. “The INNs and outs of antibody nonproprietary names.” MAbs. Vol. 8. No. 1. Taylor & Francis, 2016, which is incorporated herein by reference in its entirety. A high humanization percentage often has various advantages, e.g., more safe and more effective in humans, more likely to be tolerated by a human subject, and/or less likely to have side effects.

In some embodiments, the multi-specific antibody including the bispecific antibody described herein (e.g., a BCMA/CD3, or CD38/CD3 bispecific antibody) or the trispecific antibody described herein (e.g., a BCMA/CD38/CD3 trispecific antibody) has an asymmetric structure comprising: 2, 3, 4, 5, or 6 antigen binding sites. In some embodiments, the multi-specific antibody described herein comprises 2, 3, 4, 5, or 6 antigen binding sites (e.g., antigen binding Fab domains, scFV, or naonbody (VHH)) that target a cancer antigen (e.g., BCMA or CD38). In some embodiments, the multi-specific antibody described herein comprises 2, 3, 4, 5, or 6 antigen binding sites (e.g., antigen binding Fab domains, scFV, or naonbody (VHH)) that target a T cell specific antigen (e.g., CD3). In some embodiments, the multi-specific antibody described herein (e.g., a BCMA/CD3 bispecific antibody, a CD38/CD3 bispecific antibody, or a BCMA/CD38/CD3 trispecific antibody) comprises at least 2, 3, 4, 5, 6, or 7 common light chains. In some embodiments, the at least 2, 3, 4, 5, 6, or 7 common light chains have the same VL sequence. In some embodiments, the at least 2, 3, 4, 5, 6, or 7 common light chains have different VL sequences. In some embodiments, the cancer-specific antigen (e.g., BCMA or CD38) binding Fab domain comprises the same VH sequence. In some embodiments, the cancer-specific antigen (e.g., BCMA or CD38) binding Fab domain comprises different VH sequences. In some embodiments, the C-terminus of a cancer-specific antigen binding Fab domain is connected (e.g., covalently connected or chemically connected) to the N-terminus of a neighboring cancer-specific antigen binding Fab domain within the same multi-specific antibody.

The present disclosure also provides an antibody or antigen-binding fragment thereof that cross-competes with any antibody or antigen-binding fragment as described herein. The cross-competing assay is known in the art, and is described e.g., in Moore et al., “Antibody cross-competition analysis of the human immunodeficiency virus type 1 gp120 exterior envelope glycoprotein.” Journal of virology 70.3 (1996): 1863-1872, which is incorporated herein reference in its entirety. In one aspect, the present disclosure also provides an antibody or antigen-binding fragment thereof that binds to the same epitope or region as any antibody or antigen-binding fragment as described herein. The epitope binning assay is known in the art, and is described e.g., in Estep et al. “High throughput solution-based measurement of antibody-antigen affinity and epitope binning.” MAbs. Vol. 5. No. 2. Taylor & Francis, 2013, which is incorporated herein reference in its entirety.

### Antibodies and Antigen Binding Fragments

The present disclosure provides antibodies and antigen-binding fragments thereof that comprise complementary determining regions (CDRs), heavy chain variable regions, light chain variable regions, heavy chains, or light chains described herein. In some embodiments, the antibodies and antigen-binding fragments thereof are imbalanced bispecific antibodies and antigen-binding fragments thereof.

In general, antibodies (also called immunoglobulins) are made up of two classes of polypeptide chains, light chains and heavy chains. A non-limiting antibody of the present disclosure can be an intact, four immunoglobulin chain antibody comprising two heavy chains and two light chains. The heavy chain of the antibody can be of any isotype including IgM, IgG, IgE, IgA, or IgD or sub-isotype including IgG1, IgG2, IgG2a, IgG2b, IgG3, IgG4, IgE1, IgE2, etc. The light chain can be a kappa light chain or a lambda light chain. An antibody can comprise two identical copies of a light chain and/or two identical copies of a heavy chain. The heavy chains, which each contain one variable domain (or variable region, VH) and multiple constant domains (or constant regions), bind to one another via disulfide bonding within their constant domains to form the “stem” of the antibody. The light chains, which each contain one variable domain (or variable region, VL) and one constant domain (or constant region), each bind to one heavy chain via disulfide binding. The variable region of each light chain is aligned with the variable region of the heavy chain to which it is bound. The variable regions of both the light chains and heavy chains contain three hypervariable regions sandwiched between more conserved framework regions (FR).

These hypervariable regions, known as the complementary determining regions (CDRs), form loops that comprise the principle antigen binding surface of the antibody. The four framework regions largely adopt a beta-sheet conformation and the CDRs form loops connecting, and in some cases forming part of, the beta-sheet structure. The CDRs in each chain are held in close proximity by the framework regions and, with the CDRs from the other chain, contribute to the formation of the antigen-binding region.

Methods for identifying the CDR regions of an antibody by analyzing the amino acid sequence of the antibody are well known, and a number of definitions of the CDRs are commonly used. The Kabat definition is based on sequence variability, and the Chothia definition is based on the location of the structural loop regions. These methods and definitions are described in, e.g., Martin, “Protein sequence and structure analysis of antibody variable domains,” Antibody engineering, Springer Berlin Heidelberg, 2001. 422-439; Abhinandan, et al. “Analysis and improvements to Kabat and structurally correct numbering of antibody variable domains,” Molecular immunology 45.14 (2008): 3832-3839; Wu, T. T. and Kabat, E. A. (1970) J. Exp. Med. 132: 211-250; Martin et al., Methods Enzymol. 203:121-53 (1991); Morea et al., Biophys Chem. 68(1-3):9-16 (October 1997); Morea et al., J Mol Biol. 275(2):269-94 (January 0.1998); Chothia et al., Nature 342(6252):877-83 (December 1989); Ponomarenko and Bourne, BMC Structural Biology 7:64 (2007); Kontermann, R., & Dubel, S. (Eds.). (2010). Antibody engineering: Volume 2. Springer; each of which is incorporated herein by reference in its entirety. In some embodiments, the CDRs are based on Kabat definition. In some embodiments, the CDRs are based on the Chothia definition. In some embodiments, the CDRs are the longest CDR sequences as determined by Kabat, Chothia, AbM, IMGT, or contact definitions.

The CDRs are important for recognizing an epitope of an antigen. As used herein, an “epitope” is the smallest portion of a target molecule capable of being specifically bound by the antigen binding domain of an antibody. The minimal size of an epitope may be about three, four, five, six, or seven amino acids, but these amino acids need not be in a consecutive linear sequence of the antigen's primary structure, as the epitope may depend on an antigen's three-dimensional configuration based on the antigen's secondary and tertiary structure.

In some embodiments, the antibody is an intact immunoglobulin molecule (e.g., IgG1, IgG2a, IgG2b, IgG3, IgM, IgD, IgE, IgA). The IgG subclasses (IgG1, IgG2, IgG3, and IgG4) are highly conserved, differ in their constant region, particularly in their hinges and upper CH2 domains. The sequences and differences of the IgG subclasses are known in the art, and are described, e.g., in Vidarsson, et al, “IgG subclasses and allotypes: from structure to effector functions.” Frontiers in immunology 5 (2014); Irani, et al. “Molecular properties of human IgG subclasses and their implications for designing therapeutic monoclonal antibodies against infectious diseases.” Molecular immunology 67.2 (2015): 171-182; Shakib, Farouk, ed. The human IgG subclasses: molecular analysis of structure, function and regulation. Elsevier, 2016; each of which is incorporated herein by reference in its entirety.

The antibody can also be an immunoglobulin molecule that is derived from any species (e.g., human, rodent, mouse, rat, camelid). Antibodies disclosed herein also include, but are not limited to, polyclonal, monoclonal, monospecific, polyspecific antibodies, and chimeric antibodies that include an immunoglobulin binding domain fused to another polypeptide. The term “antigen binding domain” or “antigen binding fragment” is a portion of an antibody that retains specific binding activity of the intact antibody, i.e., any portion of an antibody that is capable of specific binding to an epitope on the intact antibody's target molecule. It includes, e.g., Fab, Fab′, F(ab′)2, and variants of these fragments. Thus, in some embodiments, an antibody or an antigen binding fragment thereof can be, e.g., a scFv, a Fv, a Fd, a dAb, a bispecific antibody, a bispecific scFv, a diabody, a linear antibody, a single-chain antibody molecule, a multi-specific antibody formed from antibody fragments, and any polypeptide that includes a binding domain which is, or is homologous to, an antibody binding domain. Non-limiting examples of antigen binding domains include, e.g., the heavy chain and/or light chain CDRs of an intact antibody, the heavy and/or light chain variable regions of an intact antibody, full length heavy or light chains of an intact antibody, or an individual CDR from either the heavy chain or the light chain of an intact antibody.

In some embodiments, the scFV has two heavy chain variable domains, and two light chain variable domains. In some embodiments, the scFV has two antigen binding regions (Antigen binding regions: A and B), and the two antigen binding regions can bind to the respective target antigens with different affinities.

In some embodiments, the antigen binding fragment can form a part of a chimeric antigen receptor (CAR). In some embodiments, the chimeric antigen receptor are fusions of single-chain variable fragments (scFv) as described herein, fused to CD3-zeta transmembrane- and endodomain. In some embodiments, the chimeric antigen receptor also comprises intracellular signaling domains from various costimulatory protein receptors (e.g., CD28, 41BB, ICOS). In some embodiments, the chimeric antigen receptor comprises multiple signaling domains, e.g., CD3z-CD28-41BB or CD3z-CD28-OX40, to increase potency. Thus, in one aspect, the disclosure further provides cells (e.g., T cells) that express the chimeric antigen receptors as described herein.

In some embodiments, the antibodies or antigen-binding fragments thereof can bind to two different antigens or two different epitopes. In some embodiments, the antibodies or antigen-binding fragments thereof can bind to three different antigens or three different epitopes.

An Fv fragment is an antibody fragment which contains a complete antigen recognition and binding site. This region consists of a dimer of one heavy and one light chain variable domain in tight association, which can be covalent in nature, for example in scFv. It is in this configuration that the three CDRs of each variable domain interact to define an antigen binding site on the surface of the VH-VL dimer. Collectively, the six CDRs or a subset thereof confer antigen binding specificity to the antibody. However, even a single variable domain (or half of an Fv comprising only three CDRs specific for an antigen) can have the ability to recognize and bind antigen, although usually at a lower affinity than the entire binding site.

Single-chain Fv or (scFv) antibody fragments comprise the VH and VL domains (or regions) of antibody, wherein these domains are present in a single polypeptide chain. Generally, the scFv polypeptide further comprises a polypeptide linker between the VH and VL domains, which enables the scFv to form the desired structure for antigen binding.

In some embodiments, the scFv described herein comprises from N-terminus to C-terminus: VH; the polypeptide linker; and VL. In some embodiments, the scFv described herein comprises from N-terminus to C-terminus: VL; the polypeptide linker; and VH. In some embodiments, the linker peptide comprises a sequence that is at least 80%, 85%, 90%, 95%, or 100% identical to any one SEQ ID NOs: 71-73 and 124-126. In some embodiments, the linker peptide comprises a sequence that is at least or about 80%, 85%, 90%, 95%, or 100% identical to SEQ ID NO: 72 or 73. In some embodiments, the linker peptide comprises a sequence that is at least or about 80%, 85%, 90%, 95%, or 100% identical to one or more (e.g., 1, 2, 3, 4, 5, 6, 7, or 8) repeats of GGGGS (SEQ ID NO: 71).

The Fab fragment contains a variable and constant domain of the light chain and a variable domain and the first constant domain (CHi) of the heavy chain. F(ab′)2 antibody fragments comprise a pair of Fab fragments which are generally covalently linked near their carboxy termini by hinge cysteines between them. Other chemical couplings of antibody fragments are also known in the art.

Diabodies are small antibody fragments with two antigen-binding sites, which fragments comprise a VH connected to a VL in the same polypeptide chain (VH and VL). By using a linker that is too short to allow pairing between the two domains on the same chain, the domains are forced to pair with the complementary domains of another chain and create two antigen-binding sites.

Multimerization of antibodies may be accomplished through natural aggregation of antibodies or through chemical or recombinant linking techniques known in the art. For example, some percentage of purified antibody preparations (e.g., purified IgG1 molecules) spontaneously form protein aggregates containing antibody homodimers and other higher-order antibody multimers.

Linear antibodies comprise a pair of tandem Fd segments (VH-CH1-VH-CH1) which, together with complementary light chain polypeptides, form a pair of antigen binding regions. Linear antibodies can be bispecific or monospecific.

Alternatively, antibody homodimers may be formed through chemical linkage techniques known in the art. For example, heterobifunctional crosslinking agents including, but not limited to SMCC (succinimidyl 4-(maleimidomethyl)cyclohexane-1-carboxylate) and SATA (N-succinimidyl S-acethylthio-acetate) can be used to form antibody multimers. An exemplary protocol for the formation of antibody homodimers is described in Ghetie et al. (Proc. Natl. Acad. Sci. U.S.A. 94: 7509-7514, 1997). Antibody homodimers can be converted to Fab′₂ homodimers through digestion with pepsin. Another way to form antibody homodimers is through the use of the autophilic T15 peptide described in Zhao et al. (J. Immunol. 25:396-404, 2002).

Antibodies and antibody fragments of the present disclosure can be modified in the Fc region to provide desired effector functions or serum half-life.

Any of the antibodies or antigen-binding fragments described herein may be conjugated to a stabilizing molecule (e.g., a molecule that increases the half-life of the antibody or antigen-binding fragment thereof in a subject or in solution). Non-limiting examples of stabilizing molecules include: a polymer (e.g., a polyethylene glycol) or a protein (e.g., serum albumin, such as human serum albumin). The conjugation of a stabilizing molecule can increase the half-life or extend the biological activity of an antibody or an antigen-binding fragment in vitro (e.g., in tissue culture or when stored as a pharmaceutical composition) or in vivo (e.g., in a human).

In some embodiments, the antibodies or antigen-binding fragments (e.g., bispecific antibodies) described herein can be conjugated to a therapeutic agent. The antibody-drug conjugate comprising the antibody or antigen-binding fragment thereof can covalently or non-covalently bind to a therapeutic agent. In some embodiments, the therapeutic agent is a cytotoxic or cytostatic agent (e.g., cytochalasin B, gramicidin D, ethidium bromide, emetine, mitomycin, etoposide, tenoposide, vincristine, vinblastine, colchicin, doxorubicin, daunorubicin, dihydroxy anthracin, maytansinoids such as DM-1 and DM-4, dione, mitoxantrone, mithramycin, actinomycin D, 1-dehydrotestosterone, glucocorticoids, procaine, tetracaine, lidocaine, propranolol, puromycin, epirubicin, and cyclophosphamide and analogs).

In some embodiments, the multispecific antibody or antigen-binding fragment thereof described herein increases T cell activation (e.g., as indicated by percentage of CD69+CD2+ cells, the percentage of CD69+ cells, or specific killing of target cells) by at least or about 10%, 20%, 30%, 40%, 50%, 60%, 70%, 80%, 90%, 100%, 2 folds, 3 folds, 4 folds, 5 folds, 6 folds, 7 folds, 8 folds, 9 folds, 10 folds, 20 folds, 30 folds, 40 folds, 50 folds, 100 folds, or more, as compared to an isotype control antibody, or a monoclonal antibody comprising the same VH or VL of the bispecific antibody. In some embodiments, the T cells are selected from Jurkat cells or PBMCs. In some embodiments, the target cells are selected from LS174T cells, SK-BR-3 cells, MDA231 cells, PANC1 cells, or Raji cells. In some embodiments, the target cells expresses cancer antigens (e.g., BCMA or CD38) endogenously or by transient transfection. In some embodiments, the T cells activation is mediated by the multi-specific antibody or antigen-binding fragment described herein at a concentration that is at least or about 0.1 μg/ml, 0.2 μg/ml, 0.3 μg/ml, 0.4 μg/ml, 0.5 μg/ml, 1 μg/ml, 2 μg/ml, 3 μg/ml, 4 μg/ml, 5 μg/ml, 6 μg/ml, 7 μg/ml, 8 μg/ml, 9 μg/ml, 10 μg/ml, 20 μg/ml, 30 μg/ml, 40 μg/ml, 50 μg/ml, or higher. In some embodiments, the T cells, target cells, and the multi-specific antibody are incubated for at least or about 8 hours, 16 hours, 24 hours, 36 hours, 48 hours, 60 hours, or 72 hours. In some embodiments, the ratio of the T cells and target cells is about 1:1, about 2:1, about 3:1, about 5:1, about 10:1, about 20:1, or about 25:1.

In some embodiments, the multi-specific antibody or antigen-binding fragment thereof described herein (e.g., a BCMA/CD3 bispecific antibody or a BCMA/CD38/CD3 trispecific antibody) binds to an antigen (e.g., BCMA) with a binding affinity that is about 50%, about 60%, about 70%, about 80%, about 90%, about 100%, about 110%, about 120%, about 130%, about 140%, about 150%, or about 200% to that of a heavy-chain antibody (e.g., an anti-BCMA heavy-chain antibody) comprising the same VHH of the multi-specific antibody.

In some embodiments, the multi-specific antibody or antigen-binding fragment thereof described herein (e.g., a CD38/CD3 bispecific antibody or a BCMA/CD38/CD3 trispecific antibody) binds to an antigen (e.g., CD38) with a binding affinity that is about 50%, about 60%, about 70%, about 80%, about 90%, about 100%, about 110%, about 120%, about 130%, about 140%, about 150%, or about 200% to that of a heavy-chain antibody (e.g., an anti-CD38 heavy-chain antibody) comprising the same VHH of the multi-specific antibody.

In some embodiments, the bispecific antibody or antigen-binding fragment thereof described herein (e.g., a BCMA/CD3 bispecific antibody, a CD38/CD3 bispecific antibody, or a BCMA/CD38/CD3 trispecific antibody) mediates complement-dependent cytotoxicity (CDC) to at least or about 1 fold, 2 folds, 3 folds, 4 folds, 5 folds, 6 folds, 7 folds, 8 folds, 9 folds, 10 folds, 11 folds, 12 folds, 13 folds, 14 folds, 15 folds, 16 folds, 17 folds, 18 folds, 19 folds, 20 folds, 30 folds, 40 folds, or 50 folds as compared to that mediated by an isotype control antibody.

In some embodiments, the multi-specific antibody or antigen-binding fragment thereof described herein (e.g., a BCMA/CD3 bispecific antibody, a CD38/CD3 bispecific antibody, or a BCMA/CD38/CD3 trispecific antibody) is internalized at a percentage that is about 50%, about 60%, about 70%, about 80%, about 90%, about 100%, about 110%, about 120%, about 130%, about 140%, about 150%, or about 200% to that of a monoclonal antibody (e.g., an anti-BCMA antibody, an anti-CD38 antibody, an anti-CD3 antibody, or an isotype antibody control).

### Recombinant Vectors

The present disclosure also provides recombinant vectors (e.g., an expression vectors) that include an isolated polynucleotide disclosed herein (e.g., a polynucleotide that encodes a polypeptide disclosed herein), host cells into which are introduced the recombinant vectors (i.e., such that the host cells contain the polynucleotide and/or a vector comprising the polynucleotide), and the production of recombinant antibody polypeptides or fragments thereof by recombinant techniques.

As used herein, a “vector” is any construct capable of delivering one or more polynucleotide(s) of interest to a host cell when the vector is introduced to the host cell. An “expression vector” is capable of delivering and expressing the one or more polynucleotide(s) of interest as an encoded polypeptide in a host cell into which the expression vector has been introduced. Thus, in an expression vector, the polynucleotide of interest is positioned for expression in the vector by being operably linked with regulatory elements such as a promoter, enhancer, and/or a poly-A tail, either within the vector or in the genome of the host cell at or near or flanking the integration site of the polynucleotide of interest such that the polynucleotide of interest will be translated in the host cell introduced with the expression vector.

A vector can be introduced into the host cell by methods known in the art, e.g., electroporation, chemical transfection (e.g., DEAE-dextran), transformation, transfection, and infection and/or transduction (e.g., with recombinant virus). Thus, non-limiting examples of vectors include viral vectors (which can be used to generate recombinant virus), naked DNA or RNA, plasmids, cosmids, phage vectors, and DNA or RNA expression vectors associated with cationic condensing agents.

In some implementations, a polynucleotide disclosed herein (e.g., a polynucleotide that encodes a polypeptide disclosed herein) is introduced using a viral expression system (e.g., vaccinia or other pox virus, retrovirus, or adenovirus), which may involve the use of a non-pathogenic (defective), replication competent virus, or may use a replication defective virus. In the latter case, viral propagation generally will occur only in complementing virus packaging cells. Suitable systems are disclosed, for example, in Fisher-Hoch et al., 1989, Proc. Natl. Acad. Sci. USA 86:317-321; Flexner et al., 1989, Ann. N.Y. Acad Sci. 569:86-103; Flexner et al., 1990, Vaccine, 8:17-21; U.S. Pat. Nos. 4,603,112, 4,769,330, and 5,017,487; WO 89/01973; U.S. Pat. No. 4,777,127; GB 2,200,651; EP 0,345,242; WO 91/02805; Berkner-Biotechniques, 6:616-627, 1988; Rosenfeld et al., 1991, Science, 252:431-434; Kolls et al., 1994, Proc. Natl. Acad. Sci. USA, 91:215-219; Kass-Eisler et al., 1993, Proc. Natl. Acad. Sci. USA, 90:11498-11502; Guzman et al., 1993, Circulation, 88:2838-2848; and Guzman et al., 1993, Cir. Res., 73:1202-1207. Techniques for incorporating DNA into such expression systems are well known to those of ordinary skill in the art. The DNA may also be “naked,” as described, for example, in Ulmer et al., 1993, Science, 259:1745-1749, and Cohen, 1993, Science, 259:1691-1692. The uptake of naked DNA may be increased by coating the DNA onto biodegradable beads that are efficiently transported into the cells.

For expression, the DNA insert comprising an antibody-encoding or polypeptide-encoding polynucleotide disclosed herein can be operatively linked to an appropriate promoter (e.g., a heterologous promoter), such as the phage lambda PL promoter, the E. coli lac, trp and tac promoters, the SV40 early and late promoters and promoters of retroviral LTRs, to name a few. Other suitable promoters are known to the skilled artisan. The expression constructs can further contain sites for transcription initiation, termination and, in the transcribed region, a ribosome binding site for translation. The coding portion of the mature transcripts expressed by the constructs may include a translation initiating at the beginning and a termination codon (UAA, UGA, or UAG) appropriately positioned at the end of the polypeptide to be translated.

As indicated, the expression vectors can include at least one selectable marker. Such markers include dihydrofolate reductase or neomycin resistance for eukaryotic cell culture and tetracycline or ampicillin resistance genes for culturing in E. coli and other bacteria. Representative examples of appropriate hosts include, but are not limited to, bacterial cells, such as E. coli, Streptomyces, and Salmonella typhimurium cells; fungal cells, such as yeast cells; insect cells such as Drosophila S2 and Spodoptera Sf9 cells; animal cells such as CHO, COS, Bowes melanoma, and HK 293 cells; and plant cells. Appropriate culture mediums and conditions for the host cells described herein are known in the art.

Non-limiting vectors for use in bacteria include pQE70, pQE60 and pQE-9, available from Qiagen; pBS vectors, Phagescript vectors, Bluescript vectors, pNH8A, pNH16a, pNH18A, pNH46A, available from Stratagene; and ptrc99a, pKK223-3, pKK233-3, pDR540, pRIT5 available from Pharmacia. Non-limiting eukaryotic vectors include pWLNEO, pSV2CAT, pOG44, pXT1 and pSG available from Stratagene; and pSVK3, pBPV, pMSG and pSVL available from Pharmacia. Other suitable vectors will be readily apparent to the skilled artisan.

Non-limiting bacterial promoters suitable for use include the E. coli lacI and lacZ promoters, the T3 and T7 promoters, the gpt promoter, the lambda PR and PL promoters and the trp promoter. Suitable eukaryotic promoters include the CMV immediate early promoter, the HSV thymidine kinase promoter, the early and late SV40 promoters, the promoters of retroviral LTRs, such as those of the Rous sarcoma virus (RSV), and metallothionein promoters, such as the mouse metallothionein-I promoter.

In the yeast Saccharomyces cerevisiae, a number of vectors containing constitutive or inducible promoters such as alpha factor, alcohol oxidase, and PGH may be used. For reviews, see Ausubel et al. (1989) Current Protocols in Molecular Biology, John Wiley & Sons, New York, N.Y., and Grant et al., Methods Enzymol., 153: 516-544 (1997).

Introduction of the construct into the host cell can be effected by calcium phosphate transfection, DEAE-dextran mediated transfection, cationic lipid-mediated transfection, electroporation, transduction, infection or other methods. Such methods are described in many standard laboratory manuals, such as Davis et al., Basic Methods In Molecular Biology (1986), which is incorporated herein by reference in its entirety.

Transcription of DNA encoding an antibody of the present disclosure by higher eukaryotes may be increased by inserting an enhancer sequence into the vector. Enhancers are cis-acting elements of DNA, usually about from 10 to 300 bp that act to increase transcriptional activity of a promoter in a given host cell-type. Examples of enhancers include the SV40 enhancer, which is located on the late side of the replication origin at base pairs 100 to 270, the cytomegalovirus early promoter enhancer, the polyoma enhancer on the late side of the replication origin, and adenovirus enhancers.

For secretion of the translated protein into the lumen of the endoplasmic reticulum, into the periplasmic space or into the extracellular environment, appropriate secretion signals may be incorporated into the expressed polypeptide. The signals may be endogenous to the polypeptide or they may be heterologous signals.

The polypeptide (e.g., antibody) can be expressed in a modified form, such as a fusion protein (e.g., a GST-fusion) or with a histidine-tag, and may include not only secretion signals, but also additional heterologous functional regions. For instance, a region of additional amino acids, particularly charged amino acids, may be added to the N-terminus of the polypeptide to improve stability and persistence in the host cell, during purification, or during subsequent handling and storage. Also, peptide moieties can be added to the polypeptide to facilitate purification. Such regions can be removed prior to final preparation of the polypeptide. The addition of peptide moieties to polypeptides to engender secretion or excretion, to improve stability and to facilitate purification, among others, are familiar and routine techniques in the art.

The disclosure also provides a nucleic acid sequence that is at least 1%, 2%, 3%, 4%, 5%, 6%, 7%, 8%, 9%, 10%, 15%, 20%, 25%, 30%, 35%0, 40%, 45%, 50%, 55%, 60%, 65%, 70%, 75%, 80%, 85%, 90%, 91%, 92%, 93%, 94%, 95%, 96%, 97%, 98%, 99% identical to any nucleotide sequence as described herein, and an amino acid sequence that is at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% identical to any amino acid sequence as described herein.

The disclosure also provides a nucleic acid sequence that has a homology of at least 1%, 2%, 3%, 4%, 5%, 6%7, 7%, 8%, 9%, 10, 1%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% to any nucleotide sequence as described herein, and an amino acid sequence that has a homology of at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% to any amino acid sequence as described herein.

In some embodiments, the disclosure relates to nucleotide sequences encoding any peptides that are described herein, or any amino acid sequences that are encoded by any nucleotide sequences as described herein. In some embodiments, the nucleic acid sequence is less than 10, 20, 30, 40, 50, 60, 70, 80, 90, 100, 110, 120, 130, 150, 200, 250, 300, 350, 400, 500, or 600 nucleotides. In some embodiments, the amino acid sequence is less than 5, 6, 7, 8, 9, 10, 20, 30, 40, 50, 60, 70, 80, 90, 100, 110, 120, 130, 140, 150, 160, 170, 180, 190, 200, 250, 300, 350, or 400 amino acid residues.

In some embodiments, the amino acid sequence (i) comprises an amino acid sequence; or (ii) consists of an amino acid sequence, wherein the amino acid sequence is any one of the sequences as described herein.

In some embodiments, the nucleic acid sequence (i) comprises a nucleic acid sequence; or (ii) consists of a nucleic acid sequence, wherein the nucleic acid sequence is any one of the sequences as described herein.

The percentage of sequence homology (e.g., amino acid sequence homology or nucleic acid homology) can also be determined. How to determine percentage of sequence homology is known in the art. In some embodiments, amino acid residues conserved with similar physicochemical properties (percent homology), e.g. leucine and isoleucine, can be used to measure sequence similarity. Families of amino acid residues having similar physicochemical properties have been defined in the art. These families include e.g., amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). The homology percentage, in many cases, is higher than the identity percentage.

### Methods of Making Antibodies

An isolated fragment of human protein (e.g., BCMA, CD38, CD3, or cancer antigens) can be used as an immunogen to generate antibodies using standard techniques for polyclonal and monoclonal antibody preparation. Polyclonal antibodies can be raised in animals by multiple injections (e.g., subcutaneous or intraperitoneal injections) of an antigenic peptide or protein. In some embodiments, the antigenic peptide or protein is injected with at least one adjuvant. In some embodiments, the antigenic peptide or protein can be conjugated to an agent that is immunogenic in the species to be immunized. Animals can be injected with the antigenic peptide or protein more than one time (e.g., twice, three times, or four times).

The full-length polypeptide or protein can be used or, alternatively, antigenic peptide fragments thereof can be used as immunogens. The antigenic peptide of a protein comprises at least 8 (e.g., at least 10, 15, 20, or 30) amino acid residues of the amino acid sequence of the protein and encompasses an epitope of the protein such that an antibody raised against the peptide forms a specific immune complex with the protein.

An immunogen typically is used to prepare antibodies by immunizing a suitable subject (e.g., human or transgenic animal expressing at least one human immunoglobulin locus). An appropriate immunogenic preparation can contain, for example, a recombinantly-expressed or a chemically-synthesized polypeptide. The preparation can further include an adjuvant, such as Freund's complete or incomplete adjuvant, or a similar immunostimulatory agent.

Polyclonal antibodies can be prepared as described above by immunizing a suitable subject with a polypeptide, or an antigenic peptide thereof (e.g., part of the protein) as an immunogen. The antibody titer in the immunized subject can be monitored over time by standard techniques, such as with an enzyme-linked immunosorbent assay (ELISA) using the immobilized polypeptide or peptide. If desired, the antibody molecules can be isolated from the mammal (e.g., from the blood) and further purified by well-known techniques, such as protein A of protein G chromatography to obtain the IgG fraction. At an appropriate time after immunization, e.g., when the specific antibody titers are highest, antibody-producing cells can be obtained from the subject and used to prepare monoclonal antibodies by standard techniques, such as the hybridoma technique originally described by Kohler et al. (Nature 256:495-497, 1975), the human B cell hybridoma technique (Kozbor et al., Immunol. Today 4:72, 1983), the EBV-hybridoma technique (Cole et al., Monoclonal Antibodies and Cancer Therapy, Alan R. Liss, Inc., pp. 77-96, 1985), or trioma techniques. The technology for producing hybridomas is well known (see, generally, Current Protocols in Immunology, 1994, Coligan et al. (Eds.), John Wiley & Sons, Inc., New York, NY). Hybridoma cells producing a monoclonal antibody are detected by screening the hybridoma culture supernatants for antibodies that bind the polypeptide or epitope of interest, e.g., using a standard ELISA assay.

VHH can also be obtained from naïve or designed synthetic llama VHH libraries. PBMC from llamas can be obtained, and RNA can be isolated to generate cDNA by reverse transcription. Then, the VHH genes can be amplified by PCR and cloned to a phage display vector to construct the naïve VHH library. The synthetic (e.g., humanized) VHH library can be prepared by incorporation of shuffled VHH CDR1, 2 and 3, generated by overlapping PCR, to a modified human VH scaffold to generate enhanced diversity and keep low immunogenicity. The VHH libraries can be then panned against antigens to obtain VHH with desired binding affinities.

Variants of the antibodies or antigen-binding fragments described herein can be prepared by introducing appropriate nucleotide changes into the DNA encoding a human, humanized, or chimeric antibody, or antigen-binding fragment thereof described herein, or by peptide synthesis. Such variants include, for example, deletions, insertions, or substitutions of residues within the amino acids sequences that make-up the antigen-binding site of the antibody or an antigen-binding domain. In a population of such variants, some antibodies or antigen-binding fragments will have increased affinity for the target protein. Any combination of deletions, insertions, and/or combinations can be made to arrive at an antibody or antigen-binding fragment thereof that has increased binding affinity for the target. The amino acid changes introduced into the antibody or antigen-binding fragment can also alter or introduce new post-translational modifications into the antibody or antigen-binding fragment, such as changing (e.g., increasing or decreasing) the number of glycosylation sites, changing the type of glycosylation site (e.g., changing the amino acid sequence such that a different sugar is attached by enzymes present in a cell), or introducing new glycosylation sites.

Antibodies disclosed herein can be derived from any species of animal, including mammals. Non-limiting examples of native antibodies include antibodies derived from humans, primates, e.g., monkeys and apes, cows, pigs, horses, sheep, camelids (e.g., camels and llamas), chicken, goats, and rodents (e.g., rats, mice, hamsters and rabbits), including transgenic rodents genetically engineered to produce human antibodies.

Phage display (panning) can be used to optimize antibody sequences with desired binding affinities. In this technique, a gene encoding single chain Fv (comprising VH or VL) can be inserted into a phage coat protein gene, causing the phage to “display” the scFv on its outside while containing the gene for the protein on its inside, resulting in a connection between genotype and phenotype. These displaying phages can then be screened against target antigens, in order to detect interaction between the displayed antigen binding sites and the target antigen. Thus, large libraries of proteins can be screened and amplified in a process called in vitro selection, and antibodies sequences with desired binding affinities can be obtained.

Human and humanized antibodies include antibodies having variable and constant regions derived from (or having the same amino acid sequence as those derived from) human germline immunoglobulin sequences. Human antibodies may include amino acid residues not encoded by human germline immunoglobulin sequences (e.g., mutations introduced by random or site-specific mutagenesis in vitro or by somatic mutation in vivo), for example in the CDRs.

A humanized antibody, typically has a human framework (FR) grafted with non-human CDRs. Thus, a humanized antibody has one or more amino acid sequence introduced into it from a source which is non-human. These non-human amino acid residues are often referred to as “import” residues, which are typically taken from an “import” variable domain. Humanization can be essentially performed by e.g., substituting rodent CDRs or CDR sequences for the corresponding sequences of a human antibody. These methods are described in e.g., Jones et al., Nature, 321:522-525 (1986); Riechmann et al., Nature, 332:323-327 (1988); Verhoeyen et al., Science, 239:1534-1536 (1988); each of which is incorporated by reference herein in its entirety. Accordingly, “humanized” antibodies are chimeric antibodies wherein substantially less than an intact human V domain has been substituted by the corresponding sequence from a non-human species. In practice, humanized antibodies are typically mouse antibodies in which some CDR residues and some FR residues are substituted by residues from analogous sites in human antibodies.

It is further important that antibodies be humanized with retention of high specificity and affinity for the antigen and other favorable biological properties. To achieve this goal, humanized antibodies can be prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved.

Identity or homology with respect to an original sequence is usually the percentage of amino acid residues present within the candidate sequence that are identical with a sequence present within the human, humanized, or chimeric antibody or fragment, after aligning the sequences and introducing gaps, if necessary, to achieve the maximum percent sequence identity, and not considering any conservative substitutions as part of the sequence identity.

In some embodiments, a covalent modification can be made to the antibody or antigen-binding fragment thereof. These covalent modifications can be made by chemical or enzymatic synthesis, or by enzymatic or chemical cleavage. Other types of covalent modifications of the antibody or antibody fragment are introduced into the molecule by reacting targeted amino acid residues of the antibody or fragment with an organic derivatization agent that is capable of reacting with selected side chains or the N- or C-terminal residues.

In some embodiments, antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g. complex, hybrid and high mannose structures) as measured by MALDI-TOF mass spectrometry, as described in WO 2008/077546, for example. Asn297 refers to the asparagine residue located at about position 297 in the Fc region (Eu numbering of Fc region residues; or position 314 in Kabat numbering); however, Asn297 may also be located about ±3 amino acids upstream or downstream of position 297, i.e., between positions 294 and 300, due to minor sequence variations in antibodies. Such fucosylation variants may have improved ADCC function. In some embodiments, to reduce glycan heterogeneity, the Fc region of the antibody can be further engineered to replace the Asparagine at position 297 with Alanine (N297A).

In some embodiments, to facilitate production efficiency by avoiding Fab-arm exchange, the Fc region of the antibodies was further engineered to replace the serine at position 228 (EU numbering) of IgG4 with proline (S228P). A detailed description regarding S228 mutation is described, e.g., in Silva et al. “The S228P mutation prevents in vivo and in vitro IgG4 Fab-arm exchange as demonstrated using a combination of novel quantitative immunoassays and physiological matrix preparation.” Journal of Biological Chemistry 290.9 (2015): 5462-5469, which is incorporated by reference in its entirety.

In some embodiments, the methods described here are designed to make a bispecific antibody. Bispecific antibodies can be made by engineering the interface between a pair of antibody molecules to maximize the percentage of heterodimers that are recovered from recombinant cell culture. For example, the interface can contain at least a part of the CH3 domain of an antibody constant domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers. This method is described, e.g., in WO 96/27011, which is incorporated by reference in its entirety.

In some embodiments, one or more amino acid residues in the CH3 portion of the IgG are substituted. In some embodiments, one heavy chain has one or more of the following substitutions Y349C and T366W. The other heavy chain can have one or more the following substitutions E356C, T366S, L368A, and Y407V. Furthermore, a substitution (-ppcpScp-->-ppcpPcp-) can also be introduced at the hinge regions of both substituted IgG. In some embodiments, one heavy chain has a T366Y (knob) substitution, and the other heavy chain has a Y407T (hole) substitution (EU numbering).

One aspect of the present application provides a heteromultimeric (e.g., heterodimeric) protein comprising a first polypeptide comprising a first heavy chain constant domain 3 (CH3) domain and a second polypeptide comprising a second CH3 domain, wherein the first CH3 domain comprises a substitution relative to a wildtype CH3 domain at amino acid position 354 with a bulky hydrophobic amino acid, and/or the second CH3 domain comprises a substitution relative to a wildtype CH3 domain at amino acid position 347 with a negatively charged amino acid, and wherein the amino acid residue numbering is based on EU numbering. In some embodiments, the bulky hydrophobic amino acid at amino acid position 354 forms a hydrophobic interaction with an amino acid residue in the second CH3 domain. In some embodiments, the second CH3 domain comprises a bulky hydrophobic residue at amino acid position 349 (e.g., Y349). In some embodiments, the negatively charged amino acid at amino acid position 347 forms an ionic bond with an amino acid residue in the first CH3 domain. In some embodiments, the first CH3 domain comprises a positively charged residue at amino acid position 360 (e.g., K360). In some embodiments, the first CH3 domain and the second CH3 domain are human CH3 domains. In some embodiments, the first CH3 domain comprises a substitution selected from the group consisting of S354Y, S354F and S354W. In some embodiments, the first CH3 domain comprises S354Y. In some embodiments, the second CH3 domain does not comprise a compensatory substitution (e.g., a substitution at Y349) for the substitution of S354 in the first CH3 domain. In some embodiments, the second CH3 domain comprises a substitution selected from the group consisting of Q347E and Q347D. In some embodiments, the second CH3 domain comprises Q347E. In some embodiments according to any one of the heteromultimeric proteins described above, the first CH3 domain and the second CH3 domain further comprise knob-into-hole (KIH) residues. In some embodiments, the knob-into-hole residues are T366Y and Y407T. In some embodiments, the first CH3 domain comprises T366Y and S354Y, and the second CH3 domain comprises Y407T and Q347E. In some embodiments, the first CH3 domain comprises Y407T and S354Y, and the second CH3 domain comprises T366Y and Q347E. Details can be found, e.g., in PCT/US2020/025469, which is incorporated herein by reference.

Furthermore, an anion-exchange chromatography can be used to purify bispecific antibodies. Anion-exchange chromatography is a process that separates substances based on their charges using an ion-exchange resin containing positively charged groups, such as diethyl-aminoethyl groups (DEAE). In solution, the resin is coated with positively charged counter-ions (cations). Anion exchange resins will bind to negatively charged molecules, displacing the counter-ion. Anion exchange chromatography can be used to purify proteins based on their isoelectric point (pI). The isoelectric point is defined as the pH at which a protein has no net charge. When the pH>pI, a protein has a net negative charge and when the pH<pI, a protein has a net positive charge. Thus, in some embodiments, different amino acid substitution can be introduced into two heavy chains, so that the pI for the homodimer comprising two Arm A and the pI for the homodimer comprising two Arm B is different. The pI for the bispecific antibody having Arm A and Arm B will be somewhere between the two pIs of the homodimers. Thus, the two homodimers and the bispecific antibody can be released at different pH conditions. The present disclosure shows that a few amino acid residue substitutions can be introduced to the heavy chains to adjust pI.

Thus, in some embodiments, the amino acid residue at Kabat numbering position 83 is lysine, arginine, or histidine. In some embodiments, the amino acid residues at one or more of the positions 1, 6, 43, 81, and 105 (Kabat numbering) is aspartic acid or glutamic acid.

In some embodiments, the amino acid residues at one or more of the positions 13 and 105 (Kabat numbering) is aspartic acid or glutamic acid. In some embodiments, the amino acid residues at one or more of the positions 13 and 42 (Kabat numbering) is lysine, arginine, histidine, or glycine.

Bispecific antibodies can also include e.g., cross-linked or “heteroconjugate” antibodies. For example, one of the antibodies in the heteroconjugate can be coupled to avidin and the other to biotin. Heteroconjugate antibodies can also be made using any convenient cross-linking methods. Suitable cross-linking agents and cross-linking techniques are well known in the art and are disclosed in U.S. Pat. No. 4,676,980, which is incorporated herein by reference in its entirety.

Methods for generating bispecific antibodies from antibody fragments are also known in the art. For example, bispecific antibodies can be prepared using chemical linkage. Brennan et al. (Science 229:81, 1985) describes a procedure where intact antibodies are proteolytically cleaved to generate F(ab′)2 fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′ TNB derivatives is then reconverted to the Fab′ thiol by reduction with mercaptoethylamine, and is mixed with an equimolar amount of another Fab′ TNB derivative to form the bispecific antibody.

### Methods of Treatment

The methods described herein include methods for the treatment of disorders associated with cancer. Generally, the methods include administering a therapeutically effective amount of engineered bispecific antibodies (e.g., imbalanced bispecific antibodies) of antigen-binding fragments thereof as described herein, to a subject who is in need of, or who has been determined to be in need of, such treatment.

As used in this context, to “treat” means to ameliorate at least one symptom of the disorder associated with cancer. Often, cancer results in death; thus, a treatment can result in an increased life expectancy (e.g., by at least 1, 2, 3, 4, 5, 6, 7, 8, 9, 10, 11, 12 months, or by at least 1, 2, 3, 4, 5, 6, 7, 8, 9, 10 years). Administration of a therapeutically effective amount of an agent described herein (e.g., imbalanced bispecific antibodies) for the treatment of a condition associated with cancer will result in decreased number of cancer cells and/or alleviated symptoms.

As used herein, the term “cancer” refers to cells having the capacity for autonomous growth, i.e., an abnormal state or condition characterized by rapidly proliferating cell growth. The term is meant to include all types of cancerous growths or oncogenic processes, metastatic tissues or malignantly transformed cells, tissues, or organs, irrespective of histopathologic type or stage of invasiveness. The term “tumor” as used herein refers to cancerous cells, e.g., a mass of cancerous cells. Cancers that can be treated or diagnosed using the methods described herein include malignancies of the various organ systems, such as affecting lung, breast, thyroid, lymphoid, gastrointestinal, and genito-urinary tract, as well as adenocarcinomas which include malignancies such as most colon cancers, renal-cell carcinoma, prostate cancer and/or testicular tumors, non-small cell carcinoma of the lung, cancer of the small intestine and cancer of the esophagus. In some embodiments, the agents described herein are designed for treating or diagnosing a carcinoma in a subject. The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. In some embodiments, the cancer is renal carcinoma or melanoma. Exemplary carcinomas include those forming from tissue of the cervix, lung, prostate, breast, head and neck, colon and ovary. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures. The term “sarcoma” is art recognized and refers to malignant tumors of mesenchymal derivation.

In one aspect, the disclosure also provides methods for treating a cancer in a subject, methods of reducing the rate of the increase of volume of a tumor in a subject over time, methods of reducing the risk of developing a metastasis, or methods of reducing the risk of developing an additional metastasis in a subject. In some embodiments, the treatment can halt, slow, retard, or inhibit progression of a cancer. In some embodiments, the treatment can result in the reduction of in the number, severity, and/or duration of one or more symptoms of the cancer in a subject.

In one aspect, the disclosure features methods that include administering a therapeutically effective amount of an antibody or antigen-binding fragment thereof, or an antibody drug conjugate disclosed herein to a subject in need thereof, e.g., a subject having, or identified or diagnosed as having, a cancer, e.g., breast cancer (e.g., triple-negative breast cancer), carcinoid cancer, cervical cancer, endometrial cancer, glioma, head and neck cancer, liver cancer, lung cancer, small cell lung cancer, lymphoma, melanoma, ovarian cancer, pancreatic cancer, prostate cancer, renal cancer, colorectal cancer, gastric cancer, testicular cancer, thyroid cancer, bladder cancer, urethral cancer, or hematologic malignancy.

As used herein, the terms “subject” and “patient” are used interchangeably throughout the specification and describe an animal, human or non-human, to whom treatment according to the methods of the present invention is provided. Veterinary and non-veterinary applications are contemplated by the present invention. Human patients can be adult humans or juvenile humans (e.g., humans below the age of 18 years old). In addition to humans, patients include but are not limited to mice, rats, hamsters, guinea-pigs, rabbits, ferrets, cats, dogs, and primates. Included are, for example, non-human primates (e.g., monkey, chimpanzee, gorilla, and the like), rodents (e.g., rats, mice, gerbils, hamsters, ferrets, rabbits), lagomorphs, swine (e.g., pig, miniature pig), equine, canine, feline, bovine, and other domestic, farm, and zoo animals.

In some embodiments, the cancer is a cancer expressing BCMA. In some embodiments, the cancer is a cancer expressing CD38. In some embodiments, the cancer is a cancer expressing BCMA. In some embodiments, the cancer is a cancer expressing both BCMA and CD38.

In some embodiments, the cancers are hematological malignancies (e.g., multiple myeloma).

In some embodiments, the cancer is unresectable melanoma or metastatic melanoma, non-small cell lung carcinoma (NSCLC), small cell lung cancer (SCLC), bladder cancer, or metastatic hormone-refractory prostate cancer. In some embodiments, the subject has a solid tumor. In some embodiments, the cancer is squamous cell carcinoma of the head and neck (SCCHN), renal cell carcinoma (RCC), triple-negative breast cancer (TNBC), or colorectal carcinoma. In some embodiments, the subject has Hodgkin's lymphoma. In some embodiments, the subject has triple-negative breast cancer (TNBC), gastric cancer, urothelial cancer, Merkel-cell carcinoma, or head and neck cancer. In some embodiments, the cancer is melanoma, pancreatic carcinoma, mesothelioma, hematological malignancies, especially Non-Hodgkin's lymphoma, lymphoma, chronic lymphocytic leukemia, or advanced solid tumors.

In some embodiments, the compositions and methods disclosed herein can be used for treatment of patients at risk for a cancer. Patients with cancer can be identified with various methods known in the art.

As used herein, by an “effective amount” is meant an amount or dosage sufficient to effect beneficial or desired results including halting, slowing, retarding, or inhibiting progression of a disease, e.g., a cancer. An effective amount will vary depending upon, e.g., an age and a body weight of a subject to which the antibody, antigen binding fragment, antibody-drug conjugates, antibody-encoding polynucleotide, vector comprising the polynucleotide, and/or compositions thereof is to be administered, a severity of symptoms and a route of administration, and thus administration can be determined on an individual basis.

An effective amount can be administered in one or more administrations. By way of example, an effective amount of an antibody, an antigen binding fragment, or an antibody-drug conjugate is an amount sufficient to ameliorate, stop, stabilize, reverse, inhibit, slow and/or delay progression of an autoimmune disease or a cancer in a patient or is an amount sufficient to ameliorate, stop, stabilize, reverse, slow and/or delay proliferation of a cell (e.g., a biopsied cell, any of the cancer cells described herein, or cell line (e.g., a cancer cell line)) in vitro. As is understood in the art, an effective amount of an antibody, antigen binding fragment, or antibody-drug conjugate may vary, depending on, inter alia, patient history as well as other factors such as the type (and/or dosage) of antibody used.

Effective amounts and schedules for administering the antibodies, antibody-encoding polynucleotides, antibody-drug conjugates, and/or compositions disclosed herein may be determined empirically, and making such determinations is within the skill in the art. Those skilled in the art will understand that the dosage that must be administered will vary depending on, for example, the mammal that will receive the antibodies, antibody-encoding polynucleotides, antibody-drug conjugates, and/or compositions disclosed herein, the route of administration, the particular type of antibodies, antibody-encoding polynucleotides, antigen binding fragments, antibody-drug conjugates, and/or compositions disclosed herein used and other drugs being administered to the mammal. Guidance in selecting appropriate doses for antibody or antigen binding fragment can be found in the literature on therapeutic uses of antibodies and antigen binding fragments, e.g., Handbook of Monoclonal Antibodies, Ferrone et al., eds., Noges Publications, Park Ridge, N.J., 1985, ch. 22 and pp. 303-357; Smith et al., Antibodies in Human Diagnosis and Therapy, Haber et al., eds., Raven Press, New York, 1977, pp. 365-389.

A typical daily dosage of an effective amount of an antibody is 0.01 mg/kg to 100 mg/kg. In some embodiments, the dosage can be less than 100 mg/kg, 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.5 mg/kg, or 0.1 mg/kg. In some embodiments, the dosage can be greater than 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.5 mg/kg, 0.1 mg/kg, 0.05 mg/kg, or 0.01 mg/kg. In some embodiments, the dosage is about 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.9 mg/kg, 0.8 mg/kg, 0.7 mg/kg, 0.6 mg/kg, 0.5 mg/kg, 0.4 mg/kg, 0.3 mg/kg, 0.2 mg/kg, or 0.1 mg/kg.

In any of the methods described herein, the at least one antibody, antigen-binding fragment thereof, antibody-drug conjugates, or pharmaceutical composition (e.g., any of the antibodies, antigen-binding fragments, antibody-drug conjugates, or pharmaceutical compositions described herein) and, optionally, at least one additional therapeutic agent can be administered to the subject at least once a week (e.g., once a week, twice a week, three times a week, four times a week, once a day, twice a day, or three times a day). In some embodiments, at least two different antibodies and/or antigen-binding fragments are administered in the same composition (e.g., a liquid composition). In some embodiments, at least one antibody, antigen-binding fragment, antibody-drug conjugates, and at least one additional therapeutic agent are administered in the same composition (e.g., a liquid composition). In some embodiments, the at least one antibody or antigen-binding fragment and the at least one additional therapeutic agent are administered in two different compositions (e.g., a liquid composition containing at least one antibody or antigen-binding fragment and a solid oral composition containing at least one additional therapeutic agent). In some embodiments, the at least one additional therapeutic agent is administered as a pill, tablet, or capsule. In some embodiments, the at least one additional therapeutic agent is administered in a sustained-release oral formulation.

In some embodiments, the one or more additional therapeutic agents can be administered to the subject prior to, or after administering the at least one antibody, antigen-binding antibody fragment, antibody-drug conjugate, or pharmaceutical composition (e.g., any of the antibodies, antigen-binding antibody fragments, or pharmaceutical compositions described herein). In some embodiments, the one or more additional therapeutic agents and the at least one antibody, antigen-binding antibody fragment, antibody-drug conjugate, or pharmaceutical composition (e.g., any of the antibodies, antigen-binding antibody fragments, or pharmaceutical compositions described herein) are administered to the subject such that there is an overlap in the bioactive period of the one or more additional therapeutic agents and the at least one antibody or antigen-binding fragment (e.g., any of the antibodies or antigen-binding fragments described herein) in the subject.

In some embodiments, the subject can be administered the at least one antibody, antigen-binding antibody fragment, antibody-drug conjugate, or pharmaceutical composition (e.g., any of the antibodies, antigen-binding antibody fragments, or pharmaceutical compositions described herein) over an extended period of time (e.g., over a period of at least 1 week, 2 weeks, 3 weeks, 1 month, 2 months, 3 months, 4 months, 5 months, 6 months, 7 months, 8 months, 9 months, 10 months, 11 months, 12 months, 1 year, 2 years, 3 years, 4 years, or 5 years). A skilled medical professional may determine the length of the treatment period using any of the methods described herein for diagnosing or following the effectiveness of treatment (e.g., the observation of at least one symptom of cancer). As described herein, a skilled medical professional can also change the identity and number (e.g., increase or decrease) of antibodies or antigen-binding antibody fragments, antibody-drug conjugates (and/or one or more additional therapeutic agents) administered to the subject and can also adjust (e.g., increase or decrease) the dosage or frequency of administration of at least one antibody or antigen-binding antibody fragment (and/or one or more additional therapeutic agents) to the subject based on an assessment of the effectiveness of the treatment (e.g., using any of the methods described herein and known in the art).

In some embodiments, one or more additional therapeutic agents can be administered to the subject. The additional therapeutic agent can comprise one or more inhibitors selected from the group consisting of an inhibitor of B-Raf, an EGFR inhibitor, an inhibitor of a MEK, an inhibitor of ERK, an inhibitor of K-Ras, an inhibitor of c-Met, an inhibitor of anaplastic lymphoma kinase (ALK), an inhibitor of a phosphatidylinositol 3-kinase (PI3K), an inhibitor of an Akt, an inhibitor of mTOR, a dual PI3K/mTOR inhibitor, an inhibitor of Bruton's tyrosine kinase (BTK), and an inhibitor of Isocitrate dehydrogenase 1 (IDH1) and/or Isocitrate dehydrogenase 2 (IDH2). In some embodiments, the additional therapeutic agent is an inhibitor of indoleamine 2,3-dioxygenase-1) (IDO1) (e.g., epacadostat).

In some embodiments, the additional therapeutic agent can comprise one or more inhibitors selected from the group consisting of an inhibitor of HER3, an inhibitor of LSD1, an inhibitor of MDM2, an inhibitor of BCL2, an inhibitor of CHK1, an inhibitor of activated hedgehog signaling pathway, and an agent that selectively degrades the estrogen receptor.

In some embodiments, the additional therapeutic agent can comprise one or more therapeutic agents selected from the group consisting of Trabectedin, nab-paclitaxel, Trebananib, Pazopanib, Cediranib, Palbociclib, everolimus, fluoropyrimidine, IFL, regorafenib, Reolysin, Alimta, Zykadia, Sutent, temsirolimus, axitinib, everolimus, sorafenib, Votrient, Pazopanib, IMA-901, AGS-003, cabozantinib, Vinflunine, an Hsp90 inhibitor, Ad-GM-CSF, Temazolomide, IL-2, IFNa, vinblastine, Thalomid, dacarbazine, cyclophosphamide, lenalidomide, azacytidine, lenalidomide, bortezomid, amrubicine, carfilzomib, pralatrexate, and enzastaurin.

In some embodiments, the additional therapeutic agent can comprise one or more therapeutic agents selected from the group consisting of an adjuvant, a TLR agonist, tumor necrosis factor (TNF) alpha, IL-1, HMGB1, an IL-10 antagonist, an IL-4 antagonist, an IL-13 antagonist, an IL-17 antagonist, an HVEM antagonist, an ICOS agonist, a treatment targeting CX3CL1, a treatment targeting CXCL9, a treatment targeting CXCL10, a treatment targeting CCL5, an LFA-1 agonist, an ICAM1 agonist, and a Selectin agonist.

In some embodiments, carboplatin, nab-paclitaxel, paclitaxel, cisplatin, pemetrexed, gemcitabine, FOLFOX, or FOLFIRI are administered to the subject.

In some embodiments, the additional therapeutic agent is an anti-OX40 antibody, an anti-PD-1 antibody, an anti-PD-L1 antibody, an anti-PD-L2 antibody, an anti-LAG-3 antibody, an anti-TIGIT antibody, an anti-BTLA antibody, an anti-CTLA-4 antibody, or an anti-GITR antibody.

### Pharmaceutical Compositions and Routes of Administration

Also provided herein are pharmaceutical compositions that contain at least one (e.g., one, two, three, or four) of the antibodies, antigen-binding fragments, or antibody-drug conjugates described herein. Two or more (e.g., two, three, or four) of any of the antibodies, antigen-binding fragments, or antibody-drug conjugates described herein can be present in a pharmaceutical composition in any combination. The pharmaceutical compositions may be formulated in any manner known in the art.

Pharmaceutical compositions are formulated to be compatible with their intended route of administration (e.g., intravenous, intraarterial, intramuscular, intradermal, subcutaneous, or intraperitoneal). The compositions can include a sterile diluent (e.g., sterile water or saline), a fixed oil, polyethylene glycol, glycerine, propylene glycol or other synthetic solvents, antibacterial or antifungal agents, such as benzyl alcohol or methyl parabens, chlorobutanol, phenol, ascorbic acid, thimerosal, and the like, antioxidants, such as ascorbic acid or sodium bisulfite, chelating agents, such as ethylenediaminetetraacetic acid, buffers, such as acetates, citrates, or phosphates, and isotonic agents, such as sugars (e.g., dextrose), polyalcohols (e.g., mannitol or sorbitol), or salts (e.g., sodium chloride), or any combination thereof. Liposomal suspensions can also be used as pharmaceutically acceptable carriers (see, e.g., U.S. Pat. No. 4,522,811). Preparations of the compositions can be formulated and enclosed in ampules, disposable syringes, or multiple dose vials. Where required (as in, for example, injectable formulations), proper fluidity can be maintained by, for example, the use of a coating, such as lecithin, or a surfactant. Absorption of the antibody or antigen-binding fragment thereof can be prolonged by including an agent that delays absorption (e.g., aluminum monostearate and gelatin). Alternatively, controlled release can be achieved by implants and microencapsulated delivery systems, which can include biodegradable, biocompatible polymers (e.g., ethylene vinyl acetate, polyanhydrides, polyglycolic acid, collagen, polyorthoesters, and polylactic acid; Alza Corporation and Nova Pharmaceutical, Inc.).

Compositions containing one or more of any of the antibodies, antigen-binding fragments, antibody-drug conjugates described herein can be formulated for parenteral (e.g., intravenous, intraarterial, intramuscular, intradermal, subcutaneous, or intraperitoneal) administration in dosage unit form (i.e., physically discrete units containing a predetermined quantity of active compound for ease of administration and uniformity of dosage).

Toxicity and therapeutic efficacy of compositions can be determined by standard pharmaceutical procedures in cell cultures or experimental animals (e.g., monkeys). One can determine the LD50 (the dose lethal to 50% of the population) and the ED50 (the dose therapeutically effective in 50% of the population): the therapeutic index being the ratio of LD50:ED50. Agents that exhibit high therapeutic indices are preferred. Where an agent exhibits an undesirable side effect, care should be taken to minimize potential damage (i.e., reduce unwanted side effects). Toxicity and therapeutic efficacy can be determined by other standard pharmaceutical procedures.

Data obtained from cell culture assays and animal studies can be used in formulating an appropriate dosage of any given agent for use in a subject (e.g., a human). A therapeutically effective amount of the one or more (e.g., one, two, three, or four) antibodies or antigen-binding fragments thereof (e.g., any of the antibodies or antibody fragments described herein) will be an amount that treats the disease in a subject (e.g., kills cancer cells) in a subject (e.g., a human subject identified as having cancer), or a subject identified as being at risk of developing the disease (e.g., a subject who has previously developed cancer but now has been cured), decreases the severity, frequency, and/or duration of one or more symptoms of a disease in a subject (e.g., a human). The effectiveness and dosing of any of the antibodies or antigen-binding fragments described herein can be determined by a health care professional or veterinary professional using methods known in the art, as well as by the observation of one or more symptoms of disease in a subject (e.g., a human). Certain factors may influence the dosage and timing required to effectively treat a subject (e.g., the severity of the disease or disorder, previous treatments, the general health and/or age of the subject, and the presence of other diseases).

Exemplary doses include milligram or microgram amounts of any of the antibodies or antigen-binding fragments, or antibody-drug conjugates described herein per kilogram of the subject's weight (e.g., about 1 μg/kg to about 500 mg/kg; about 100 μg/kg to about 500 mg/kg; about 100 μg/kg to about 50 mg/kg; about 10 μg/kg to about 5 mg/kg; about 10 μg/kg to about 0.5 mg/kg; or about 1 μg/kg to about 50 μg/kg). While these doses cover a broad range, one of ordinary skill in the art will understand that therapeutic agents, including antibodies and antigen-binding fragments thereof, vary in their potency, and effective amounts can be determined by methods known in the art. Typically, relatively low doses are administered at first, and the attending health care professional or veterinary professional (in the case of therapeutic application) or a researcher (when still working at the development stage) can subsequently and gradually increase the dose until an appropriate response is obtained. In addition, it is understood that the specific dose level for any particular subject will depend upon a variety of factors including the activity of the specific compound employed, the age, body weight, general health, gender, and diet of the subject, the time of administration, the route of administration, the rate of excretion, and the half-life of the antibody or antibody fragment in vivo.

The pharmaceutical compositions can be included in a container, pack, or dispenser together with instructions for administration. The disclosure also provides methods of manufacturing the antibodies or antigen binding fragments thereof, or antibody-drug conjugates for various uses as described herein.

### EXAMPLES

The invention is further described in the following examples, which do not limit the scope of the invention described in the claims.

### Example 1. BCMA/CD3 Antibody Expression and Purification

The plasmids encoding the VHH-Fc/VHH-VHH-Fc, together with plasmids encoding CD3 half IgG/CD3 ScFv-Fc were co-transfected into Expi293 cells using the ExpiFectamine 293 Transfection Kit (Thermo Fisher). Exemplary structure of VHH-Fc can be found, e.g., in FIG. 5A (left half structure). Exemplary structure of VHH-VHH-Fc can be found, e.g., in FIG. 5B (left half structure). Exemplary structure of CD3 half IgG can be found, e.g., in FIG. 5A (right half structure). Exemplary structure of CD3 ScFv-Fc can be found, e.g., in FIG. 6A (right half structure).

After 17 hours of transfection, enhancers were added to cells. The cells were lysed after 72 hours. The cell lysate containing the antibody was centrifuged at 3000 g for 10 minutes, and the supernatant was filtered with a 0.45 μm membrane. The antibody concentration was measured using a Protein A probe on Gator (Probe Life). Then, the antibody was purified using a Protein A column on AKTA Explorer 100 purification system (Buffer A: PBS, pH-7.4; Buffer B: 0.1 M glycine, pH 2.5), and then dialyzed twice in phosphate-buffered saline (PBS). The purified antibody was subjected to cation exchange chromatography (POROS GoPure HS Pre-packed Column, Thermo Fisher), with a salt gradient (Buffer A: 20 mM phosphate buffer, pH 7.4; Buffer B: 20 mM phosphate buffer, 1 M NaCl, pH 7.4), to enhance the purity of the preparation. Then, the antibody was dialyzed twice again in PBS, filtered with a 0.22 μm membrane, and tested by T-cell activation assay. The endotoxin content of the preparation was quantified using the Pierce LAL Chromogenic Endotoxin Quantitation Kit, and endotoxins were removed using the Pierce High Capacity Endotoxin Removal Spin Columns, according to manufacturer's instructions. Afterwards, the antibody was filtered again with a 0.22 μm membrane prior to usage.

### Example 2. Cell Binding Assays of Purified BCMA/CD3 Bispecific or Trispecific Antibodies

Binding of BCMA/CD3 bispecific or trispecific antibodies to CD3 and CD20 expressing cells was assessed by flow cytometry. Briefly, 2×10⁵ Jurkat (CD3+/CD20−) or Raji (CD20+/CD3−) cells resuspended in PBS with 2% bovine serum albumin (BSA) were incubated for 1 hour at room temperature with serial dilutions of BCMA/CD3 BsAbs which were diluted in PBS with 2% BSA. Then, the cells were washed twice with PBS (supplemented with BSA), and treated with PE-conjugated goat anti-human IgG (Jackson ImmunoResearch) for 30 minutes at 1:1000 dilution. Cells were washed again twice with PBS (supplemented with 2% BSA). The percentage of PE stained cells was measured using a FACSCalibur cytometer (BD Biosciences). Cell populations were visualized as forward vs side scatter and gated to exclude dead cells. Cells treated with no antibodies was used to establish background fluorescence.

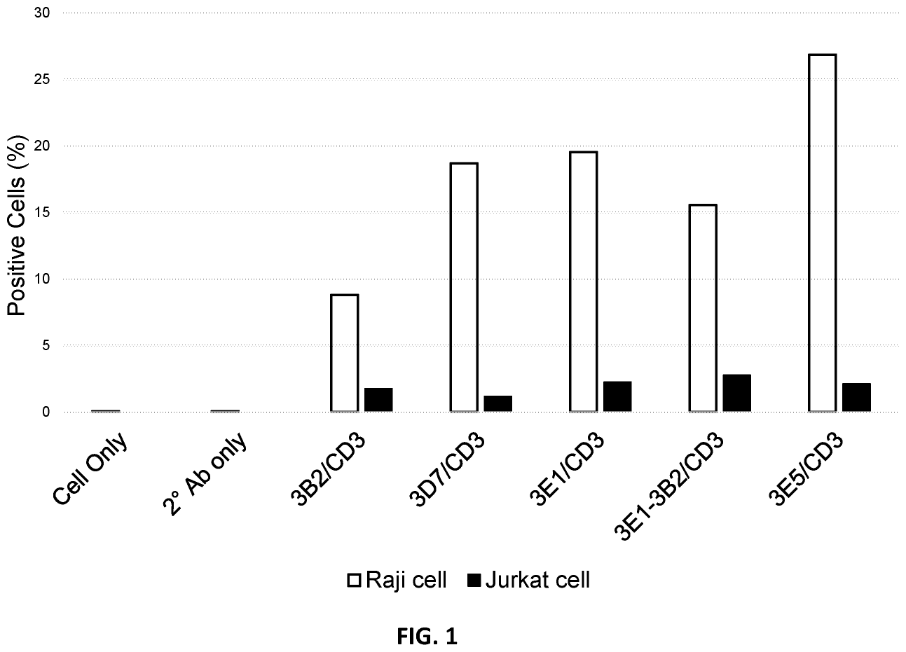

The bispecific antibodies 3B2/CD3, 3D7/CD3, 3E1/CD3, 3E5/CD3 with the BiSpecific-V1 structure (schematic structure shown in FIG. 5A), or trispecific antibody 3E1-3B2/CD3 with the TriSpecific-V1 structure (schematic structure shown in FIG. 5B) were incubated with Raji and Jurkat cells, respectively. The bound antibodies were detected by a PE-conjugated anti-Fc secondary antibody. As shown in FIG. 1, the tested bispecific or trispecific antibodies exhibited specific binding to both Raji cells and Jurkat cells. In particular, Raji cells showed much more positive cells than Jurkat cells, indicating the tested antibodies have a higher binding affinity to BCMA as compared to CD3.

### Example 3. T Cell Activation Assays for BCMA/CD3 Bispecific or Trispecific Antibodies

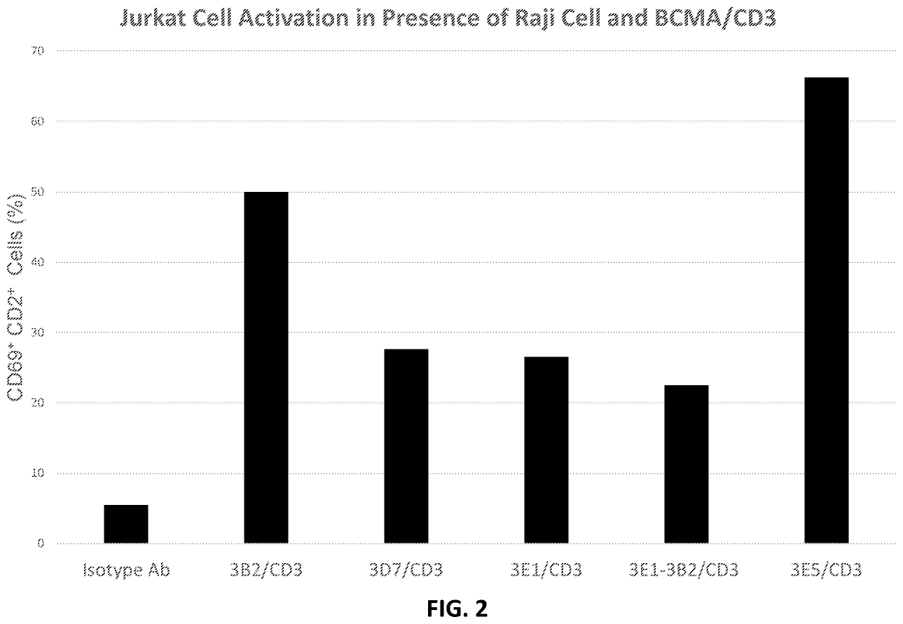

A fluorescence-activated cell sorting (FACS)-based approach was used to determine the ability of the BCMA/CD3 bispecific antibodies 3B2/CD3, 3D7/CD3, 3E1/CD3, 3E5/CD3 or trispecific antibody 3E1-3B2/CD3, to mediate the activation of the Jurkat cells, which is a CD3+ T cell lymphoblast-like cell line (ATCC TIB-152™). An amount of 2×10⁵ of Jurkat cells were co-incubated with CD20+ Raji cells at 1:1 for cell ratio for 16 hours with 10 μg/ml of indicated antibodies. The T cell activation was assessed by CD69+/CD2+ cell percentage by FACS following the treatment of cells with PE-conjugated anti-CD69 antibody and APC-conjugated anti-CD2 antibody. As shown in FIG. 2, the tested bispecific or trispecific antibodies induced Jurket cell activation in the presence of Raji cells.

### Example 4. T Cell Activation Assays for CD38/CD3 Bispecific Antibodies

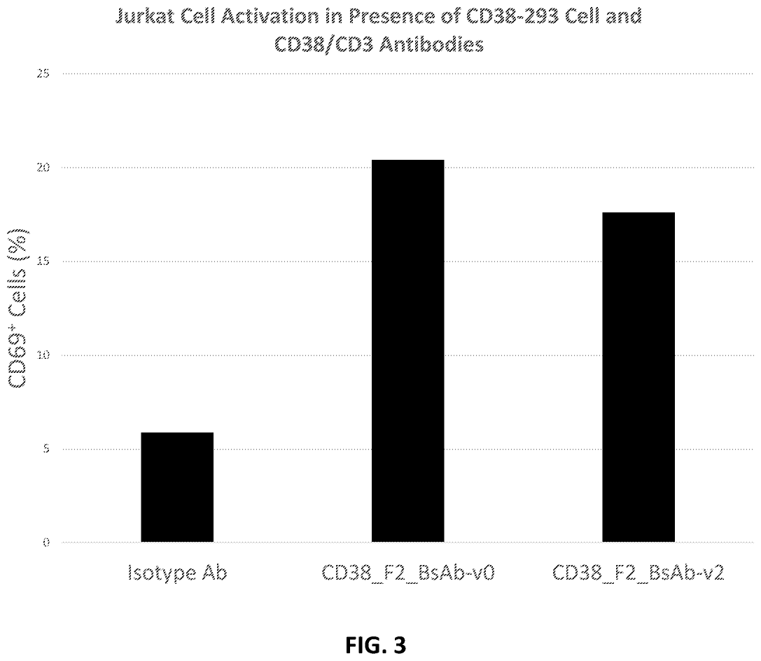

A fluorescence-activated cell sorting (FACS)-based approach was used to determine the ability of CD38/CD3 antibodies with different formats to mediate the activation of the Jurkat cells, in the presence of CD38+ target cells. The tested CD38/CD3 antibodies included a CD38-F2/CD3 bispecific antibody CD38-F2/BsAb-v0 with the BiSpecific-V0 structure (schematic structure shown in FIG. 9A) and a CD38-F2/CD3 bispecific antibody CD38-F2-BsAb-v2 with the BiSpecific-V2 structure (schematic structure shown in FIG. 11A). An amount of 2×10⁵ of Jurkat cells were co-incubated with CD38-expressing 293 cells at 1:1 for cell ratio for 24 hours with 8 μg/ml of the antibodies described herein. The T cell activation was assessed by CD69+ cell percentage by FACS. As shown in FIG. 3, both the tested CD38-F2/CD3 bispecific antibodies induced Jurket cell activation in the presence of CD38-expressing 293 cells.

### Example 5. Cell Binding Assays of CD38/CD3, BCMA/CD3 Bispecific Antibodies and a CD38/BCMA/CD3 Trispecific Antibody

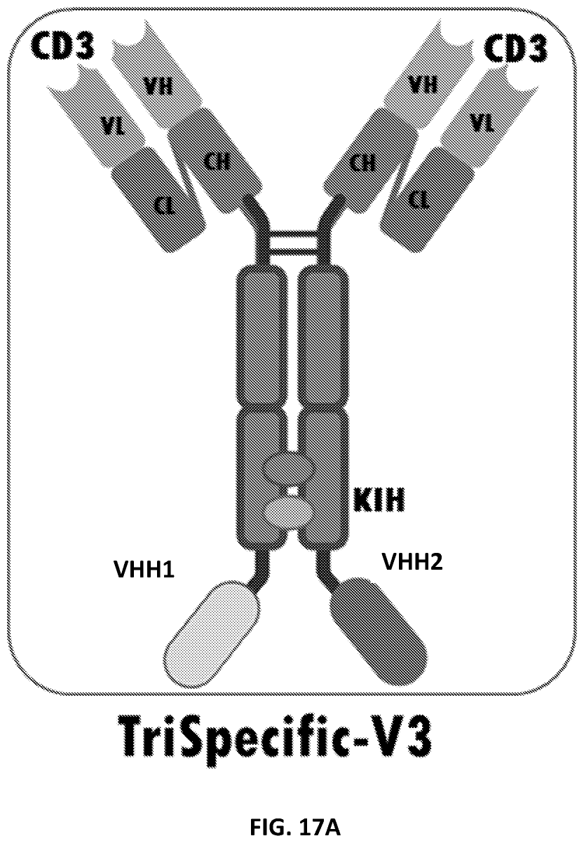

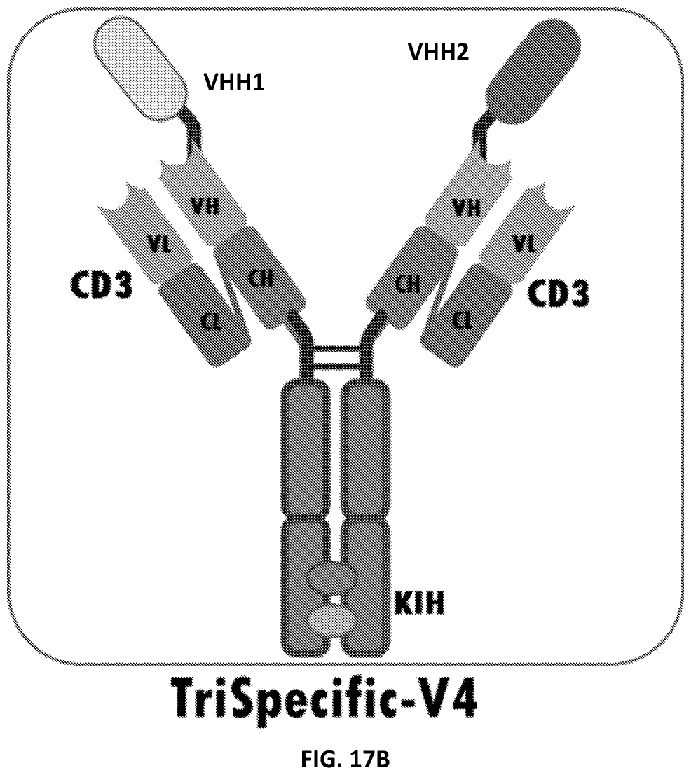

The binding affinity of a CD38/CD3 bispecific antibody (schematic structure shown in FIG. 13A, a BCMA/CD3 bispecific antibody (schematic structure shown in FIG. 8A), and a CD38/BCMA/CD3 trispecific antibody (schematic structure shown in FIG. 17B) to HCl-H929 cells (CD38+BCMA+), Jurkat cells (CD3+), Raji cells (CD38+), or 293 cells (CD38-BCMA-CD3−) were evaluated as follows.

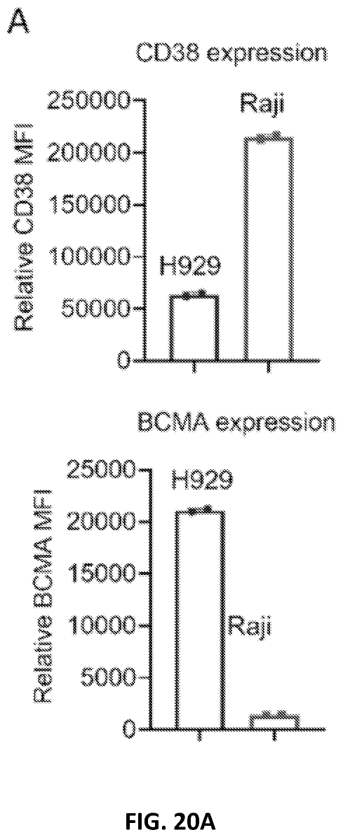

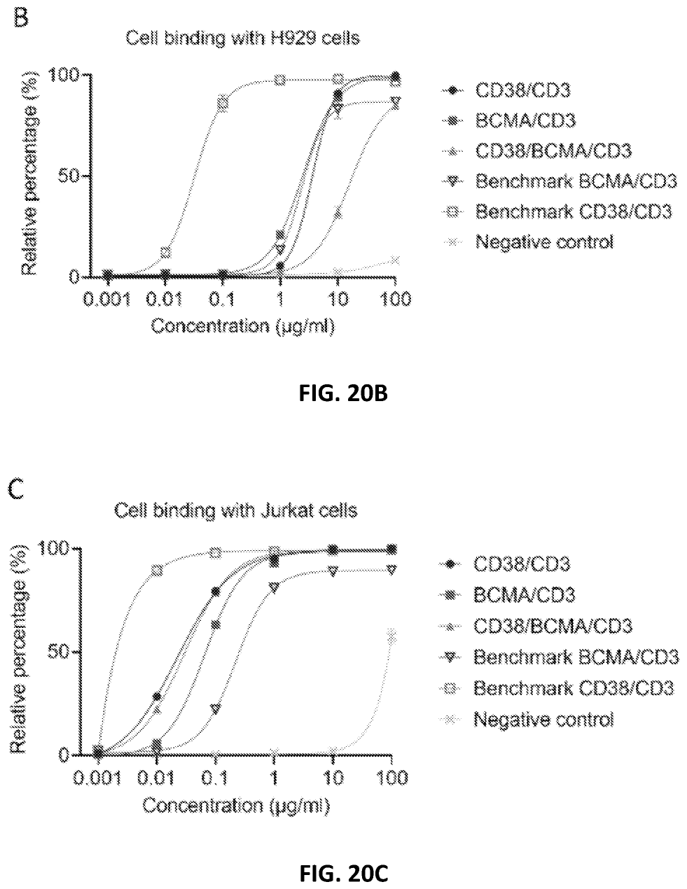

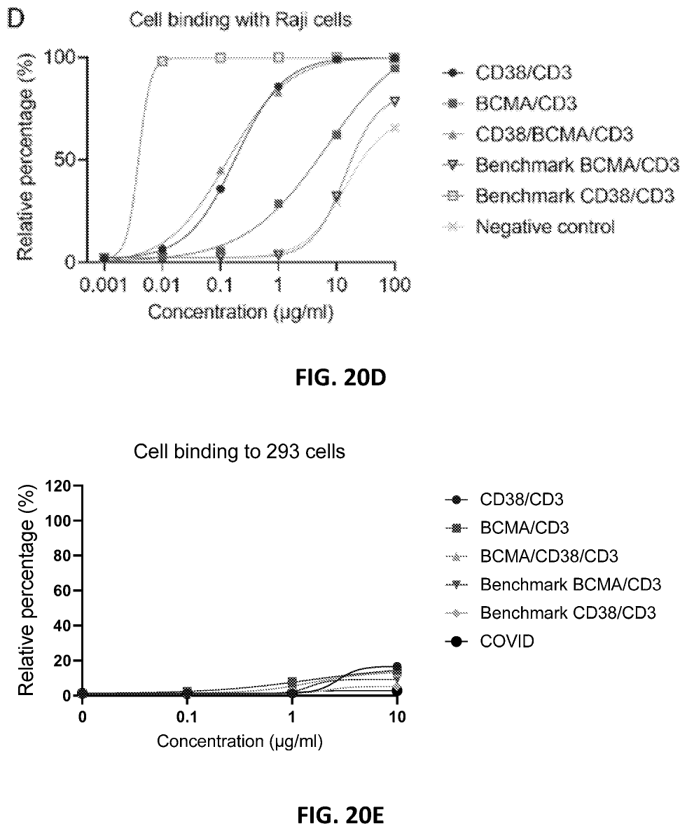

The expression levels of CD38 and BCMA in HCl-H929 and Raji cells were determined. Specifically, the suspension cell lines Raji and HCl-H929 were counted, and 5×10⁴ cells were washed twice with FACS buffer. The cells were resuspended in 60 μl of FACS buffer containing CD38-PE (1:100 dilution) or BCMA-PE (1:100 dilution), and incubated for 45 minutes at room temperature. Afterwards, the cells were washed twice with FACS buffer and resuspended in 80 μl FACS buffer for FACS analysis using a NovoCyte® cytometer. As shown in FIG. 20A, Raji cells expressed a high level of CD38 whereas HCl-H929 cells expressed a high level of BCMA. The result also showed that HCl-H929 cells expressed a relative low level of CD38, but Raji cells did not express BCMA.

Next, the antibody binding affinity to HCl-H929, Jurkat, Raji, and 293 cells were determined. Specifically, the cells were counted, and 5×10⁴ cells were washed twice with FACS buffer. The cells were resuspended in 60 μl of FACS buffer containing indicated concentrations of the antibodies and incubated for 1 hour at room temperature. After the incubation, the cells were washed twice with FACS buffer and resuspended in 100 μl of FACS buffer containing FITC-goat anti-human IgG Fc (1:500) and incubated for 30 minutes at room temperature. Then the cells were washed twice with FACS buffer and resuspended in 80 μl FACS buffer for FACS analysis using a NovoCyte® cytometer. The binding curves of each antibody against HCl-H929, Jurkat, Raji, and 293 cells are shown in FIGS. 20B-20E, respectively. The results showed that the CD38/CD3, BCMA/CD3, and CD38/BCMA/CD3 antibodies can bind to HCl-H929, Jurkat, and Raji cells. However, no binding was detected using 293 cells.

### Example 6. T Cell Activation Assays for CD38/CD3, BCMA/CD3 Bispecific Antibodies and a CD38/BCMA/CD3 Trispecific Antibody

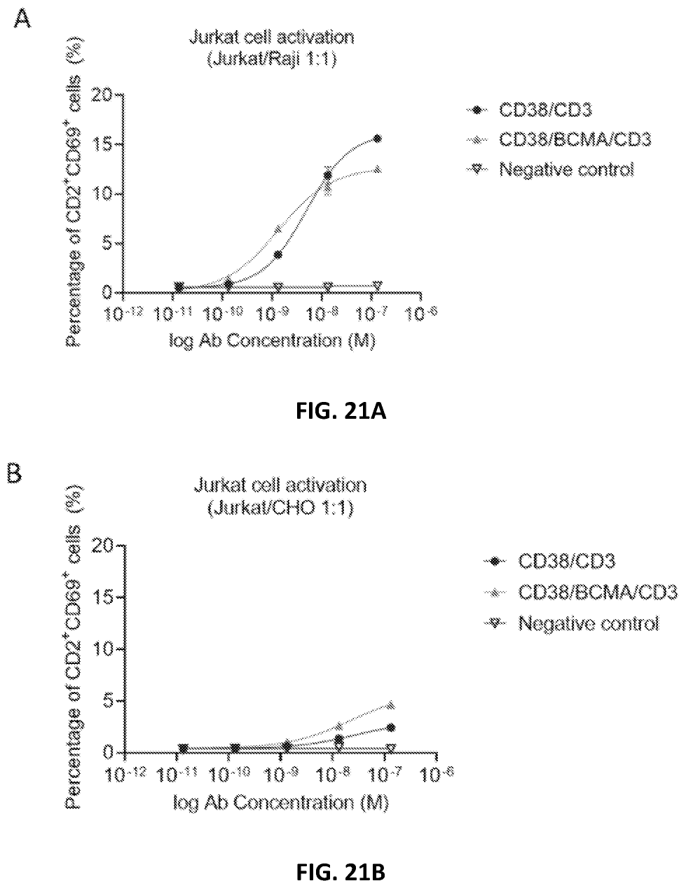

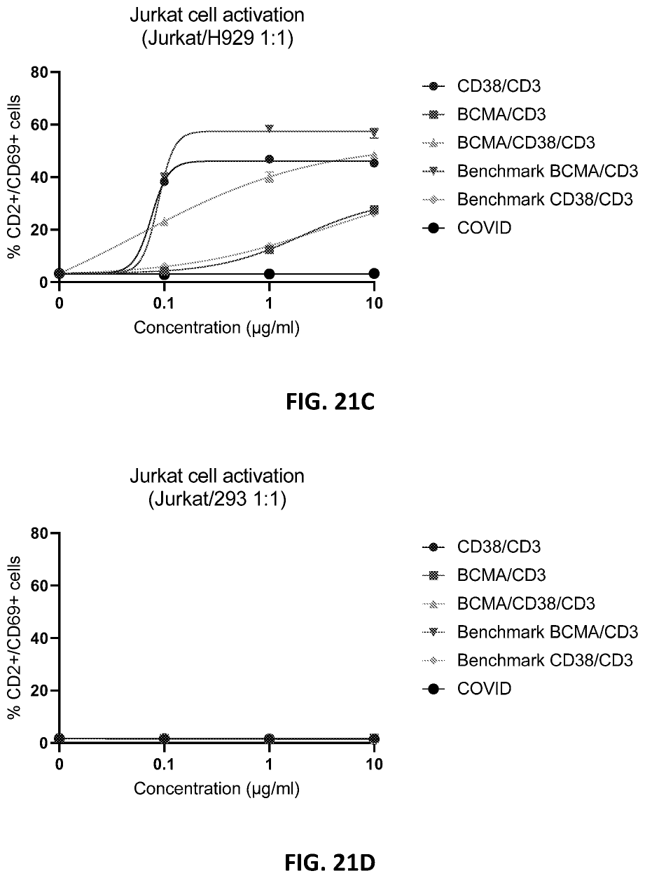

The effect of a CD38/CD3 bispecific antibody (schematic structure shown in FIG. 13A), a BCMA/CD3 bispecific antibody (schematic structure shown in FIG. 8A), and a CD38/BCMA/CD3 trispecific antibody (schematic structure shown in FIG. 17B) on T cell activation was compared. Specifically, Raji, CHO, HCl-H929, or 293 cells were mixed with Jurkat cell at a 1:1 ratio and added to a 96-well flat-bottom plate (2.5×10⁴ cells/well). The cells were treated with indicated concentrations of the antibodies, and co-cultured in a CO₂ incubator overnight. Afterwards, the cells were washed twice with FACS buffer. The cells were resuspended in 60 μl of FACS buffer containing CD2-APC (1:100 dilution) and CD69-PE (1:100 dilution), followed by a 30-minute incubation at room temperature. Then, the cells were washed twice with FACS buffer and resuspended in 80 μl FACS buffer for FACS analysis using a NovoCyte® cytometer. The activation curves of Jurkat cells mixed with Raji, CHO, HCl-H929, and 293 cells are shown in FIGS. 21A-21D, respectively. The results showed that the CD38/CD3, BCMA/CD3, and CD38/BCMA/CD3 antibodies can activate Jurkat cells in the presence of Raji and HCl-H929 cells. However, no Jurkat cell activation was detected using CHO or 293 cells.

### Example 7. Cancer Cell Killing and T Cell Activation Assays for CD38/CD3, BCMA/CD3 Bispecific Antibodies and a CD38/BCMA/CD3 Trispecific Antibody

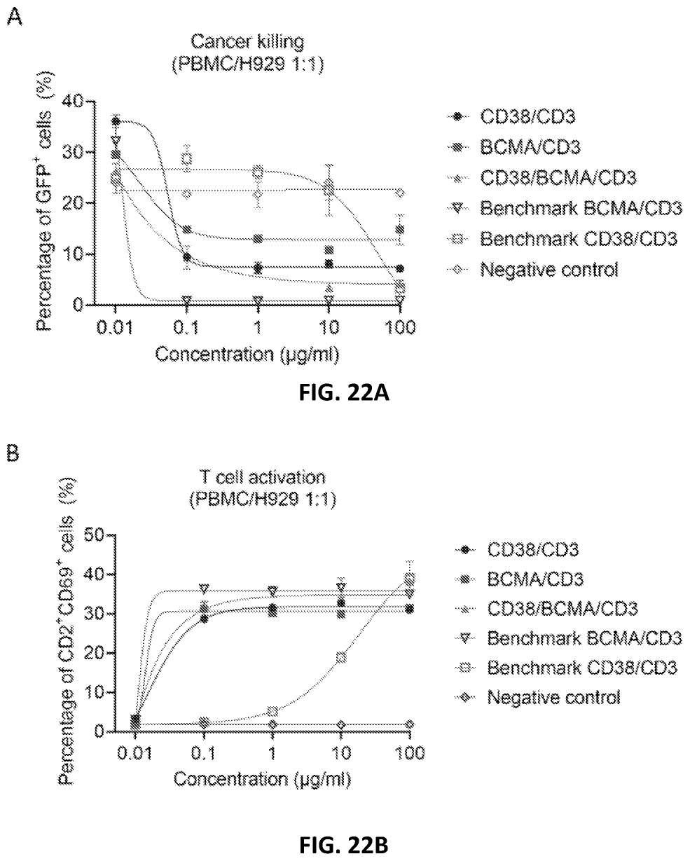

The effect of a CD38/CD3 bispecific antibody (schematic structure shown in FIG. 13A), a BCMA/CD3 bispecific antibody (schematic structure shown in FIG. 8A), and a CD38/BCMA/CD3 trispecific antibody (schematic structure shown in FIG. 17B) on cancer cell killing and T cell activation was determined as follows. Pre-labeled HCl-H929 cells were mixed with peripheral blood mononuclear cells (PBMCs) from a healthy donor at a 1:1 ratio and added to a 96-well flat-bottom plate (1×10⁵ cells/well). The cells were treated with indicated concentrations of the antibodies, and co-cultured in a CO₂ incubator for 48 hours. Afterwards, the cells were washed twice with FACS buffer. The cells were resuspended in 60 μl of FACS buffer containing CD2-APC (1:100 dilution) and CD69-PE (1:100 dilution), followed by a 30-minute incubation at room temperature. Then, the cells were washed twice with FACS buffer and resuspended in 80 μl FACS buffer containing 7-AAD (1:100). After 5 minutes at room temperature, the cells were analyzed by FACS using a NovoCyte© cytometer. The H292 cell killing curves are shown in FIG. 22A, and the T cell activation curves are shown in FIG. 22B. The results showed that the CD38/CD3, BCMA/CD3, and CD38/BCMA/CD3 antibodies exhibited better effects on cancer cell killing than a benchmark CD38/CD3 bispecific antibody, and similar T cell activation as compared to both benchmark CD38/CD3 (GBR1342) and BCMA/CD3 (REGN5458) bispecific antibodies.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. An antibody or antigen-binding fragment thereof that binds to BCMA (B-cell maturation antigen), comprising:
a heavy-chain antibody variable domain (VHH) comprising complementarity determining regions (CDRs) 1, 2, and 3, wherein the VHH CDR1 region comprises an amino acid sequence that is at least 80% identical to a selected VHH CDR1 amino acid sequence, the VHH CDR2 region comprises an amino acid sequence that is at least 80% identical to a selected VHH CDR2 amino acid sequence, and the VHH CDR3 region comprises an amino acid sequence that is at least 80% identical to a selected VHH CDR3 amino acid sequence;
wherein the selected VHH CDRs 1, 2, and 3 amino acid sequences are one of the following:
(1) the selected VHH CDRs 1, 2, 3 amino acid sequences are set forth in SEQ ID NOs: 1, 2, and 3, respectively;
(2) the selected VHH CDRs 1, 2, 3 amino acid sequences are set forth in SEQ ID NOs: 4, 5, and 6, respectively;
(3) the selected VHH CDRs 1, 2, 3 amino acid sequences are set forth in SEQ ID NOs: 7, 8, and 9, respectively; and
(4) the selected VHH CDRs 1, 2, 3 amino acid sequences are set forth in SEQ ID NOs: 10, 11, and 12, respectively.

2. The antibody or antigen-binding fragment thereof of claim 1, wherein the VHH comprises CDRs 1, 2, 3 with the amino acid sequences set forth in SEQ ID NOs: 1, 2, and 3, respectively.

3. The antibody or antigen-binding fragment thereof of claim 1, wherein the VHH comprises CDRs 1, 2, 3 with the amino acid sequences set forth in SEQ ID NOs: 4, 5, and 6, respectively.

4. The antibody or antigen-binding fragment thereof of claim 1, wherein the VHH comprises CDRs 1, 2, 3 with the amino acid sequences set forth in SEQ ID NOs: 7, 8, and 9, respectively.

5. The antibody or antigen-binding fragment thereof of claim 1, wherein the VHH comprises CDRs 1, 2, 3 with the amino acid sequences set forth in SEQ ID NOs: 10, 11, and 12, respectively.

6. An antibody or antigen-binding fragment thereof that binds to BCMA comprising a heavy-chain antibody variable domain (VHH) comprising an amino acid sequence that is at least 80% identical to a selected VHH sequence, wherein the selected VHH sequence is selected from the group consisting of SEQ ID NOs: 46-49.

7. The antibody or antigen-binding fragment thereof of claim 6, wherein the VHH comprises the sequence of SEQ ID NO: 46.

8. The antibody or antigen-binding fragment thereof of claim 6, wherein the VHH comprises the sequence of SEQ ID NO: 47.

9. The antibody or antigen-binding fragment thereof of claim 6, wherein the VHH comprises the sequence of SEQ ID NO: 48.

10. The antibody or antigen-binding fragment thereof of claim 6, wherein the VHH comprises the sequence of SEQ ID NO: 49.

11. The antibody or antigen-binding fragment thereof of any one of claims 1-10, wherein the antibody or antigen-binding fragment specifically binds to BCMA.

12. The antibody or antigen-binding fragment thereof of any one of claims 1-11, wherein the antibody or antigen-binding fragment is a humanized antibody or antigen-binding fragment thereof.

13. An antibody or antigen-binding fragment thereof comprising the VHH CDRs 1, 2, 3, of the antibody or antigen-binding fragment thereof of any one of claims 1-12.

14. The antibody or antigen-binding fragment thereof of any one of claims 1-13, wherein the antibody or antigen-binding fragment comprises a human IgG Fc.

15. The antibody or antigen-binding fragment thereof of any one of claims 1-14, wherein the antibody or antigen-binding fragment comprises two or more heavy-chain antibody variable domains.

16. An antibody or antigen-binding fragment thereof that cross-competes with the antibody or antigen-binding fragment thereof of any one of claims 1-15.

17. An antibody or antigen-binding fragment thereof that binds to CD38 (cluster of differentiation 38), comprising:
a heavy-chain antibody variable domain (VHH) comprising complementarity determining regions (CDRs) 1, 2, and 3, wherein the VHH CDR1 region comprises an amino acid sequence that is at least 80% identical to SEQ ID NO: 13, the VHH CDR2 region comprises an amino acid sequence that is at least 80% identical to SEQ ID NO: 14, and the VHH CDR3 region comprises an amino acid sequence that is at least 80% identical to SEQ ID NO: 15.

18. An antibody or antigen-binding fragment thereof that binds to CD38 comprising a heavy-chain antibody variable domain (VHH) comprising an amino acid sequence that is at least 80% identical to SEQ ID NO: 50.

19. The antibody or antigen-binding fragment thereof of claim 17 or 18, wherein the antibody or antigen-binding fragment specifically binds to CD38.

20. The antibody or antigen-binding fragment thereof of any one of claims 17-19, wherein the antibody or antigen-binding fragment is a humanized antibody or antigen-binding fragment thereof.

21. An antibody or antigen-binding fragment thereof comprising the VHH CDRs 1, 2, 3, of the antibody or antigen-binding fragment thereof of any one of claims 17-20.

22. The antibody or antigen-binding fragment thereof of any one of claims 17-21, wherein the antibody or antigen-binding fragment comprises a human IgG Fc.

23. The antibody or antigen-binding fragment thereof of any one of claims 17-22, wherein the antibody or antigen-binding fragment comprises two or more heavy-chain antibody variable domains.

24. An antibody or antigen-binding fragment thereof that cross-competes with the antibody or antigen-binding fragment thereof of any one of claims 17-23.

25. An antibody or antigen-binding fragment thereof that binds to CD3 (cluster of differentiation 3), comprising:
a heavy-chain antibody variable domain (VHH) comprising complementarity determining regions (CDRs) 1, 2, and 3, wherein the VHH CDR1 region comprises an amino acid sequence that is at least 80% identical to a selected VHH CDR1 amino acid sequence, the VHH CDR2 region comprises an amino acid sequence that is at least 80% identical to a selected VHH CDR2 amino acid sequence, and the VHH CDR3 region comprises an amino acid sequence that is at least 80% identical to a selected VHH CDR3 amino acid sequence;
wherein the selected VHH CDRs 1, 2, and 3 amino acid sequences are one of the following:
(1) the selected VHH CDRs 1, 2, 3 amino acid sequences are set forth in SEQ ID NOs: 16, 17, and 18, respectively;
(2) the selected VHH CDRs 1, 2, 3 amino acid sequences are set forth in SEQ ID NOs: 19, 20, and 21, respectively; and
(3) the selected VHH CDRs 1, 2, 3 amino acid sequences are set forth in SEQ ID NOs: 22, 23, and 24, respectively.

26. The antibody or antigen-binding fragment thereof of claim 25, wherein the VHH comprises CDRs 1, 2, 3 with the amino acid sequences set forth in SEQ ID NOs: 16, 17, and 18, respectively.

27. The antibody or antigen-binding fragment thereof of claim 25, wherein the VHH comprises CDRs 1, 2, 3 with the amino acid sequences set forth in SEQ ID NOs: 19, 20, and 21, respectively.

28. The antibody or antigen-binding fragment thereof of claim 25, wherein the VHH comprises CDRs 1, 2, 3 with the amino acid sequences set forth in SEQ ID NOs: 22, 23, and 24, respectively.

29. An antibody or antigen-binding fragment thereof that binds to CD3 comprising a heavy-chain antibody variable domain (VHH) comprising an amino acid sequence that is at least 80% identical to a selected VHH sequence, wherein the selected VHH sequence is selected from the group consisting of SEQ ID NOs: 51-53.

30. The antibody or antigen-binding fragment thereof of claim 29, wherein the VHH comprises the sequence of SEQ ID NO: 51.

31. The antibody or antigen-binding fragment thereof of claim 29, wherein the VHH comprises the sequence of SEQ ID NO: 52.

32. The antibody or antigen-binding fragment thereof of claim 29, wherein the VHH comprises the sequence of SEQ ID NO: 53.

33. The antibody or antigen-binding fragment thereof of any one of claims 25-32, wherein the antibody or antigen-binding fragment specifically binds to CD3.

34. The antibody or antigen-binding fragment thereof of any one of claims 25-33, wherein the antibody or antigen-binding fragment is a humanized antibody or antigen-binding fragment thereof.

35. An antibody or antigen-binding fragment thereof comprising the VHH CDRs 1, 2, 3, of the antibody or antigen-binding fragment thereof of any one of claims 25-34.

36. The antibody or antigen-binding fragment thereof of any one of claims 25-35, wherein the antibody or antigen-binding fragment comprises a human IgG Fc.

37. The antibody or antigen-binding fragment thereof of any one of claims 25-36, wherein the antibody or antigen-binding fragment comprises two or more heavy-chain antibody variable domains.

38. An antibody or antigen-binding fragment thereof that cross-competes with the antibody or antigen-binding fragment thereof of any one of claims 25-37.

39. An antibody or antigen-binding fragment thereof that binds to CD3 (cluster of differentiation 3) comprising:
a heavy chain variable region (VH) comprising complementarity determining regions (CDRs) 1, 2, and 3, wherein the VH CDR1 region comprises an amino acid sequence that is at least 80% identical to a selected VH CDR1 amino acid sequence, the VH CDR2 region comprises an amino acid sequence that is at least 80% identical to a selected VH CDR2 amino acid sequence, and the VH CDR3 region comprises an amino acid sequence that is at least 80% identical to a selected VH CDR3 amino acid sequence; and
a light chain variable region (VL) comprising CDRs 1, 2, and 3, wherein the VL CDR1 region comprises an amino acid sequence that is at least 80% identical to a selected VL CDR1 amino acid sequence, the VL CDR2 region comprises an amino acid sequence that is at least 80% identical to a selected VL CDR2 amino acid sequence, and the VL CDR3 region comprises an amino acid sequence that is at least 80% identical to a selected VL CDR3 amino acid sequence,
wherein the selected VH CDRs 1, 2, and 3 amino acid sequences and the selected VL CDRs, 1, 2, and 3 amino acid sequences are one of the following:
(1) the selected VH CDRs 1, 2, 3 amino acid sequences are set forth in SEQ ID NOs: 25, 26, and 27, respectively, and the selected VL CDRs 1, 2, 3 amino acid sequences are set forth in SEQ ID NOs: 28, 29, and 30, respectively;
(2) the selected VH CDRs 1, 2, 3 amino acid sequences are set forth in SEQ ID NOs: 25, 26, and 27, respectively, and the selected VL CDRs 1, 2, 3 amino acid sequences are set forth in SEQ ID NOs: 31, 32, and 33, respectively;
(3) the selected VH CDRs 1, 2, 3 amino acid sequences are set forth in SEQ ID NOs: 25, 26, and 27, respectively, and the selected VL CDRs 1, 2, 3 amino acid sequences are set forth in SEQ ID NOs: 34, 35, and 36, respectively;
(4) the selected VH CDRs 1, 2, 3 amino acid sequences are set forth in SEQ ID NOs: 25, 26, and 27, respectively, and the selected VL CDRs 1, 2, 3 amino acid sequences are set forth in SEQ ID NOs: 37, 38, and 39, respectively;
(5) the selected VH CDRs 1, 2, 3 amino acid sequences are set forth in SEQ ID NOs: 25, 26, and 27, respectively, and the selected VL CDRs 1, 2, 3 amino acid sequences are set forth in SEQ ID NOs: 40, 41, and 42, respectively; and
(6) the selected VH CDRs 1, 2, 3 amino acid sequences are set forth in SEQ ID NOs: 25, 26, and 27, respectively, and the selected VL CDRs 1, 2, 3 amino acid sequences are set forth in SEQ ID NOs: 43, 44, and 45, respectively.

40. The antibody or antigen-binding fragment thereof of claim 39, wherein the VH comprises CDRs 1, 2, 3 with the amino acid sequences set forth in SEQ ID NOs: 25, 26, and 27, respectively, and the VL comprises CDRs 1, 2, 3 with the amino acid sequences set forth in SEQ ID NOs: 28, 29, and 30, respectively.

41. The antibody or antigen-binding fragment thereof of claim 39, wherein the VH comprises CDRs 1, 2, 3 with the amino acid sequences set forth in SEQ ID NOs: 25, 26, and 27, respectively, and the VL comprises CDRs 1, 2, 3 with the amino acid sequences set forth in SEQ ID NOs: 31, 32, and 33, respectively.

42. The antibody or antigen-binding fragment thereof of claim 39, wherein the VH comprises CDRs 1, 2, 3 with the amino acid sequences set forth in SEQ ID NOs: 25, 26, and 27, respectively, and the VL comprises CDRs 1, 2, 3 with the amino acid sequences set forth in SEQ ID NOs: 34, 35, and 36, respectively.

43. The antibody or antigen-binding fragment thereof of claim 39, wherein the VH comprises CDRs 1, 2, 3 with the amino acid sequences set forth in SEQ ID NOs: 25, 26, and 27, respectively, and the VL comprises CDRs 1, 2, 3 with the amino acid sequences set forth in SEQ ID NOs: 37, 38, and 39, respectively.

44. The antibody or antigen-binding fragment thereof of claim 39, wherein the VH comprises CDRs 1, 2, 3 with the amino acid sequences set forth in SEQ ID NOs: 25, 26, and 27, respectively, and the VL comprises CDRs 1, 2, 3 with the amino acid sequences set forth in SEQ ID NOs: 40, 41, and 42, respectively.

45. The antibody or antigen-binding fragment thereof of claim 39, wherein the VH comprises CDRs 1, 2, 3 with the amino acid sequences set forth in SEQ ID NOs: 25, 26, and 27, respectively, and the VL comprises CDRs 1, 2, 3 with the amino acid sequences set forth in SEQ ID NOs: 43, 44, and 45, respectively.

46. An antibody or antigen-binding fragment thereof that binds to CD3 comprising a heavy chain variable region (VH) comprising an amino acid sequence that is at least 90% identical to SEQ ID NO: 54, and a light chain variable region (VL) comprising an amino acid sequence that is at least 90% identical to SEQ ID NO: 55, 56, 57, 58, 59, or 60.

47. The antibody or antigen-binding fragment thereof of claim 50, wherein the VH comprises the sequence of SEQ ID NO: 54 and the VL comprises the sequence of SEQ ID NO: 55.

48. The antibody or antigen-binding fragment thereof of claim 50, wherein the VH comprises the sequence of SEQ ID NO: 54 and the VL comprises the sequence of SEQ ID NO: 56.

49. The antibody or antigen-binding fragment thereof of claim 50, wherein the VH comprises the sequence of SEQ ID NO: 54 and the VL comprises the sequence of SEQ ID NO: 57.

50. The antibody or antigen-binding fragment thereof of claim 50, wherein the VH comprises the sequence of SEQ ID NO: 54 and the VL comprises the sequence of SEQ ID NO: 58.

51. The antibody or antigen-binding fragment thereof of claim 50, wherein the VH comprises the sequence of SEQ ID NO: 54 and the VL comprises the sequence of SEQ ID NO: 59.

52. The antibody or antigen-binding fragment thereof of claim 50, wherein the VH comprises the sequence of SEQ ID NO: 54 and the VL comprises the sequence of SEQ ID NO: 60.

53. The antibody or antigen-binding fragment thereof of any one of claims 39-52, wherein the antibody or antigen-binding fragment specifically binds to human CD3.

54. The antibody or antigen-binding fragment thereof of any one of claims 39-53, wherein the antibody or antigen-binding fragment is a single-chain variable fragment (scFV).

55. An antibody or antigen-binding fragment thereof comprising the VH CDRs 1, 2, 3, and VL CDRs 1, 2, 3, of the antibody or antigen-binding fragment thereof of any one of claims 39-55.

56. An antibody or antigen-binding fragment thereof that cross-competes with the antibody or antigen-binding fragment thereof of any one of claims 39-55.

57. The antibody or antigen-binding fragment thereof of any one of claims 1-56, wherein the antibody is a bispecific antibody or a multispecific antibody.

58. A multi-specific antibody or antigen-binding fragment thereof, comprising a first antigen-binding site that specifically binds to CD3, and a second antigen-binding site that specifically binds to BCMA or CD38.

59. The multi-specific antibody or antigen-binding fragment thereof of claim 58, further comprising a third antigen-binding site that specifically binds to BCMA or CD38.

60. The multi-specific antibody or antigen-binding fragment thereof of claim 59, wherein
a) the second antigen-binding site that specifically binds to BCMA and the third antigen-binding site that specifically binds to BCMA;
b) the second antigen-binding site that specifically binds to BCMA and the third antigen-binding site that specifically binds to CD38; or
c) the second antigen-binding site that specifically binds to CD38 and the third antigen-binding site that specifically binds to CD38.

61. The multi-specific antibody or antigen-binding fragment thereof of anyone of claim 58-60, wherein the first antigen-binding site comprises a first heavy-chain antibody variable domain (VHH1) comprising complementarity determining regions (CDRs) 1, 2, and 3, wherein the VHH1 CDR1 region comprises an amino acid sequence that is at least 80% identical to a selected VHH1 CDR1 amino acid sequence, the VHH1 CDR2 region comprises an amino acid sequence that is at least 80% identical to a selected VHH1 CDR2 amino acid sequence, and the VHH1 CDR3 region comprises an amino acid sequence that is at least 80% identical to a selected VHH1 CDR3 amino acid sequence;
wherein the selected VHH1 CDRs 1, 2, and 3 amino acid sequences are one of the following:
(1) the selected VHH1 CDRs 1, 2, 3 amino acid sequences are set forth in SEQ ID NOs: 16, 17, and 18, respectively;
(2) the selected VHH1 CDRs 1, 2, 3 amino acid sequences are set forth in SEQ ID NOs: 19, 20, and 21, respectively; and
(3) the selected VHH1 CDRs 1, 2, 3 amino acid sequences are set forth in SEQ ID NOs: 22, 23, and 24, respectively.

62. The multi-specific antibody or antigen-binding fragment thereof of claim 61, wherein the first heavy-chain antibody variable domain (VHH1) comprises an amino acid sequence that is at least 80% identical to a selected VHH1 sequence, wherein the selected VHH1 sequence is selected from the group consisting of SEQ ID NOs: 51-53.

63. The multi-specific antibody or antigen-binding fragment thereof of any one of claims 59-62, wherein the second antigen-binding site specifically binds to BCMA, and the second antigen-binding site comprises a second heavy-chain antibody variable domain (VHH2) comprising complementarity determining regions (CDRs) 1, 2, and 3, wherein the VHH2 CDR1 region comprises an amino acid sequence that is at least 80% identical to a selected VHH2 CDR1 amino acid sequence, the VHH2 CDR2 region comprises an amino acid sequence that is at least 80% identical to a selected VHH2 CDR2 amino acid sequence, and the VHH2 CDR3 region comprises an amino acid sequence that is at least 80% identical to a selected VHH2 CDR3 amino acid sequence;
wherein the selected VHH2 CDRs 1, 2, and 3 amino acid sequences are one of the following:
(1) the selected VHH2 CDRs 1, 2, 3 amino acid sequences are set forth in SEQ ID NOs: 1, 2, and 3, respectively;
(2) the selected VHH2 CDRs 1, 2, 3 amino acid sequences are set forth in SEQ ID NOs: 4, 5, and 6, respectively;
(3) the selected VHH2 CDRs 1, 2, 3 amino acid sequences are set forth in SEQ ID NOs: 7, 8, and 9, respectively; and
(4) the selected VHH2 CDRs 1, 2, 3 amino acid sequences are set forth in SEQ ID NOs: 10, 11, and 12, respectively.

64. The multi-specific antibody or antigen-binding fragment thereof of claim 63, wherein the second heavy-chain antibody variable domain (VHH2) comprises an amino acid sequence that is at least 80% identical to a selected VHH2 sequence, wherein the selected VHH2 sequence is selected from the group consisting of SEQ ID NOs: 46-49.

65. The multi-specific antibody or antigen-binding fragment thereof of any one of claims 59-62, wherein the second antigen-binding site specifically binds to CD38, wherein the second antigen-binding site comprises a second heavy-chain antibody variable domain (VHH2) comprising complementarity determining regions (CDRs) 1, 2, and 3, wherein the VHH2 CDR1 region comprises an amino acid sequence that is at least 80% identical to SEQ ID NO: 13, the VHH2 CDR2 region comprises an amino acid sequence that is at least 80% identical to SEQ ID NO: 14, and the VHH2 CDR3 region comprises an amino acid sequence that is at least 80% identical to SEQ ID NO: 15.

66. The multi-specific antibody or antigen-binding fragment thereof of claim 65, wherein the second heavy-chain antibody variable domain (VHH2) comprises an amino acid sequence that is at least 80% identical to SEQ ID NO: 50.

67. The multi-specific antibody or antigen-binding fragment thereof of any one of claims 58-60, wherein the first antigen-binding site comprises a heavy chain variable region (VH) and a light chain variable region (VL), wherein the VH and the VL associate with each other and specifically bind to CD3, wherein
the heavy chain variable region (VH) comprising complementarity determining regions (CDRs) 1, 2, and 3, wherein the VH CDR1 region comprises an amino acid sequence that is at least 80% identical to a selected VH CDR1 amino acid sequence, the VH CDR2 region comprises an amino acid sequence that is at least 80% identical to a selected VH CDR2 amino acid sequence, and the VH CDR3 region comprises an amino acid sequence that is at least 80% identical to a selected VH CDR3 amino acid sequence; and
the light chain variable region (VL) comprising CDRs 1, 2, and 3, wherein the VL CDR1 region comprises an amino acid sequence that is at least 80% identical to a selected VL CDR1 amino acid sequence, the VL CDR2 region comprises an amino acid sequence that is at least 80% identical to a selected VL CDR2 amino acid sequence, and the VL CDR3 region comprises an amino acid sequence that is at least 80% identical to a selected VL CDR3 amino acid sequence,
wherein the selected VH CDRs 1, 2, and 3 amino acid sequences and the selected VL CDRs, 1, 2, and 3 amino acid sequences are one of the following:
(1) the selected VH CDRs 1, 2, 3 amino acid sequences are set forth in SEQ ID NOs: 25, 26, and 27, respectively, and the selected VL CDRs 1, 2, 3 amino acid sequences are set forth in SEQ ID NOs: 28, 29, and 30, respectively;
(2) the selected VH CDRs 1, 2, 3 amino acid sequences are set forth in SEQ ID NOs: 25, 26, and 27, respectively, and the selected VL CDRs 1, 2, 3 amino acid sequences are set forth in SEQ ID NOs: 31, 32, and 33, respectively;
(3) the selected VH CDRs 1, 2, 3 amino acid sequences are set forth in SEQ ID NOs: 25, 26, and 27, respectively, and the selected VL CDRs 1, 2, 3 amino acid sequences are set forth in SEQ ID NOs: 34, 35, and 36, respectively;
(4) the selected VH CDRs 1, 2, 3 amino acid sequences are set forth in SEQ ID NOs: 25, 26, and 27, respectively, and the selected VL CDRs 1, 2, 3 amino acid sequences are set forth in SEQ ID NOs: 37, 38, and 39, respectively;
(5) the selected VH CDRs 1, 2, 3 amino acid sequences are set forth in SEQ ID NOs: 25, 26, and 27, respectively, and the selected VL CDRs 1, 2, 3 amino acid sequences are set forth in SEQ ID NOs: 40, 41, and 42, respectively; and
(6) the selected VH CDRs 1, 2, 3 amino acid sequences are set forth in SEQ ID NOs: 25, 26, and 27, respectively, and the selected VL CDRs 1, 2, 3 amino acid sequences are set forth in SEQ ID NOs: 43, 44, and 45, respectively.

68. The multi-specific antibody or antigen-binding fragment thereof of claim 67, wherein the heavy chain variable region (VH) comprises an amino acid sequence that is at least 90% identical to SEQ ID NO: 54, and a light chain variable region (VL) comprising an amino acid sequence that is at least 90% identical to SEQ ID NO: 55, 56, 57, 58, 59, or 60.

69. The multi-specific antibody or antigen-binding fragment thereof of claim 67 or 68, wherein the second antigen-binding site specifically binds to BCMA, and the second antigen-binding site comprises a heavy-chain antibody variable domain (VHH) comprising complementarity determining regions (CDRs) 1, 2, and 3, wherein the VHH CDR1 region comprises an amino acid sequence that is at least 80% identical to a selected VHH CDR1 amino acid sequence, the VHH CDR2 region comprises an amino acid sequence that is at least 80% identical to a selected VHH CDR2 amino acid sequence, and the VHH CDR3 region comprises an amino acid sequence that is at least 80% identical to a selected VHH CDR3 amino acid sequence;
wherein the selected VHH CDRs 1, 2, and 3 amino acid sequences are one of the following:
(1) the selected VHH CDRs 1, 2, 3 amino acid sequences are set forth in SEQ ID NOs: 1, 2, and 3, respectively;
(2) the selected VHH CDRs 1, 2, 3 amino acid sequences are set forth in SEQ ID NOs: 4, 5, and 6, respectively;
(3) the selected VHH CDRs 1, 2, 3 amino acid sequences are set forth in SEQ ID NOs: 7, 8, and 9, respectively; and
(4) the selected VHH CDRs 1, 2, 3 amino acid sequences are set forth in SEQ ID NOs: 10, 11, and 12, respectively.

70. The multi-specific antibody or antigen-binding fragment thereof of claim 69, wherein the heavy-chain antibody variable domain (VHH) comprises an amino acid sequence that is at least 80% identical to a selected VHH sequence, wherein the selected VHH sequence is selected from the group consisting of SEQ ID NOs: 46-49.

71. The multi-specific antibody or antigen-binding fragment thereof of claim 67 or 68, wherein the second antigen-binding site specifically binds to CD38, wherein the second antigen-binding site comprises a heavy-chain antibody variable domain (VHH) comprising complementarity determining regions (CDRs) 1, 2, and 3, wherein the VHH CDR1 region comprises an amino acid sequence that is at least 80% identical to SEQ ID NO: 13, the VHH CDR2 region comprises an amino acid sequence that is at least 80% identical to SEQ ID NO: 14, and the VHH CDR3 region comprises an amino acid sequence that is at least 80% identical to SEQ ID NO: 15.

72. The multi-specific antibody or antigen-binding fragment thereof of claim 71, wherein the heavy-chain antibody variable domain (VHH) comprises an amino acid sequence that is at least 80% identical to SEQ ID NO: 50.

73. The multi-specific antibody or antigen-binding fragment thereof of any one of claims 67-72, wherein the VH and the VL are linked by a linker peptide sequence to form an scFv.

74. The multi-specific antibody or antigen-binding fragment thereof of claim 73, wherein the linker peptide sequence comprises a sequence that is at least 80% identical to any one of SEQ ID NOs: 71-73 and 124-126.

75. A polypeptide complex, comprising
(a) a first polypeptide comprising from N-terminus to C-terminus: a first heavy-chain antibody variable domain (VHH), a first hinge region, a first Fc region; and
(b) a second polypeptide comprising from N-terminus to C-terminus: a second VHH, a second hinge region, and a second Fc region;
wherein the first VHH specifically binds to BCMA or CD38, wherein the second VHH specifically binds to CD3.

76. The polypeptide complex of claim 75, wherein the first hinge region and/or the second hinge region comprise a sequence that is at least 80% identical to any one of SEQ ID NOs: 61-65.

77. The polypeptide complex of claim 75 or 76, wherein the first Fc region and/or the second Fc region comprise a sequence that is at least 80% identical to SEQ ID NO: 66 or 67.

78. The polypeptide complex of any one of claims 75-77, wherein the first polypeptide comprises a sequence that is at least 80% identical to SEQ ID NO: 74, 75, 116, or 117; and/or wherein the second polypeptide comprises a sequence that is at least 80% identical to SEQ ID NOs: 76, 77, 118, or 119.

79. The polypeptide complex of any one of claims 75-77, wherein the first polypeptide further comprises a third VHH that specifically binds to BCMA or CD38, wherein the third VHH is linked to the N-terminus of the first VHH via a linker peptide sequence.

80. The polypeptide complex of claim 79, wherein the linker peptide sequence comprises a sequence that is at least 80% identical to any one of SEQ ID NOs: 71-73 and 124-126.

81. The polypeptide complex of claim 79 or 80, wherein the first polypeptide comprises a sequence that is at least 80% identical to SEQ ID NO: 78 or 79; and/or wherein the second polypeptide comprises a sequence that is at least 80% identical to SEQ ID NO: 80 or 81.

82. A polypeptide complex, comprising
(a) a first polypeptide comprising from N-terminus to C-terminus: a first heavy-chain antibody variable domain (VHH), a first hinge region, a first Fc region;
(b) a second polypeptide comprising from N-terminus to C-terminus: a heavy chain variable region (VH), a second hinge region, and a second Fc region; and
(c) a third polypeptide comprising a light chain variable region (VL);
wherein the first VHH specifically binds to BCMA or CD38, wherein the VH and the VL associate with each other and specifically bind to CD3.

83. The polypeptide complex of claim 82, wherein the first hinge region and/or the second hinge region comprise a sequence that is at least 80% identical to any one of SEQ ID NOs: 61-65.

84. The polypeptide complex of claim 82 or 83, wherein the first Fc region and/or the second Fc region comprise a sequence that is at least 80% identical to SEQ ID NO: 66 or 67.

85. The polypeptide complex of any one of claims 82-84, wherein the first polypeptide comprises a sequence that is at least 80% identical to SEQ ID NO: 82 or 83; wherein the second polypeptide comprises a sequence that is at least 80% identical to SEQ ID NO: 84 or 85; and/or wherein the third polypeptide comprises a sequence that is at least 80% identical to SEQ ID NO: 86.

86. The polypeptide complex of any one of claims 82-84, wherein the first polypeptide further comprises a second VHH that specifically binds to BCMA or CD38, wherein the second VHH is linked to the N-terminus of the first VHH via a linker peptide sequence.

87. The polypeptide complex of claim 84, wherein the linker peptide sequence comprises a sequence that is at least 80% identical to any one of SEQ ID NOs: 71-73 and 124-126.

88. The polypeptide complex of claim 84 or 85, wherein the first polypeptide comprises a sequence that is at least 80% identical to SEQ ID NO: 87 or 88; wherein the second polypeptide comprises a sequence that is at least 80% identical to SEQ ID NO: 89 or 90; and/or wherein the third polypeptide comprises a sequence that is at least 80% identical to SEQ ID NO: 91.

89. A polypeptide complex, comprising
(a) a first polypeptide comprising from N-terminus to C-terminus: a first heavy-chain antibody variable domain (VHH), a first hinge region, a first Fc region; and
(b) a second polypeptide comprising from N-terminus to C-terminus: a single-chain variable fragment (scFv), a second hinge region, and a second Fc region;
wherein the first VHH specifically binds to BCMA or CD38; wherein the scFv comprises a heavy chain variable region (VH), a first linker peptide sequence, and a light chain variable region (VL); wherein the VH and the VL associate with each other and specifically bind to CD3.

90. The polypeptide complex of claim 89, wherein the first linker peptide sequence comprises a sequence that is at least 80% identical to any one of SEQ ID NOs: 71-73 and 124-126.

91. The polypeptide complex of claim 89 or 90, wherein the first hinge region and/or the second hinge region comprise a sequence that is at least 80% identical to any one of SEQ ID NOs: 61-65.

92. The polypeptide complex of any one of claims 89-91, wherein the first Fc region and/or the second Fc region comprise a sequence that is at least 80% identical to SEQ ID NO: 66 or 67.

93. The polypeptide complex of any one of claims 89-92, wherein the first polypeptide comprises a sequence that is at least 80% identical to SEQ ID NO: 92, 93, 120, or 121; and/or wherein the second polypeptide comprises a sequence that is at least 80% identical to SEQ ID NO: 94, 95, 122, or 123.

94. The polypeptide complex of any one of claims 89-92, wherein the first polypeptide further comprises a second VHH that specifically binds to BCMA or CD38, wherein the second VHH is linked to the N-terminus of the first VHH via a second linker peptide sequence.

95. The polypeptide complex of claim 94, wherein the second linker peptide sequence comprises a sequence that is at least 80% identical to any one of SEQ ID NOs: 71-73 and 124-126.

96. The polypeptide complex of claim 94 or 95, wherein the first polypeptide comprises a sequence that is at least 80% identical to SEQ ID NO: 96 or 97; and/or wherein the second polypeptide comprises a sequence that is at least 80% identical to SEQ ID NO: 98 or 99.

97. A polypeptide complex, comprising
(a) a first polypeptide comprising from N-terminus to C-terminus: a first heavy chain variable region (VH1), a first hinge region, a first Fc region; a first linker peptide sequence, and a first heavy-chain antibody variable domain (VHH1);
(b) a second polypeptide comprising a first light chain variable region (VL1);
(c) a third polypeptide comprising from N-terminus to C-terminus: a second heavy chain variable region (VH2), a second hinge region, a second Fc region, a second linker peptide sequence, and a second heavy-chain antibody variable domain (VHH2); and
(d) a fourth polypeptide comprising a second light chain variable region (VL2);
wherein the VHH1 and/or the VHH2 specifically bind to BCMA or CD38; wherein the VH1 and the VL1 associate with each other and specifically bind to CD3; wherein the VH2 and the VL2 associate with each other and specifically bind to CD3.

98. The polypeptide complex of claim 97, wherein the first linker peptide sequence and/or the second linker peptide sequence comprises a sequence that is at least 80% identical to any one of SEQ ID NOs: 71-73 and 124-126.

99. The polypeptide complex of claim 97 or 98, wherein the first hinge region and/or the second hinge regions comprise a sequence that is at least 80% identical to any one of SEQ ID NOs: 61-65.

100. The polypeptide complex of any one of claims 97-99, wherein sequences of the VHH1 and the VHH2 are identical.

101. The polypeptide complex of claim 100, wherein the first Fc region and/or the second Fc region comprise a sequence that is at least 80% identical to SEQ ID NO: 68.

102. The polypeptide complex of claim 100 or 101, wherein the first polypeptide comprises a sequence that is at least 80% identical to SEQ ID NO: 100 or 101; wherein the second polypeptide comprises a sequence that is at least 80% identical to SEQ ID NO: 102; wherein the third polypeptide comprises a sequence that is at least 80% identical to SEQ ID NO: 100 or 101; and/or wherein the fourth polypeptide comprises a sequence that is at least 80% identical to SEQ ID NO: 102.

103. The polypeptide complex of any one of claims 97-99, wherein sequences of the VHH1 and the VHH2 are different.

104. The polypeptide complex of claim 103, wherein the first Fc region and/or the second Fc region comprise a sequence that is at least 80% identical to SEQ ID NO: 66 or 67.

105. The polypeptide complex of claim 103 or 104, wherein the first polypeptide comprises a sequence that is at least 80% identical to SEQ ID NO: 103 or 104; wherein the second polypeptide comprises a sequence that is at least 80% identical to SEQ ID NO: 107; wherein the third polypeptide comprises a sequence that is at least 80% identical to SEQ ID NO: 105 or 106; and/or wherein the fourth polypeptide comprises a sequence that is at least 80% identical to SEQ ID NO: 107.

106. A polypeptide complex, comprising
(a) a first polypeptide comprising from N-terminus to C-terminus: a first heavy-chain antibody variable domain (VHH1), a first linker peptide sequence, a first heavy chain variable region (VH1), a first hinge region, and a first Fc region;
(b) a second polypeptide comprising a first light chain variable region (VL1);
(c) a third polypeptide comprising from N-terminus to C-terminus: a second heavy-chain antibody variable domain (VHH2), a second linker peptide sequence, a second heavy chain variable region (VH2), a second hinge region, and a second Fc region; and
(d) a fourth polypeptide comprising a second light chain variable region (VL2);
wherein the VHH1 and the VHH2 specifically bind to BCMA or CD38; wherein the VH1 and the VL1 associate with each other and specifically bind to CD3; wherein the VH2 and the VL2 associate with each other and specifically bind to CD3.

107. The polypeptide complex of claim 106, wherein the first linker peptide sequence and/or the second linker peptide sequence comprises a sequence that is at least 80% identical to any one of SEQ ID NOs: 71-73 and 124-126.

108. The polypeptide complex of claim 106 or 107, wherein the first hinge region and/or the second hinge regions comprise a sequence that is at least 80% identical to any one of SEQ ID NOs: 61-65.

109. The polypeptide complex of any one of claims 106-108, wherein sequences of the VHH1 and the VHH2 are identical.

110. The polypeptide complex of claim 109, wherein the first Fc region and/or the second Fc region comprise a sequence that is at least 80% identical to SEQ ID NO: 68.

111. The polypeptide complex of claim 109 or 110, wherein the first polypeptide comprises a sequence that is at least 80% identical to SEQ ID NO: 108 or 109; wherein the second polypeptide comprises a sequence that is at least 80% identical to SEQ ID NO: 110; wherein the third polypeptide comprises a sequence that is at least 80% identical to SEQ ID NO: 108 or 109; and/or wherein the fourth polypeptide comprises a sequence that is at least 80% identical to SEQ ID NO: 110.

112. The polypeptide complex of any one of claims 106-108, wherein sequences of the VHH1 and the VHH2 are different.

113. The polypeptide complex of claim 112, wherein the first Fc region and/or the second Fc region comprise a sequence that is at least 80% identical to SEQ ID NO: 66 or 67.

114. The polypeptide complex of claim 112 or 113, wherein the first polypeptide comprises a sequence that is at least 80% identical to SEQ ID NO: 111 or 112; wherein the second polypeptide comprises a sequence that is at least 80% identical to SEQ ID NO: 115; wherein the third polypeptide comprises a sequence that is at least 80% identical to SEQ ID NO: 113 or 114; and/or wherein the fourth polypeptide comprises a sequence that is at least 80% identical to SEQ ID NO: 115.

115. A nucleic acid comprising a polynucleotide encoding the antibody or antigen-binding fragment thereof of any one of claims 1-56, the multi-specific antibody or antigen-binding fragment thereof of any one of claims 57-74, or the polypeptide complex of any one of claims 75-114.

116. The nucleic acid of claim 115, wherein the nucleic acid is a DNA (e.g., cDNA) or RNA (e.g., mRNA).

117. A vector comprising one or more of the nucleic acids of claim 115 or 116.

118. A cell comprising the vector of claim 117.

119. The cell of claim 118, wherein the cell is a CHO cell.

120. A cell comprising one or more of the nucleic acids of claim 115 or 116.

121. A method of producing an antibody or an antigen-binding fragment thereof, the method comprising
(a) culturing the cell of any one of claims 118-120 under conditions sufficient for the cell to produce the antibody or the antigen-binding fragment; and
(b) collecting the antibody or the antigen-binding fragment produced by the cell.

122. An antibody-drug conjugate comprising the antibody or antigen-binding fragment thereof of any one of claims 1-56, or the multi-specific antibody or antigen-binding fragment thereof of any one of claims 57-74, covalently bound to a therapeutic agent.

123. The antibody drug conjugate of claim 122, wherein the therapeutic agent is a cytotoxic or cytostatic agent.

124. A method of treating a subject having cancer, the method comprising administering a therapeutically effective amount of a composition comprising the antibody or antigen-binding fragment thereof of any one of claims 1-56, the multi-specific antibody or antigen-binding fragment thereof of any one of claims 57-74, the polypeptide complex of any one of claims 75-114, or the antibody-drug conjugate of claim 122 or 123, to the subject.

125. The method of claim 124, wherein the subject has a cancer expressing BCMA or CD38.

126. The method of claim 125, wherein the cancer is multiple myeloma.

127. The method of claim 125, wherein the cancer is hematological malignancy.

128. A method of decreasing the rate of tumor growth, the method comprising contacting a tumor cell with an effective amount of a composition comprising the antibody or antigen-binding fragment thereof of any one of claims 1-56, the multi-specific antibody or antigen-binding fragment thereof of any one of claims 57-74, the polypeptide complex of any one of claims 75-114, or the antibody-drug conjugate of claim 122 or 123.

129. A method of killing a tumor cell, the method comprising contacting a tumor cell with an effective amount of a composition comprising the antibody or antigen-binding fragment thereof of any one of claims 1-56, the multi-specific antibody or antigen-binding fragment thereof of any one of claims 57-74, the polypeptide complex of any one of claims 75-114, or the antibody-drug conjugate of claim 122 or 123.

130. A method of killing a tumor cell, the method comprising contacting a tumor cell with an effective amount of a composition comprising the antibody or antigen-binding fragment thereof of any one of claims 1-56, the multi-specific antibody or antigen-binding fragment thereof of any one of claims 57-74, the polypeptide complex of any one of claims 75-114, or the antibody-drug conjugate of claim 122 or 123.

131. A pharmaceutical composition comprising the antibody or antigen-binding fragment thereof of any one of claims 1-56, the multi-specific antibody or antigen-binding fragment thereof of any one of claims 57-74, or the polypeptide complex of any one of claims 75-114, and a pharmaceutically acceptable carrier.

132. A pharmaceutical composition comprising the antibody-drug conjugate of claim 122 or 123, and a pharmaceutically acceptable carrier.
